(19) United States
(12) Patent Application Publication
(10) Pub. No.: US 2026/0224665 A1
(43) Pub. Date: Aug. 6, 2026

(54) METHODS OF TREATING A TUMOR WITH AN UNMETHYLATED MGMT PROMOTER

(71) Applicants: NEOIMMUNETECH, INC., Rockville, MD (US); WASHINGTON UNIVERSITY, St. Louis, MO (US)

(72) Inventors: Byung Ha LEE, Rockville, MD (US); Sara FERRANDO-MARTINEZ, Washington, DC (US); Jian CAMPIAN, St. Louis, MO (US)

(73) Assignees: NEOIMMUNETECH, INC., Rockville, MD (US); WASHINGTON UNIVERSITY, St. Louis, MO (US)

(21) Appl. No.: 19/127,810

(22) PCT Filed: Nov. 7, 2023
(86) PCT No.: PCT/US2023/078934

§ 371 Date: May 7, 2025

Related U.S. Application Data
(60) Provisional application No. 63/382,689, filed on Nov. 7, 2022.

(51) Int. Cl. A61K 38/20; A61K 31/495; A61P 35/00; C12Q 1/6886

(52) U.S. Cl. CPC A61K 38/2046; A61K 31/495; A61P 35/00; C12Q 1/6886; C12Q 2600/106; C12Q 2600/154

(57) ABSTRACT

The present disclosure relates to methods of treating a tumor (or cancer) with an unmethylated MGMT promoter in a subject in need thereof. In some aspects, the method comprises administering to the subject an anti-cancer treatment, comprising an IL-7 protein alone or in combination with an additional therapeutic agent (e.g., chemotherapy). Some aspects of the present disclosure are directed to methods of identifying cancer subjects (e.g., having a glioblastoma) suitable for the anti-cancer treatment.

### CROSS-REFERENCE TO RELATED APPLICATION

This PCT application claims the priority benefit of U.S. Provisional Application No. 63/382,689, filed on Nov. 7, 2022, which is herein incorporated by reference in its entirety.

### REFERENCE TO SEQUENCE LISTING SUBMITTED ELECTRONICALLY

The content of the sequence listing is submitted electronically (Name: 4241 038PC01_Seqlisting_ST26. xml; Size: 103,771 bytes; and Date of Creation: Nov. 6, 2023) with the application and herein incorporated by reference in its entirety.

### FIELD OF DISCLOSURE

The present disclosure generally relates to the use of an IL-7 protein (e.g., long-acting IL-7 protein) to treat tumors that comprise an unmethylated O⁶-methylguanine-DNA methyltransferase (MGMT) promoter in a subject, e.g., human subjects.

### BACKGROUND OF THE DISCLOSURE

Glioblastoma (GBM) is the most common and aggressive primary brain tumor in adults. Prognosis for GBM remains extremely poor, despite multimodal treatment by surgery, radiotherapy, and chemotherapy. In particular, studies have shown that such treatments have some efficacy in GBM with methylated O⁶-methylguanine DNA methyltransferase (MGMT) promoter. But for patients with GBM that comprise unmethylated MGMT promoter, such treatments (like any other available treatment options available in the art) have minimal efficacy. Accordingly, there remains a need for new treatment options with acceptable safety profile and high efficacy in cancer patients, and in particular, those suffering from a tumor with unmethylated MGMT promoter.

### SUMMARY OF THE DISCLOSURE

Provided herein is a method of treating a tumor in a subject in need thereof, comprising administering to the subject an anti-cancer treatment, wherein a tumor sample obtained from the subject comprises an O⁶-methylguanine-DNA methyltransferase (MGMT) promoter which is unmethylated, and wherein the anti-cancer treatment comprises an interleukin-7 (IL-7) protein. In some aspects, the method comprises prior to the administering, determining a methylation status of the MGMT promoter in the tumor sample obtained from the subject.

Provided herein is a method of identifying a subject suitable for an anti-cancer treatment, comprising determining a methylation status of an unmethylated O⁶-methylguanine-DNA methyltransferase (MGMT) promoter in a tumor sample obtained from the subject, wherein the subject is suitable for the anti-cancer treatment if the MGMT promoter is unmethylated; and wherein the anti-cancer treatment comprises an interleukin-7 (IL-7) protein. In some aspects, the method further comprises administering the anti-cancer treatment to the subject who has been identified as being suitable for the anti-cancer treatment.

Also provided herein is a method of increasing an anti-tumor immune response in a subject in need thereof, comprising administering to the subject an anti-cancer treatment, wherein the subject has a tumor which comprises an O⁶-methylguanine-DNA methyltransferase (MGMT) promoter that is unmethylated, and wherein the anti-cancer treatment comprises an interleukin-7 (IL-7) protein. In some aspects, the method comprises prior to the administering, determining a methylation status of the MGMT promoter in a tumor sample obtained from the subject. In some aspects, after the administering, the anti-tumor immune response in the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold, as compared to a reference subject (e.g., the subject prior to the administering).

For any of the methods provided herein (e.g., methods provided above), in some aspects, after the administering, a median progression-free survival (mPFS) of the subject is increased. In some aspects, the mPFS of the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold, as compared to a reference subject (e.g., the subject prior to the administering). In some aspects, after the administering, the mPFS of the subject is at least about 8 months, at least about 9 months, at least about 10 months, at least about 11 months, or at least about 12 months.

For any of the methods provided herein (e.g., methods provided above), in some aspects, after the administering, a median overall survival (mOS) of the subject is increased. In some aspects, the mOS of the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold, as compared to a reference subject (e.g., the subject prior to the administering). In some aspects, after the administering, the mOS of the subject is at least about 12 months, at least about 13 months, at least about 14 months, at least about 15 months, at least about 16 months, at least about 17 months, at least about 18 months, at least about 19 months, or at least about 20 months.

For any of the methods provided herein (e.g., methods provided above), in some aspects, after the administering, an absolute lymphocyte count (ALC) of the subject is increased. In some aspects, the ALC of the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold, as compared to a reference subject (e.g., the subject prior to the administering). In some aspects, the increase in the ALC persists in the subject for at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, or at least about 15 weeks.

For any of the methods provided herein (e.g., methods provided above), in some aspects, after the administering, the number of tumor-infiltrating lymphocytes (TILs) in the tumor of the subject is increased. In some aspects, the number of TILs in the tumor of the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold, as compared to a reference subject (e.g., the subject prior to the administering).

For any of the methods provided herein, in some aspects, the anti-cancer treatment comprises an additional therapeutic agent. In some aspects, the additional therapeutic agent comprises a standard of care (SOC). In some aspects, the additional therapeutic agent comprises a radiation therapy (RT), chemotherapy, hormone therapy, immunotherapy, photodynamic therapy, stem cell transplant, or combinations thereof. In some aspects, the additional therapeutic agent comprises a chemotherapy. In some aspects, the chemotherapy comprises temozolomide (TMZ). In some aspects, the immunotherapy comprises an immune checkpoint inhibitor, immune checkpoint activator, adoptive cell therapy, or combinations thereof. In some aspects, the immune checkpoint inhibitor comprises a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or a combination thereof. In some aspects, the immune checkpoint activator comprises an OX40 agonist (e.g., anti-OX40 antibody), LAG-3 agonist (e.g. anti-LAG-3 antibody), 4-1BB (CD137) agonist (e.g., anti-CD137 antibody), GITR agonist (e.g., anti-GITR antibody), or a combination thereof.

For any of the methods provided herein, in some aspects, the tumor comprises a glioma. In some aspects, the glioma comprises a high-grade glioma (HGG). In some aspects, the HGG comprises a glioblastoma (GBM), anaplastic astrocytoma, or both. In some aspects, the tumor is newly diagnosed.

For any of the methods provided herein, in some aspects, the methylation status of the MGMT promoter is determined by a methylation specific PCR (MS-PCR), pyrosequencing, high resolution melting, microarray (e.g., Infinium MethylationEPIC), immunohistochemistry (IHC), multiplex ligation-dependent probe amplification (MPLA), or combinations thereof.

In some aspects, the IL-7 protein is not a wild-type IL-7 protein. In some aspects, the IL-7 protein comprises an oligopeptide consisting of 1 to 10 amino acid residues. In some aspects, the oligopeptide comprises methionine (M), glycine (G), methionine-methionine (MM), glycine-glycine (GG), methionine-glycine (MG), glycine-methionine (GM), methionine-methionine-methionine (MMM), methionine-methionine-glycine (MMG), methionine-glycine-methionine (MGM), glycine-methionine-methionine (GMM), methionine-glycine-glycine (MGG), glycine-methionine-glycine (GMG), glycine-glycine-methionine (GGM), glycine-glycine-glycine (GGG), methionine-glycine-glycine-methionine (MGGM) (SEQ ID NO: 41), methionine-methionine-glycine-glycine (MMGG) (SEQ ID NO: 42), glycine-glycine-methionine-methionine (GGMM) (SEQ ID NO: 43), methionine-glycine-methionine-glycine (MGMG) (SEQ ID NO: 44), glycine-methionine-methionine-glycine (GMMG) (SEQ ID NO: 45), glycine-glycine-glycine-methionine (GGGM) (SEQ ID NO: 46), methionine-glycine-glycine-glycine (MGGG) (SEQ ID NO: 47), glycine-methionine-glycine-glycine (GMGG) (SEQ ID NO: 48), glycine-glycine-methionine-glycine (GGMG) (SEQ ID NO: 49), glycine-glycine-methionine-methionine-methionine (GGMMM) (SEQ ID NO: 50), glycine-glycine-glycine-methionine-methionine (GGGMM) (SEQ ID NO: 51), glycine-glycine-glycine-glycine-methionine (GGGGM) (SEQ ID NO: 52), methionine-glycine-methionine-methionine-methionine (MGMMM) (SEQ ID NO: 53), methionine-glycine-glycine-methionine-methionine (MGGMM) (SEQ ID NO: 54), methionine-glycine-glycine-glycine-methionine (MGGGM) (SEQ ID NO: 55), methionine-methionine-glycine-methionine-methionine (MMGMM) (SEQ ID NO: 56), methionine-methionine-glycine-glycine-methionine (MMGGM) (SEQ ID NO: 57), methionine-methionine-glycine-glycine-glycine (MMGGG) (SEQ ID NO: 58), methionine-methionine-methionine-glycine-methionine (MMMGM) (SEQ ID NO: 59), methionine-glycine-methionine-glycine-methionine (MGMGM) (SEQ ID NO: 60), glycine-methionine-glycine-methionine-glycine (GMGMG) (SEQ ID NO: 61), glycine-methionine-methionine-methionine-glycine (GMMMG) (SEQ ID NO: 62), glycine-glycine-methionine-glycine-methionine (GGMGM) (SEQ ID NO: 63), glycine-glycine-methionine-methionine-glycine (GGMMG) (SEQ ID NO: 64), glycine-methionine-methionine-glycine-methionine (GMMGM) (SEQ ID NO: 65), methionine-glycien-methionine-methionine-glycine (MGMMG) (SEQ ID NO: 66), glycine-methionine-glycine-glycine-methionine (GMGGM) (SEQ ID NO: 67), methionine-methionine-glycine-methionine-glycine (MMGMG) (SEQ ID NO: 68), glycine-methionine-methionine-glycine-glycine (GMMGG) (SEQ ID NO: 69), glycine-methionine-glycine-glycine-glycine (GMGGG) (SEQ ID NO: 70), glycine-glycine-methionine-glycine-glycine (GGMGG) (SEQ ID NO: 71), glycine-glycine-glycine-glycine-glycine (GGGGG) (SEQ ID NO: 72), or combinations thereof. In some aspects, the oligopeptide is methionine-glycine-methionine (MGM).

In some aspects, the IL-7 protein comprises a half-life extending moiety. In some aspects, the half-life extending moiety comprises an Fc, albumin, an albumin-binding polypeptide, Pro/Ala/Ser (PAS), a C-terminal peptide (CTP) of the β subunit of human chorionic gonadotropin, polyethylene glycol (PEG), long unstructured hydrophilic sequences of amino acids (XTEN), hydroxyethyl starch (HES), an albumin-binding small molecule, or a combination thereof. In some aspects, the half-life extending moiety is a Fc. In some aspects, the Fc is a hybrid Fc, comprising a hinge region, a CH2 domain, and a CH3 domain, wherein the hinge region comprises a human IgD hinge region, wherein the CH2 domain comprises a part of human IgD CH2 domain and a part of human IgG4 CH2 domain, and wherein the CH3 domain comprises a part of human IgG4 CH3 domain.

In some aspects, the IL-7 protein comprises an amino acid sequence having a sequence identity of at least about 70%, at least about 75%, at least about 80%, at least about 85%, at least about 90%, at least about 95%, at least about 96%, at least about 97%, at least about 98%, at least about 99%, or about 100% to SEQ ID NOs: 1-6, 80-85, and 15-27.

In some aspects, the IL-7 protein is administered at a dose of greater than about 30 μg/kg, greater than about 60 μg/kg, greater than about 90 μg/kg, greater than about 120 μg/kg, greater than about 150 μg/kg, greater than about 180 μg/kg, greater than about 210 μg/kg, greater than about 240 μg/kg, greater than about 270 μg/kg, greater than about 300 μg/kg, greater than about 400 μg/kg, greater than about 500 μg/kg, greater than about 600 μg/kg, greater than about 700 μg/kg, greater than about 800 μg/kg, greater than about 900 μg/kg, greater than about 1,000 μg/kg, greater than about 1,100 μg/kg, greater than about 1,200 μg/kg, greater than about 1,300 μg/kg, greater than about 1,400 μg/kg, greater than about 1,500 μg/kg, greater than about 1,600 μg/kg, greater than about 1,700 μg/kg, greater than about 1,800 μg/kg, greater than about 1,900 μg/kg, or greater than about 2,000 μg/kg. In some aspects, the IL-7 protein is administered at a dose of between about 60 μg/kg and about 1,200 μg/kg, between about 120 μg/kg and about 1,200 μg/kg, between about 240 μg/kg and about 1,200 μg/kg, between about 540 μg/kg and about 1,200 μg/kg, between about 610 μg/kg and about 1,200 μg/kg, between about 650 μg/kg and about 1,200 μg/kg, between about 700 μg/kg and about 1,200 μg/kg, between about 720 μg/kg and about 1,200 μg/kg, between about 750 μg/kg and about 1,200 μg/kg, between about 800 μg/kg and about 1,200 μg/kg, between about 850 μg/kg and about 1,200 μg/kg, between about 900 μg/kg and about 1,200 μg/kg, between about 950 μg/kg and about 1,200 μg/kg, between about 960 μg/kg and between about 1,200 μg/kg, between about 1,000 μg/kg and about 1,200 μg/kg, between about 1,050 μg/kg and about 1,200 μg/kg, between about 1,100 μg/kg and about 1,200 μg/kg, between about 1,200 μg/kg and about 2,000 μg/kg, between about 1,300 μg/kg and about 2,000 μg/kg, between about 1,500 μg/kg and about 2,000 μg/kg, between about 1,700 μg/kg and about 2,000 μg/kg, between about 610 μg/kg and about 1,000 μg/kg, between about 650 μg/kg and about 1,000 μg/kg, between about 700 μg/kg and about 1,000 μg/kg, between about 750 μg/kg and about 1,000 μg/kg, between about 800 μg/kg and about 1,000 μg/kg, between about 850 μg/kg and about 1,000 μg/kg, between about 900 μg/kg and about 1,000 μg/kg, or between about 950 μg/kg and about 1,000 μg/kg. In some aspects, the IL-7 protein is administered at a dose of between about 60 μg/kg and about 120 μg/kg, between about 120 μg/kg and about 240 μg/kg, between about 240 μg/kg and about 540 μg/kg, between about 540 μg/kg and about 720 μg/kg, between about 720 μg/kg and about 960 μg/kg, between about 700 μg/kg and about 900 μg/kg, between about 750 μg/kg and about 950 μg/kg, between about 700 μg/kg and about 850 μg/kg, between about 750 μg/kg and about 850 μg/kg, between about 700 μg/kg and about 800 μg/kg, between about 800 μg/kg and about 900 μg/kg, between about 750 μg/kg and about 850 μg/kg, or between about 850 μg/kg and about 950 μg/kg. In some aspects, the IL-7 protein is administered at a dose of about 30 μg/kg, about 60 μg/kg, about 90 μg/kg, about 120 μg/kg, about 150 μg/kg, about 180 μg/kg, about 210 μg/kg, about 240 μg/kg, about 270 μg/kg, about 300 μg/kg, about 330 μg/kg, about 360 μg/kg, about 390 μg/kg, about 420 μg/kg, about 450 μg/kg, about 480 μg/kg, about 510 μg/kg, about 540 μg/kg, about 570 μg/kg, about 600 μg/kg, about 630 μg/kg, about 650 μg/kg, about 680 μg/kg, about 700 μg/kg, about 720 μg/kg, about 740 μg/kg, about 750 μg/kg, about 760 μg/kg, about 780 μg/kg, about 800 μg/kg, about 820 μg/kg, about 840 μg/kg, about 850 μg/kg, about 860 μg/kg, about 880 μg/kg, about 900 μg/kg, about 920 μg/kg, about 940 μg/kg, about 950 μg/kg, about 960 μg/kg, about 980 μg/kg, about 1,000 μg/kg, about 1,020 μg/kg, about 1,040 μg/kg, about 1,060 μg/kg, about 1,080 μg/kg, about 1,100 μg/kg, about 1,120 μg/kg, about 1,140 μg/kg, about 1,160 μg/kg, about 1,180 μg/kg, about 1,200 μg/kg, about 1,220 μg/kg, about 1,240 μg/kg, about 1,260 μg/kg, about 1,280 μg/kg, about 1,300 μg/kg, about 1,320 μg/kg, about 1,340 μg/kg, about 1,360 μg/kg, about 1,380 μg/kg, about 1,400 μg/kg, about 1,420 μg/kg, about 1,440 μg/kg, about 1,460 μg/kg, about 1,480 μg/kg, about 1,500 μg/kg, about 1,520 μg/kg, about 1,540 μg/kg, about 1,560 μg/kg, about 1,580 μg/kg, about 1,600 μg/kg, about 1,620 μg/kg, about 1,640 μg/kg, about 1,660 μg/kg, about 1,680 μg/kg, about 1,700 μg/kg, about 1,720 μg/kg, about 1,740 μg/kg, about 1,760 μg/kg, about 1,780 μg/kg, about 1,800 μg/kg, about 1,820 μg/kg, about 1,840 μg/kg, about 1,860 μg/kg, about 1,880 μg/kg, about 1,900 μg/kg, about 1,920 μg/kg, about 1,940 μg/kg, about 1,960 μg/kg, about 1,980 μg/kg, or about 2,000 μg/kg. In some aspects, the IL-7 protein is administered at a dose of about 0.01 nmol/kg, about 0.02 nmol/kg, about 0.03 nmol/kg, about 0.04 nmol/kg, about 0.05 nmol/kg, about 0.1 nmol/kg, about 0.2 nmol/kg, about 0.4 nmol/kg, about 0.6 nmol/kg, about 0.8 nmol/kg, about 1 nmol/kg, about 1.2 nmol/kg, about 1.4 nmol/kg, about 1.6 nmol/kg, about 1.8 nmol/kg, about 2 nmol/kg, about 2.2 nmol/kg, about 2.4 nmol/kg, about 2.6 nmol/kg, about 2.8 nmol/kg, about 3 nmol/kg, about 3.5 nmol/kg, about 4 nmol/kg, about 4.5 nmol/kg, about 5 nmol/kg, about 6 nmol/kg, about 7 nmol/kg, about 8 nmol/kg, about 9 nmol/kg, about 10 nmol/kg, about 11 nmol/kg, about 12 nmol/kg, about 13 nmol/kg, about 14 nmol/kg, about 15 nmol/kg, about 16 nmol/kg, about 17 nmol/kg, about 18 nmol/kg, about 19 nmol/kg, about 20 nmol/kg, about 22 nmol/kg, about 24 nmol/kg, about 26 nmol/kg, about 28 nmol/kg, about 30 nmol/kg, about 32 nmol/kg, about 34 nmol/kg, about 36 nmol/kg, about 38 nmol/kg, or about 40 nmol/kg.

In some aspects, the IL-7 protein is administered at a dosing frequency of once about a week, once in about two weeks, once in about three weeks, once in about four weeks, once in about five weeks, once in about six weeks, once in about seven weeks, once in about eight weeks, once in about nine weeks, once in about 10 weeks, once in about 11 weeks, or once in about 12 weeks. In some aspects, the IL-7 protein is administered to the subject at a dose of about 720 μg/kg and a dosing frequency of once in about 12 weeks.

For any of the methods provided herein wherein the anti-cancer treatment comprising an additional agent, in some aspects, the additional agent is administered to the subject at a dosing frequency of once about a week, once in about two weeks, once in about three weeks, once in about four weeks, or once in about five weeks.

In some aspects, the IL-7 protein and the additional agent are administered to the subject concurrently. In some aspects, the IL-7 protein and the additional agent are administered to the subject sequentially. In some aspects, the additional agent is administered to the subject after the IL-7 protein.

In some aspects, the subject receives at least about two doses, at least about three doses, at least about four doses, or at least about five doses of the IL-7 protein. In some aspects, the subject receives at least about two doses, at least about three doses, at least about four doses, at least about five doses, at least about six doses, or at least about seven doses of the additional agent. In some aspects, the subject receives at least about four doses of the IL-7 protein and at least about six doses of the additional agent. In some aspects, the IL-7 protein is administered to the subject once about every 12 weeks and the additional agent is administered to the subject once about every four weeks. In some aspects, one or more doses of the additional agent is administered to the subject over a course of about a single day, over a course of about two consecutive days, over a course of about three consecutive days, over a course of about four consecutive days, over a course of about five consecutive days, over a course of about six consecutive days, or over a course of about seven consecutive days. In some aspects, the additional agent is administered to the subject on days 1-5 of a 28-day cycle.

For any of the methods provided herein, in some aspects, the subject has not previously received an anti-tumor therapy. In some aspects, the subject has previously received an anti-tumor therapy. In some aspects, the anti-tumor therapy comprises a standard of care (SOC). In some aspects, the anti-tumor therapy comprises a radiation therapy (RT), chemotherapy, hormone therapy, immunotherapy, photodynamic therapy, stem cell transplant, or combinations thereof. In some aspects, the anti-tumor therapy comprises both RT and chemotherapy. In some aspects, the chemotherapy comprises temozolomide (TMZ).

In some aspects, the IL-7 protein is administered to the subject intramuscularly, parenthetically, subcutaneously, ophthalmic, intravenously, intraperitoneally, intradermally, intraorbitally, intracerebrally, intracranially, intraspinally, intraventricular, intrathecally, intracistemally, intracapsularly, or intratumorally. In some aspects, the additional agent is administered to the subject intramuscularly, parenthetically, subcutaneously, ophthalmic, intravenously, intraperitoneally, intradermally, intraorbitally, intracerebrally, intracranially, intraspinally, intraventricular, intrathecally, intracistemally, intracapsularly, or intratumorally.

### BRIEF DESCRIPTION OF THE FIGURES

FIG. 1 provides a schematic of the phase I study described in Example 1.

FIGS. 2A and 2B provide comparison of absolute lymphocyte counts (ALC) in cancer patients at four weeks post treatment with one of the following: (a) placebo+TMZ adjuvant alone (i.e., no steroid), (b) steroid administration followed by placebo in combination with TMZ adjuvant, (c) a long-acting IL-7 protein+TMZ adjuvant alone (i.e., no steroid), or (d) steroid administration followed by a long-acting IL-7 protein in combination with TMZ adjuvant. FIG. 2A shows the ALC both at baseline (i.e., prior to treatment) (“BSL”) and at four weeks post treatment (“W4”). FIG. 2B shows the fold-change in ALC between baseline and at four weeks post treatment. In each of the figures, black circles represent patients that did not receive the steroid and gray circles represent patients that received the steroid.

FIGS. 3A and 3B show whether steroid administration has an effect on IL-7 protein-mediated increase in ALC observed in cancer patients. FIG. 3A provide comparison of ALC at baseline (i.e., prior to treatment) (“BSL”) and at four weeks post treatment with one of the following: (a) steroid administration followed by placebo in combination with TMZ adjuvant or (b) steroid administration followed by long-acting IL-7 protein in combination with TMZ adjuvant. FIG. 3B provide comparison of ALC at baseline (i.e., prior to treatment) (“BSL”) and at four weeks post treatment with one of the following: (a) placebo+TMZ adjuvant alone (i.e., no steroid) or (b) long-acting IL-7 protein+TMZ adjuvant alone (i.e., no steroid).

FIGS. 4A and 4B provide comparison of the effect of IL-7 protein administration on ALC in cancer patients with methylated and unmethylated MGMT promoter, respectively. Both patients with methylated MGMT promoter (FIG. 4A) and unmethylated MGMT promoter (FIG. 4B) were treated with either placebo (alone or in combination with TMZ adjuvant) or a long-acting IL-7 protein (alone or in combination with TMZ adjuvant). ALC was measured both at baseline (i.e., prior to treatment) (“BSL”) and at four weeks post treatment (“W4”).

### DETAILED DESCRIPTION OF THE DISCLOSURE

The present disclosure is generally directed to treating certain tumors with an anti-cancer treatment comprising an IL-7 protein. As demonstrated herein, such anti-cancer treatment is particularly useful in treating tumors that comprise an unmethylated O⁶-methylguanine-DNA methyltransferase (MGMT) promoter (e.g., high-grade glioma).

Additional aspects of the present disclosure are provided throughout the present application.

Before the present disclosure is described in greater detail, it is to be understood that this disclosure is not limited to the particular compositions or methods described, which can, of course, vary. As will be apparent to those of skill in the art upon reading this disclosure, each of the individual aspects described and illustrated herein has discrete components and features which can be readily separated from, or combined with, the features of any of the other several aspects without departing from the scope or spirit of the present disclosure. Any recited method can be carried out in the order of events recited or in any other order which is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

The term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language

“comprising,” otherwise analogous aspects described in terms of “consisting of” and/or “consisting essentially of” are also provided. The term “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed subject matter. The present disclosure contemplates aspects of the disclosed compositions and methods corresponding to the scope of each of these phrases. Thus, a composition or method comprising recited elements or steps contemplates particular aspects in which the composition or method consists essentially of or consists of those elements or steps.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary of Biochemistry and Molecular Biology, 2nd ed., 2008, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited (e.g., 10), it is to be understood that values that are about the same quantity or amount as the recited value (e.g., +10%) are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “interleukin-7” or “IL-7” refers to IL-7 polypeptides and derivatives and analogs thereof having substantial amino acid sequence identity to wild-type mature mammalian IL-7s and substantially equivalent biological activity, e.g., in standard bioassays or assays of IL-7 receptor binding affinity. For example, IL-7 refers to an amino acid sequence of a recombinant or non-recombinant polypeptide having an amino acid sequence of: i) a native or naturally-occurring allelic variant of an IL-7 polypeptide, ii) a biologically active fragment of an IL-7 polypeptide, iii) a biologically active polypeptide analog of an IL-7 polypeptide, or iv) a biologically active variant of an IL-7 polypeptide. IL-7 polypeptides of the present disclosure can be obtained from any species, e.g., human, cow or sheep. IL-7 nucleic acid and amino acid sequences are well known in the art. For example, the human IL-7 amino acid sequence has a Genbank accession number of P13232 (SEQ ID NO: 1); the mouse IL-7 amino acid sequence has a Genbank accession number of P10168 (SEQ ID NO: 3); the rat IL-7 amino acid sequence has a Genbank accession number of P56478 (SEQ ID NO: 2); the monkey IL-7 amino acid sequence has a Genbank accession number of NP_001279008 (SEQ ID NO: 4); the cow IL-7 amino acid sequence has a Genbank accession number of P26895 (SEQ ID NO: 5); and the sheep IL-7 amino acid sequence has a Genbank accession number of Q28540 (SEQ ID NO: 6). Protein sequences for exemplary IL-7 proteins are provided in Table 2 (below). In some aspects, an IL-7 polypeptide of the present disclosure is a variant of an IL-7 protein.

As used herein, “administering” refers to the physical introduction of a therapeutic agent or a composition comprising a therapeutic agent to a subject, using any of the various methods and delivery systems known to those skilled in the art. The different routes of administration for a therapeutic agent described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, intratracheal, pulmonary, subcutaneous, subcuticular, intraarticular, subcapsular, subarachnoid, intraventricle, intravitreal, epidural, and intrasternal injection and infusion, as well as in vivo electroporation. Alternatively, a therapeutic agent described herein can be administered via a non-parenteral route, such as a topical, epidermal, or mucosal route of administration, for example, intranasally, orally, vaginally, rectally, sublingually, or topically. Administering can also be performed, for example, once, a plurality of times, and/or over one or more extended periods.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

A “polypeptide” refers to a chain comprising at least two consecutively linked amino acid residues, with no upper limit on the length of the chain. One or more amino acid residues in the protein can contain a modification such as, but not limited to, glycosylation, phosphorylation or disulfide bond formation. A “protein” can comprise one or more polypeptides. Unless otherwise specified, the terms “protein” and “polypeptide” can be used interchangeably.

The term “nucleic acid molecule,” as used herein, is intended to include DNA molecules and RNA molecules. A nucleic acid molecule can be single-stranded or double-stranded, and can be cDNA.

“Conservative amino acid substitutions” refer to substitutions of an amino acid residue with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In some aspects, a predicted nonessential amino acid residue in an antibody is replaced with another amino acid residue from the same side chain family. Methods of identifying nucleotide and amino acid conservative substitutions which do not eliminate antigen binding are well-known in the art (see, e.g., Brummell et al., Biochem. 32:1180-1187 (1993); Kobayashi et al. Protein Eng. 12 (10): 879-884 (1999); and Burks et al. Proc. Natl. Acad. Sci. USA 94:412-417 (1997)).

For nucleic acids, the term “substantial homology” indicates that two nucleic acids, or designated sequences thereof, when optimally aligned and compared, are identical, with appropriate nucleotide insertions or deletions, in at least about 80% of the nucleotides, at least about 90% to 95%, or at least about 98% to 99.5% of the nucleotides. Alternatively, substantial homology exists when the segments will hybridize under selective hybridization conditions, to the complement of the strand.

For polypeptides, the term “substantial homology” indicates that two polypeptides, or designated sequences thereof, when optimally aligned and compared, are identical, with appropriate amino acid insertions or deletions, in at least about 80% of the amino acids, at least about 90% to 95%, or at least about 98% to 99.5% of the amino acids.

The percent identity between two sequences is a function of the number of identical positions shared by the sequences (i.e., % homology= # of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, e.g., as described in the non-limiting examples below.

The percent identity between two nucleotide sequences can be determined using the GAP program in the GCG software package (available at worldwideweb.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48): 444-453 (1970)) algorithm which has been incorporated into the GAP program in the GCG software package (available at worldwideweb.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules described herein. BLAST protein searches can be performed with the XBLAST program, score=50, wordlength=3 to obtain amino acid sequences homologous to the protein molecules described herein. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., XBLAST and NBLAST) can be used. See worldwideweb.ncbi.nlm.nih.gov.

The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., the other parts of the chromosome) or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

Nucleic acids, e.g., cDNA, can be mutated, in accordance with standard techniques to provide gene sequences. For coding sequences, these mutations, can affect amino acid sequence as desired. In particular, DNA sequences substantially homologous to or derived from native V, D, J, constant, switches and other such sequences described herein are contemplated (where “derived” indicates that a sequence is identical or modified from another sequence).

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Some vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, some vectors are capable of directing the expression of genes to which they are operatively linked. Such vectors are referred to herein as “recombinant expression vectors” (or simply, “expression vectors”) In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell that comprises a nucleic acid that is not naturally present in the cell, and can be a cell into which a recombinant expression vector has been introduced. It should be understood that such terms are intended to refer not only to the particular subject cell but to the progeny of such a cell. Because some modifications can occur in succeeding generations due to either mutation or environmental influences, such progeny cannot, in fact, be identical to the parent cell, but are still included within the scope of the term “host cell” as used herein.

As used herein, the term “linked” or “conjugated” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

A “cancer” refers a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. “Cancer” as used herein refers to primary, metastatic and recurrent cancers. As is apparent from the present disclosure, a cancer that can be treated with the present disclosure comprises a tumor, which has an O⁶-methylguanine-DNA methyltransferase (MGMT) promoter which is unmethylated. Non-limiting examples of suitable cancers that can be treated with the present disclosure are provided elsewhere in the present disclosure.

The term “fusion protein” refers to proteins created through the joining of two or more genes that originally coded for separate proteins. Translation of this fusion gene results in a single polypeptide or multiple polypeptides with functional properties derived from each of the original proteins. In some aspects, the two or more genes can comprise a substitution, a deletion, and/or an addition in its nucleotide sequence.

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) and one inhibitory (FcγRIIB) receptor. Various properties of human FcγRs are known in the art. The majority of innate effector cell types coexpress one or more activating FcγR and the inhibitory FcγRIIB, whereas natural killer (NK) cells selectively express one activating Fc receptor (FcγRIII in mice and FcγRIIIA in humans) but not the inhibitory FcγRIIB in mice and humans. Human IgG1 binds to most human Fc receptors and is considered equivalent to murine IgG2a with respect to the types of activating Fc receptors that it binds to.

An “Fc region” (fragment crystallizable region) or “Fc domain” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (C1q) of the classical complement system. Thus, an Fc region comprises the constant region of an antibody excluding the first constant region immunoglobulin domain (e.g., CH1 or CL). In IgG, IgA and IgD antibody isotypes, the Fc region comprises two identical protein fragments, derived from the second (CH2) and third (CH3) constant domains of the antibody's two heavy chains; IgM and IgE Fc regions comprise three heavy chain constant domains (CH domains 2-4) in each polypeptide chain. For IgG, the Fc region comprises immunoglobulin domains CH2 and CH3 and the hinge between CH1 and CH2 domains. Although the definition of the boundaries of the Fc region of an immunoglobulin heavy chain might vary, as defined herein, the human IgG heavy chain Fc region is defined to stretch from an amino acid residue D221 for IgG1, V222 for IgG2, L221 for IgG3 and P224 for IgG4 to the carboxy-terminus of the heavy chain, wherein the numbering is according to the EU index as in Kabat. The CH2 domain of a human IgG Fc region extends from amino acid 237 to amino acid 340, and the CH3 domain is positioned on C-terminal side of a CH2 domain in an Fc region, i.e., it extends from amino acid 341 to amino acid 447 or 446 (if the C-terminal lysine residue is absent) or 445 (if the C-terminal glycine and lysine residues are absent) of an IgG. As used herein, the Fc region can be a native sequence Fc, including any allotypic variant, or a variant Fc (e.g., a non-naturally occurring Fc). Fc can also refer to this region in isolation or in the context of an Fc-comprising protein polypeptide such as a “binding protein comprising an Fc region,” also referred to as an “Fc fusion protein” (e.g., an antibody or immunoadhesion).

A “native sequence Fc region” or “native sequence Fc” comprises an amino acid sequence that is identical to the amino acid sequence of an Fc region found in nature. Native sequence human Fc regions include a native sequence human IgG1 Fc region; native sequence human IgG2 Fc region; native sequence human IgG3 Fc region; and native sequence human IgG4 Fc region as well as naturally occurring variants thereof. Native sequence Fc include the various allotypes of Fcs (see, e.g., Jefferis et al. (2009) mAbs 1:1).

Additionally, a Fc (native or variant) of the present disclosure can be in the form of having native sugar chains, increased sugar chains, or decreased sugar chains compared to the native form, or can be in a deglycosylated form. The immunoglobulin Fc sugar chains can be modified by conventional methods such as a chemical method, an enzymatic method, and a genetic engineering method using a microorganism. The removal of sugar chains from an Fc fragment results in a sharp decrease in binding affinity to the C1q part of the first complement component C1, and a decrease or loss of ADCC or CDC, thereby not inducing any unnecessary immune responses in vivo. In this regard, an immunoglobulin Fc region in a deglycosylated or aglycosylated form can be more suitable to the object of the present disclosure as a drug carrier. As used herein, the term “deglycosylation” refers to an Fc region in which sugars are removed enzymatically from an Fc fragment. Additionally, the term “aglycosylation” means that an Fc fragment is produced in an unglycosylated form by a prokaryote, and preferably in E. coli.

As used herein, the term “immune response” refers to a biological response within a vertebrate against foreign agents, which response protects the organism against these agents and diseases caused by them. For instance, the term “anti-tumor immune response” refers to an immune response against a tumor antigen. An immune response is mediated by the action of a cell of the immune system (e.g., a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4+ or CD8+ T cell, or the inhibition of a Treg cell.

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

The term “effector T cells” (Teff) refers to T cells (e.g., CD4+ and CD8+ T cells) with cytolytic activities as well as T helper (Th) cells, which secrete cytokines and activate and direct other immune cells, but does not include regulatory T cells (Treg cells).

As used herein, the term “regulatory T cells” (Tregs) refer to a population of T cells with the ability to reduce or suppress the induction and proliferation of effector T cells, and thereby, modulate an immune response. In some aspects, Tregs can suppress an immune response by secreting anti-inflammatory cytokines, such as IL-10, TGF-β, and IL-35, which can interfere with the activation and differentiation of naïve T cells into effector T cells. In some aspects, Tregs can also produce cytolytic molecules, such as Granzyme B, which can induce the apoptosis of effector T cells. In some aspects, the regulatory T cells are natural regulatory T cells (nTregs) (i.e., developed within the thymus). In some aspects, the regulatory T cells are induced regulatory T cells (iTregs) (i.e., naïve T cells that differentiate into Tregs in the peripheral tissue upon exposure to certain stimuli). Methods for identifying Tregs are known in the art. For example, Tregs express certain phenotypic markers (e.g., CD25, Foxp3, or CD39) that can be measured using flow cytometry. See, e.g., International Publication No. WO 2017/062035 A1; Gu J., et al., Cell Mol Immunol 14 (6): 521-528 (2017). In some aspects, the Tregs are CD45RA CD39+ T cells.

As used herein, the term “tumor infiltrating lymphocytes” or “TILs” refers to lymphocytes (e.g., effector T cells) that have migrated from the periphery (e.g., from the blood) into a tumor. In some aspects, the tumor infiltrating lymphocytes are CD4+TILs. In some aspects, the tumor infiltrating lymphocytes are CD8+TILs.

An increased ability to stimulate an immune response or the immune system, can result from an enhanced agonist activity of T cell costimulatory receptors and/or an enhanced antagonist activity of inhibitory receptors. An increased ability to stimulate an immune response or the immune system can be reflected by a fold increase of the EC50 or maximal level of activity in an assay that measures an immune response, e.g., an assay that measures changes in cytokine or chemokine release, cytolytic activity (determined directly on target cells or indirectly via detecting CD107a or granzymes) and proliferation. The ability to stimulate an immune response or the immune system activity can be enhanced by at least 10%, 30%, 50%, 75%, 2 fold, 3 fold, 5 fold or more.

A “variant” of an, e.g., IL-7 protein, is defined as an amino acid sequence that is altered by one or more amino acids. The variant can have “conservative” changes, wherein a substituted amino acid has similar structural or chemical properties, e.g., replacement of leucine with isoleucine. More rarely, a variant can have “nonconservative” changes, e.g., replacement of a glycine with a tryptophan. Similar minor variations can also include amino acid deletions or insertions, or both. Guidance in determining which and how many amino acid residues can be substituted, inserted or deleted without abolishing biological activity can be found using computer programs well known in the art, for example software for molecular modeling or for producing alignments. For example, the variant IL-7 proteins included within the disclosure include IL-7 proteins that retain IL-7 activity. Variants which also include additions, substitutions or deletions are also included within the present disclosure as long as the variants retain substantially equivalent biological activity of the non-variant (e.g., wild-type) counterpart. For example, truncations of IL-7 which retain comparable biological activity as the full length form of the IL-7 protein are included within the present disclosure. The activity of a therapeutic agent described herein (e.g., an IL-7 protein and/or an addition therapeutic agent) can be measured using any suitable methods known in the art.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino acid or nucleic acid sequence). The percent identity between the two sequences is a function of the number of identical positions shared by the sequences (i.e., % homology= # of identical positions/total # of positions.times. 100). The determination of percent homology between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs of Altschul, et al., (1990) J. Mol. Biol. 215:403-10. BLAST nucleotide searches can be performed with the NBLAST program, score=100, wordlength=12. BLAST protein searches can be performed with the XBLAST program, score=50, wordlength=3. To obtain gapped alignments for comparison purposes, Gapped BLAST can be utilized as described in Altschul et al., (1997) Nucleic Acids Research 25 (17): 3389-3402. When utilizing BLAST and Gapped BLAST programs, the default parameters of the respective programs (e.g., XBLAST and NBLAST) can be used.

A “subject” includes any human or nonhuman animal. The term “nonhuman animal” includes, but is not limited to, vertebrates such as nonhuman primates, sheep, dogs, and rodents such as mice, rats and guinea pigs. In some aspects, the subject is a human. The terms “subject” and “patient” are used interchangeably herein. Unless indicated otherwise, a subject described herein suffers from one or more of the indications described herein (e.g., tumor).

The term “therapeutically effective amount” or “therapeutically effective dosage” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to prevent or delay other unwanted cell proliferation. In some aspects, an effective amount is an amount sufficient to delay tumor development. In some aspects, an effective amount is an amount sufficient to prevent or delay tumor recurrence. An effective amount can be administered in one or more administrations. The effective amount of the drug or composition can: (i) reduce the number of cancer cells; (ii) reduce tumor size; (iii) inhibit, retard, slow to some extent and can stop cancer cell infiltration into peripheral organs; (iv) inhibit (i.e., slow to some extent and can stop tumor metastasis; (v) inhibit tumor growth; (vi) prevent or delay occurrence and/or recurrence of tumor; and/or (vii) relieve to some extent one or more of the symptoms associated with the cancer. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The term “dosing frequency” or “dosing interval” refers to the number of times a therapeutic agent (e.g., an IL-7 protein and/or an additional therapeutic agent) is administered to a subject within a specific time period. Dosing frequency can be indicated as the number of doses per a given time, for example, once per day, once a week, or once in two weeks. As used herein, “dosing frequency” is applicable where a subject receives multiple (or repeated) administrations of a therapeutic agent. Suitable dosing frequency for any of the IL-7 proteins and additional therapeutic agents described herein are provided elsewhere in the present disclosure.

As used herein, the term “standard of care” refers to a treatment that is accepted by medical experts as a proper treatment for a certain type of disease and that is widely used by healthcare professionals. The term can be used interchangeable with any of the following terms: “best practice,” “standard medical care,” and “standard therapy.”

By way of example, an “anti-cancer agent” promotes cancer regression in a subject or prevents further tumor growth. In some aspects, a therapeutically effective amount of the drug promotes cancer regression to the point of eliminating the cancer. “Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example for the treatment of tumors, a therapeutically effective amount of an anti-cancer agent can inhibit cell growth or tumor growth by at least about 10%, at least about 20%, by at least about 40%, by at least about 60%, or by at least about 80% relative to untreated subjects or, in some aspects, relative to patients treated with a standard-of-care therapy. In some aspects, tumor regression can be observed and continue for a period of at least about 20 days, at least about 40 days, or at least about 60 days. Notwithstanding these ultimate measurements of therapeutic effectiveness, evaluation of immunotherapeutic drugs must also make allowance for “immune-related” response patterns.

As used herein, the term “immune checkpoint inhibitor” refers to molecules that totally or partially reduce, inhibit, interfere with or modulate one or more checkpoint proteins. Checkpoint proteins regulate T-cell activation or function. Numerous checkpoint proteins are known, such as CTLA-4 and its ligands CD80 and CD86; and PD-1 with its ligands PD-L1 and PD-L2. Pardoll, D.M., Nat Rev Cancer 12 (4): 252-64 (2012). These proteins are responsible for co-stimulatory or inhibitory interactions of T-cell responses. Immune checkpoint proteins regulate and maintain self-tolerance and the duration and amplitude of physiological immune responses. Immune checkpoint inhibitors include antibodies or are derived from antibodies. Suitable immune checkpoint inhibitors that are useful for the present disclosure are provided elsewhere in the present disclosure.

As used herein, the term “reference subject” refers to a corresponding subject (e.g., a cancer subject) who did not receive an anti-cancer treatment described herein (e.g., an IL-7 protein alone or in combination with an additional therapeutic agent). The term “reference subject” can also refer to a same subject (i.e., treated with the methods provided herein) but prior to the administration of a combination of an anti-cancer treatment described herein. In some aspects, the term “reference subject” refers to an average of a population of subjects (e.g., cancer subjects).

As used herein, the terms “μg” and “μM” are used interchangeably with “μg” and “μM,” respectively.

Various aspects described herein are described in further detail in the following subsections.

### II. Methods of the Disclosure

Present disclosure is generally directed to methods of treating a tumor (or a cancer) with an anti-cancer treatment comprising an interleukin (IL-7) protein. As demonstrated herein, such anti-cancer treatment is particularly useful in treating tumors that comprise an unmethylated O⁶-methylguanine-DNA methyltransferase (MGMT) promoter. Accordingly, some aspects of the present disclosures are related to methods of treating a tumor with an unmethylated MGMT promoter in a subject in need thereof, comprising administering to the subject an anti-cancer treatment, wherein the anti-cancer treatment comprises an interleukin-7 (IL-7) protein. In some aspects, a treatment method provided herein comprises confirming the methylation status of the MGMT promoter (i.e., determining whether the MGMT promoter is methylated or unmethylated) of the tumor prior to administering an anti-cancer treatment to the subject. For example, in some aspects, provided herein is a method of treating a tumor in a subject in need thereof, comprising determining a methylation status of the MGMT promoter in a tumor sample obtained from the subject, wherein if the MGMT promoter is unmethylated, an anti-cancer treatment comprising an IL-7 protein is administered to the subject.

As is apparent from the present disclosure, in some aspects, by determining the methylation status of the MGMT promoter, it can be possible to assess whether a subject suffering a tumor (also referred to herein as a “cancer subject”) would respond to an anti-cancer treatment provided herein. Accordingly, some aspects of the present disclosure relates to a method of identifying a cancer subject suitable for an anti-cancer treatment comprising an IL-7 protein, wherein the method comprises determining a methylation status of the MGMT promoter in a tumor sample obtained from the subject, and wherein the subject is suitable for the anti-cancer treatment if the MGMT promoter is unmethylated. In some aspects, such a method further comprises administering the anti-cancer treatment to the subject who has been identified as being suitable for the anti-cancer treatment.

Where a method provided herein comprises determining a methylation status of a MGMT promoter, any suitable method known in the art can be used to determine the methylation status. Non-limiting examples of such methods include: a methylation specific PCR (MS-PCR), pyrosequencing, high resolution melting, microarray (e.g., Infinium MethylationEPIC), immunohistochemistry (IHC), multiplex ligation-dependent probe amplification (MPLA), or combinations thereof. Additional disclosure relating to useful methods for determining MGMT methylation status are provided in Brandner et al., Neuro Oncol 23 (9): 1457-1469 (September 2021); and U.S. Pat. No. 10,053,724; each of which is incorporated herein by reference in its entirety.

As demonstrated and described herein, compared to treatments known in the art, anti-cancer treatments provided herein (i.e., comprising an IL-7 protein) exert improved therapeutic effect. Therefore, some aspects of the present disclosure are related to methods of increasing an anti-tumor immune response in a subject in need thereof, comprising administering to the subject an anti-cancer treatment, which comprises an IL-7 protein, and wherein the subject has a tumor which comprises an unmethylated MGMT promoter. In some aspects, such methods comprise determining the methylation status of the MGMT promoter in a tumor sample obtained from the subject prior to administering the anti-cancer treatment.

In some aspects, after the administration, the anti-tumor immune response in the subject is increased by at least about 10%, at least about 20%, at least about 30%, at least about 40%, at least about 50%, at least about 60%, at least about 70%, at least about 80%, at least about 90% or at least about 100%, as compared to a corresponding value in a reference subject (e.g., the subject prior to the administration and/or a corresponding subject that did not receive the administration of the anti-cancer treatment). In some aspects, after the administration, the anti-tumor immune response in the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, as compared to the corresponding value in the reference subject.

In some aspects, an increased anti-tumor immune response can be associated with one or more of the following therapeutic effects: (i) reduced tumor volume and/or growth; (ii) increased effector activity (e.g., ability to kill a tumor cell) of a tumor-specific T cell; (iii) increased number of tumor-specific effector T cells, e.g., in a tumor; (iv) increased number of tumor-infiltrating lymphocytes (TILs), e.g., in a tumor; or (v) any combination thereof. Unless indicated otherwise, the term “number” refers to both absolute number as well as percentage. Any of the therapeutic effects described above or elsewhere in the present disclosure can be determined using any of the suitable methods provided herein and/or known in the art.

In some aspects, after the administration of an anti-cancer treatment provided herein (i.e., comprising an IL-7 protein) to a subject suffering from a tumor with an unmethylated MGMT promoter, a tumor volume in the subject is reduced as compared to a corresponding value in a reference subject (e.g., the subject prior to the administration and/or a corresponding subject that was not treated with the anti-cancer treatment). In some aspects, after the administration, the tumor volume in the subject is reduced by at least about 5%, at least about 10%, at least about 20%, at least about 30%, at least about 40%, at least about 50%, at least about 60%, at least about 70%, at least about 80%, at least about 90%, or at least about 100% as compared to a corresponding value in the reference subject. In some aspects, after the administration of the anti-cancer treatment, a tumor growth in the subject is reduced as compared to a corresponding value in the reference subject. In some aspects, after the administration, the tumor growth in the subject is reduced by at least about 5%, at least about 10%, at least about 20%, at least about 30%, at least about 40%, at least about 50%, at least about 60%, at least about 70%, at least about 80%, at least about 90%, or at least about 100% as compared to the corresponding value in the reference subject.

In some aspects, after the administration of the anti-cancer treatment (i.e., comprising an IL-7 protein), an effector activity of a tumor-specific T cell (e.g., CD8+ T cell and/or CD4+ T cell) in the subject is increased as compared to corresponding value in the reference subject. In some aspects, the effector activity in the subject is increased by at least about 10%, at least about 20%, at least about 30%, at least about 40%, at least about 50%, at least about 60%, at least about 70%, at least about 80%, at least about 90%, or at least about 100%, as compared to a corresponding value in the reference subject. In some aspects, the effector activity in the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold as compared to a corresponding value in the reference subject. In some aspects, the effector activity comprises any cytolytic activity of a T cell (e.g., CD8+ T cell and/or CD4+ T cell) that can be useful in the treatment of a cancer. Non-limiting examples of such effector activity include the ability to produce effector molecules (e.g., cytokines, perforin, and/or granzyme) upon antigen stimulation; ability to recognize and kill tumor cells; or both.

In some aspects, after the administration of an anti-cancer treatment provided herein (e.g., comprising an IL-7 protein) to a subject suffering from a tumor with an unmethylated MGMT promoter, the number of tumor-specific effector T cells (e.g., CD8+ and/or CD4+) in the subject is increased. In some aspects, the number of tumor-specific effector T cells in the subject is increased by at least about 10%, at least about 20%, at least about 30%, at least about 40%, at least about 50%, at least about 60%, at least about 70%, at least about 80%, at least about 90% or at least about 100%, as compared to a corresponding value in the reference subject. In some aspects, the number of tumor-specific effector T cells in the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold as compared to a corresponding value in the reference subject.

Not to be bound by any one theory, in some aspects, the increased number is due to increased proliferation of the tumor-specific effector T cells. Accordingly, in some aspects, after the administration of an anti-cancer treatment provided herein to a subject suffering from a tumor with an unmethylated MGMT promoter, the proliferation of the tumor-specific effector T cells is increased by at least about 10%, at least about 20%, at least about 30%, at least about 40%, at least about 50%, at least about 60%, at least about 70%, at least about 80%, at least about 90% or at least about 100%, as compared to a corresponding value in the reference subject. In some aspects, after the administration of the anti-cancer treatment, the proliferation of the tumor-specific effector T cells is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold compared to a corresponding value in the reference subject. In some aspects, the increase in the number of tumor-specific effector T cells is due to the ability of the T cells to survive and/or persist. In some aspects, after the administration of an anti-cancer treatment (i.e., comprising an IL-7 protein) to a subject suffering from a tumor with an unmethylated MGMT promoter, the ability of the tumor-specific effector T cells to survive and/or persist in the subject is increased. In some aspects, the ability of the tumor-specific effector T cells to survive and/or persist in the subject is increased by at least about 10%, at least about 20%, at least about 30%, at least about 40%, at least about 50%, at least about 60%, at least about 70%, at least about 80%, at least about 90% or at least about 100%, as compared to a corresponding value in the reference subject. In some aspects, the ability of the tumor-specific effector T cells to survive and/or persist in the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold as compared to a corresponding value in the reference subject. In some aspects, after the administration of the anti-cancer treatment, both the proliferation and survival/persistence of the tumor-specific effector T cells is increased in the subject.

In some aspects, the tumor-specific effector T cells are tumor-infiltrating lymphocytes (TILs). Accordingly, in some aspects, after the administration of an anti-cancer treatment described herein (i.e., comprising an IL-7 protein) to a subject suffering from a tumor with an unmethylated MGMT promoter, the number of TILs in a tumor of a subject is increased as compared to a corresponding value in the reference subject. In some aspects, after the administration of the anti-cancer treatment, the number of TILs in a tumor of the subject is increased by at least about 10%, at least about 20%, at least about 30%, at least about 40%, at least about 50%, at least about 60%, at least about 70%, at least about 80%, at least about 90% or at least about 100%, as compared to a corresponding value in the reference subject. In some aspects, after the administration of the anti-cancer treatment, the number of TILs in a tumor of the subject is increase by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold as compared to a corresponding value in the reference subject.

In some aspects, the ability of an anti-cancer treatment provided herein (i.e., comprising an IL-7 protein) to increase the proliferation, survival, and/or persistence of tumor-specific T cells can also increase the overall lymphocyte count (i.e., “absolute lymphocyte count”) in the subject. Accordingly, in some aspects, after the administration of an anti-cancer treatment provided herein (i.e., comprising an IL-7 protein) to a subject suffering from a tumor with an unmethylated MGMT promoter, the absolute lymphocyte count in the subject is increased as compared to a corresponding value in the reference subject. In some aspects, after the administration, the absolute lymphocyte count in the subject is increased by at least about 10%, at least about 20%, at least about 30%, at least about 40%, at least about 50%, at least about 60%, at least about 70%, at least about 80%, at least about 90% or at least about 100%, as compared to a corresponding value in the reference subject. In some aspects, after the administration, the absolute lymphocyte count in the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold as compared to a corresponding value in the reference subject.

In some aspects, after the administration of the anti-cancer treatment, the increase in the absolute lymphocyte count in the subject is maintained much longer as compared to a corresponding value in the reference subject. In some aspects, compared to the reference subject, the increase in the absolute lymphocyte count is maintained for at least about 10%, at least about 20%, at least about 30%, at least about 40%, at least about 50%, at least about 60%, at least about 70%, at least about 80%, at least about 90%, or at least about 100% longer. In some aspects, compared to the reference subject, in the increase in the absolute lymphocyte count is maintained for at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold longer. In some aspects, after the administration of the anti-cancer treatment, the increase in the absolute lymphocyte count is maintained in the subject for at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, or at least about 15 weeks.

As will be apparent to those skilled in the arts, in some aspects, one or more of the therapeutic effects described above can lead to increased overall survival. In some aspects, the increased overall survival comprises progression-free survival. Accordingly, in some aspects, after the administration of an anti-cancer treatment provided herein (i.e., comprising an IL-7 protein) to a subject suffering from a tumor with an unmethylated MGMT promoter, increases the duration of overall survival of the subject. In some aspects, the duration of overall survival of the subject is increased by at least about 10%, at least about 20%, at least about 30%, at least about 40%, at least about 50%, at least about 60%, at least about 70%, at least about 80%, at least about 90% or at least about 100%, as compared to a corresponding value in the reference subject. In some aspects, after the administration, the duration of overall survival of the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, as compared to a corresponding value in the reference subject. In some aspects, after the administration of the anti-cancer treatment, the duration of overall survival of the subject is increased by at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, or at least 24 months. In some aspects, after the administration, the duration of overall survival of the subject is at least about 12 months, at least about 13 months, at least about 14 months, at least about 15 months, at least about 16 months, at last about 17 months, at least about 18 months, at least about 19 months, or at least about 20 months.

In some aspects, after the administration of an anti-cancer treatment described herein (i.e., comprising an IL-7 protein) to a subject suffering from a tumor with an unmethylated MGMT promoter, the duration of progression-free survival of the subject is increased. For example, in some aspects, the progression-free survival of the subject is increased by at least about 10%, at least about 20%, at least about 30%, at least about 40%, at least about 50%, at least about 60%, at least about 70%, at least about 80%, at least about 90% or at least about 100%, as compared to a corresponding value in the reference subject. In some aspects, after the administration, the progression-free survival of the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, as compared to a corresponding value of the reference subject. In some aspects, after the administration, the progression-free survival of the subject is increased by at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, or at least 24 months. In some aspects, after the administration of the anti-cancer treatment, the progression-free survival of the subject is at least about 8 months, at least about 9 months, at least about 10 months, at least about 11 months, or at least about 12 months.

### 1. Combination Treatments:

As described herein, an anti-cancer treatment useful for the present disclosure comprises an IL-7 protein. In some aspects, the anti-cancer treatment comprises one or more additional therapeutic agents. Accordingly, in some aspects, administering an anti-cancer treatment to a subject (e.g., suffering from a tumor with an unmethylated MGMT tumor) comprises administering to the subject an IL-7 protein in combination with an additional therapeutic agent. Unless indicated otherwise, the additional therapeutic agent can comprise any agent known in the art that can be useful in treating a tumor described herein.

In some aspects, the additional therapeutic agent comprises a standard of care (SOC). Accordingly, in some aspects, the methods provided herein comprises administering to a subject (e.g., suffering from a tumor with an unmethylated MGMT promoter) an anti-cancer treatment, wherein the anti-cancer treatment comprises an IL-7 protein and SOC. In some aspects, SOC comprises a surgery, chemotherapy, radiotherapy, or combinations thereof. For example, with glioma, the first stage of SOC generally comprises surgery during which as much of the tumor is removed from the subject. After surgery, the SOC generally comprises radiotherapy, chemotherapy, or both. Accordingly, in some aspects, an anti-cancer treatment useful for the present disclosure comprises an IL-7 protein and SOC, wherein the SOC comprises a surgery. In some aspects, an anti-cancer treatment comprises an IL-7 protein and SOC, wherein the SOC comprises a radiotherapy. In some aspects, an anti-cancer treatment comprises an IL-7 protein and SOC, wherein the SOC comprises a chemotherapy. In some aspects, an anti-cancer treatment comprises an IL-7 protein and SOC, wherein the SOC comprises surgery and radiotherapy. In some aspects, an anti-cancer treatment comprises an IL-7 protein and SOC, wherein the SOC comprises surgery and chemotherapy. In some aspects, an anti-cancer treatment comprises an IL-7 protein and SOC, wherein the SOC comprises radiotherapy and chemotherapy. In some aspects, an anti-cancer treatment comprises an IL-7 protein and SOC, wherein the SOC comprises surgery, radiotherapy, and chemotherapy. For any anti-cancer treatments described herein comprising a chemotherapy, in some aspects, the chemotherapy comprises temozolomide (TMZ). Tan et al., CA Cancer J Clin 70 (4): 299-312 (July 2020). Accordingly, in some aspects, a method provided herein comprises administering to a subject (e.g., suffering from a tumor with an unmethylated MGMT promoter) an anti-cancer treatment, wherein the anti-cancer treatment comprises an IL-7 protein and SOC, wherein the SOC comprises concurrent radiotherapy with TMZ. In some aspects, the anti-cancer treatment comprises an IL-7 protein and SOC, wherein the SOC comprises concurrent radiotherapy with TMZ and further adjuvant TMZ. In some aspects, the further adjuvant TMZ is administered to the subject after the concurrent radiotherapy with TMZ has stopped.

In some aspects, an additional therapeutic agent that can be used in combination with an IL-7 protein described herein comprises additional radiation therapy, additional chemotherapy, hormone therapy, immunotherapy, photodynamic therapy, stem cell transplant, or combinations thereof. Accordingly, in some aspects, an anti-cancer treatment useful for the present disclosure comprises an IL-7 protein and radiation therapy. In some aspects, an anti-cancer treatment useful for the present disclosure comprises an IL-7 protein and a chemotherapy (e.g., TMZ). In some aspects, an anti-cancer treatment comprises an IL-7 protein and a hormone therapy. In some aspects, an anti-cancer treatment comprises an IL-7 protein and a photodynamic therapy. In some aspects, an anti-cancer treatment comprises an IL-7 protein and a stem cell transplant. In some aspects, an anti-cancer treatment comprises an IL-7 protein and an immunotherapy. Non-limiting examples of immunotherapies that can be used include: an immune checkpoint inhibitor, immune checkpoint activator, adoptive cell therapy, or combinations thereof. For example, in some aspects, an anti-cancer treatment useful for the present disclosure comprises an IL-7 protein and an immune checkpoint inhibitor, wherein the immune checkpoint inhibitor comprises a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or a combination thereof. Non-limiting examples of such immune checkpoint inhibitors are known in the art. For example, suitable anti-PD-1 antagonists are described, e.g., in U.S. Pat. Nos. 6,808,710; 7,488,802; 8,008,449; 8,168,757; 8,354,509; 8,609,089; 8,779,105; and 9,217,034; and 7,635,757; 7,943,743; 8,217,149; 8,981,063; and 9,624,298, each of which is incorporated herein by reference in its entirety. Suitable anti-CTLA-4 antagonists are described, e.g., in U.S. Pat. Nos. 5,977,318; 6,051,227; 6,682,736; 6,984,720; 7,034,121; 7,605,238; 10,479,833; and International Publ. No. WO 2007/113648, each of which is incorporated herein by reference in its entirety. Suitable TIM-3 antagonists are described, e.g., in U.S. Pat. Nos. 10,508,149; 10,533,052; 10,894,830, each of which is incorporated hereby reference in its entirety. In some aspects, an anti-cancer treatment comprises an IL-7 protein and an immune checkpoint activator, wherein the immune checkpoint activator comprises an OX40 agonist (e.g., anti-OX40 antibody), LAG-3 agonist (e.g. anti-LAG-3 antibody), 4-1BB (CD137) agonist (e.g., anti-CD137 antibody), GITR agonist (e.g., anti-GITR antibody), or a combination thereof.

Where an anti-cancer treatment comprises both an IL-7 protein and an additional therapeutic agent (e.g., those described above and elsewhere in the present application), in some aspects, the IL-7 protein and the additional therapeutic agent are administered to the subject concurrently. For example, in some aspects, an IL-7 protein and an additional therapeutic agent can be administered concurrently as a single composition. In some aspects, an IL-7 protein and an additional therapeutic agent can be administered concurrently as separate compositions. In some aspects, an IL-7 protein and an additional therapeutic agent can be administered to the subject sequentially. In some aspects, the IL-7 protein is administered to the subject prior to the administration of the additional therapeutic agent. In some aspects, the IL-7 protein is administered to the subject after the administration of the additional therapeutic agent.

As demonstrated herein, in some aspects, administering an IL-7 protein in combination with an additional therapeutic agent (e.g., described herein) can result in a greater therapeutic effect as compared to administering either therapeutic agent alone. Accordingly, in some aspects, administering an anti-cancer treatment comprising both an IL-7 protein and an additional therapeutic agent (i.e., “combination treatment”) to a subject (e.g., suffering from a tumor with an unmethylated MGMT promoter) can increase an anti-tumor immune response, as compared to a corresponding subject that did not receive the combination treatment (e.g., received the IL-7 protein alone or received the additional therapeutic agent alone). In some aspects, after the administration of the combination treatment, the anti-tumor immune response in the subject is increased by at least about 10%, at least about 20%, at least about 30%, at least about 40%, at least about 50%, at least about 60%, at least about 70%, at least about 80%, at least about 90% or at least about 100%, as compared to the corresponding subject that did not receive the combination treatment. In some aspects, after the administration of the combination treatment, the anti-tumor immune response in the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, as compared to the corresponding subject that did not receive the combination treatment.

In some aspects, administering a combination treatment provided herein (e.g., comprising an IL-7 protein and an additional therapeutic agent) can reduce a tumor volume, as compared to a corresponding subject who did not receive the combination treatment (e.g., received the IL-7 protein alone or the additional therapeutic agent alone). In some aspects, after the administration of the combination treatment, the tumor volume in the subject is reduced by at least about 5%, at least about 10%, at least about 20%, at least about 30%, at least about 40%, at least about 50%, at least about 60%, at least about 70%, at least about 80%, at least about 90%, or at least about 100% as compared to the corresponding subject who did not receive the combination treatment. In some aspects, after the administration of the combination treatment, the tumor growth in the subject is reduced by at least about 5%, at least about 10%, at least about 20%, at least about 30%, at least about 40%, at least about 50%, at least about 60%, at least about 70%, at least about 80%, at least about 90%, or at least about 100% as compared to the corresponding subject who did not receive the combination treatment.

In some aspects, administering a combination treatment provided herein (e.g., comprising an IL-7 protein and an additional therapeutic agent) to a subject (e.g., suffering from a tumor with an unmethylated MGMT promoter) can increase an effector activity of a tumor-specific T cell (e.g., CD8+ T cell and/or CD4+ T cell) in the subject, as compared to a corresponding subject who did not receive the combination treatment (e.g., received the IL-7 protein alone or the additional therapeutic agent alone). In some aspects, the effector activity in the subject is increased by at least about 10%, at least about 20%, at least about 30%, at least about 40%, at least about 50%, at least about 60%, at least about 70%, at least about 80%, at least about 90%, or at least about 100%, as compared to the corresponding subject who did not receive the combination treatment. In some aspects, the effector activity in the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, as compared to the corresponding subject who did not receive the combination treatment. Non-limiting examples of effector activity of a tumor-specific T cell are provided elsewhere in the present disclosure.

In some aspects, administering a combination treatment provided herein (e.g., comprising an IL-7 protein and an additional therapeutic agent) to a subject (e.g., suffering from a tumor with an unmethylated MGMT promoter) can increase the number of tumor-specific effector T cells (e.g., CD8+ and/or CD4+), as compared to a corresponding subject who did not receive the combination treatment (e.g., received the IL-7 protein alone or the additional therapeutic agent alone). In some aspects, after the administration of the combination treatment, the number of tumor-specific effector T cells in the subject is increased by at least about 10%, at least about 20%, at least about 30%, at least about 40%, at least about 50%, at least about 60%, at least about 70%, at least about 80%, at least about 90% or at least about 100%, as compared to the corresponding subject who did not receive the combination treatment. In some aspects, the number of tumor-specific effector T cells in the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, as compared to the corresponding subject who did not receive the combination treatment.

In some aspects, administering a combination treatment provided herein (e.g., comprising an IL-7 protein and an additional therapeutic agent) to a subject (e.g., suffering from a tumor with an unmethylated MGMT promoter) can increase the proliferation of the tumor-specific effector T cells in the subject, as compared to a corresponding subject who did not receive the combination treatment (e.g., received the IL-7 protein alone or the additional therapeutic agent alone). In some aspects, after the administration of the combination treatment, the proliferation of the tumor-specific effector T cells is increased by at least about 10%, at least about 20%, at least about 30%, at least about 40%, at least about 50%, at least about 60%, at least about 70%, at least about 80%, at least about 90% or at least about 100%, as compared to that of the corresponding subject who did not receive the combination treatment. In some aspects, after the administration of the combination treatment, the proliferation of the tumor-specific effector T cells is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, as compared to that of the corresponding subject who did not receive the combination treatment.

In some aspects, administering a combination treatment provided herein (e.g., comprising an IL-7 protein and an additional therapeutic agent) to a subject (e.g., suffering from a tumor with an unmethylated MGMT promoter) can increase the ability of the tumor-specific effector T cells to survive and/or persist in the subject, as compared to a corresponding subject who did not receive the combination treatment (e.g., received the IL-7 protein alone or the additional therapeutic agent alone). In some aspects, after the administration of the combination treatment, the ability of the tumor-specific effector T cells to survive and/or persist in the subject is increased by at least about 10%, at least about 20%, at least about 30%, at least about 40%, at least about 50%, at least about 60%, at least about 70%, at least about 80%, at least about 90% or at least about 100%, as compared to the corresponding subject who did not receive the combination treatment. In some aspects, after the administration of the combination treatment, the ability of the tumor-specific effector T cells to survive and/or persist in the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, as compared to the corresponding subject who did not receive the combination treatment. In some aspects, after the administration of the combination treatment, both the proliferation and survival/persistence of the tumor-specific effector T cells is increased in the subject, as compared to the corresponding subject who did not receive the combination treatment.

In some aspects, administering a combination treatment provided herein (e.g., comprising an IL-7 protein and an additional therapeutic agent) to a subject (e.g., suffering from a tumor with an unmethylated MGMT promoter) can increase the number of TILs in a tumor of the subject, as compared to a corresponding subject who did not receive the combination treatment (e.g., received the IL-7 protein alone or the additional therapeutic agent alone). In some aspects, after the administration of the combination treatment, the number of TILs in a tumor of the subject is increased by at least about 10%, at least about 20%, at least about 30%, at least about 40%, at least about 50%, at least about 60%, at least about 70%, at least about 80%, at least about 90% or at least about 100%, as compared to the corresponding subject who did not receive the combination treatment. In some aspects, after the administration of the combination treatment, the number of TILs in a tumor of the subject is increase by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, as compared to the corresponding subject who did not receive the combination treatment.

In some aspects, administering a combination treatment provided herein (e.g., comprising an IL-7 protein and an additional therapeutic agent) to a subject (e.g., suffering from a tumor with an unmethylated MGMT promoter) can increase the absolute lymphocyte count in the subject, as compared to a corresponding subject who did not receive the combination treatment (e.g., received the IL-7 protein alone or the additional therapeutic agent alone). In some aspects, after the administration of the combination treatment, the absolute lymphocyte count in the subject is increased by at least about 10%, at least about 20%, at least about 30%, at least about 40%, at least about 50%, at least about 60%, at least about 70%, at least about 80%, at least about 90% or at least about 100%, as compared to the corresponding subject who did not receive the combination treatment. In some aspects, after the administration of the combination treatment, the absolute lymphocyte count in the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, as compared to the corresponding subject who did not receive the combination treatment.

In some aspects, administering a combination treatment provided herein (e.g., comprising an IL-7 protein and an additional therapeutic agent) to a subject (e.g., suffering from a tumor with an unmethylated MGMT tumor) can increase the duration of overall survival of the subject, as compared to a corresponding subject who did not receive the combination treatment (e.g., received the IL-7 protein alone or the additional therapeutic agent alone). In some aspects, after the administration of the combination treatment, the duration of overall survival of the subject is increased by at least about 10%, at least about 20%, at least about 30%, at least about 40%, at least about 50%, at least about 60%, at least about 70%, at least about 80%, at least about 90% or at least about 100%, as compared to the corresponding subject who did not receive the combination treatment. In some aspects, after the administration of the combination treatment, the duration of overall survival of the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, as compared to the corresponding subject who did not receive the combination treatment.

In some aspects, administering a combination treatment provided herein (e.g., comprising an IL-7 protein and an additional therapeutic agent) to a subject (e.g., suffering from a tumor with an unmethylated MGMT promoter) can increase the progression-free survival of the subject, as compared to a corresponding subject who did not receive the combination treatment (e.g., received the IL-7 protein alone or the additional therapeutic agent alone). In some aspects, after the administration of the combination treatment, the progression-free survival of the subject is increased by at least about 10%, at least about 20%, at least about 30%, at least about 40%, at least about 50%, at least about 60%, at least about 70%, at least about 80%, at least about 90% or at least about 100%, as compared to the corresponding subject who did not receive the combination treatment. In some aspects, after the administration of the combination treatment, the progression-free survival of the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, as compared to the corresponding subject who did not receive the combination treatment.

In some aspects, any of the therapeutic effects described herein can occur under lymphopenic conditions. For instance, many cancer patients (e.g., suffering from brain cancer, e.g., GBM) exhibit edema (e.g., brain edema). Therefore, to treat such symptoms, a steroid (e.g., corticosteroid) can be administered to the cancer patients. While it may be useful in alleviating certain symptoms, steroids are known to decrease the activity and functionality of the immune system, including causing lymphopenia when administered to humans. See, e.g., Patrick Roth et al., Neurooncol Pract 2 (1): 6-12 (March 2015). For instance, in some aspects, an anti-cancer treatment described herein (e.g., comprising an IL-7 protein alone or in combination with an additional therapeutic agent) can enhance an anti-tumor immune response in a subject that was treated with a steroid (such as that which causes lymphopenia). In some aspects, the anti-tumor immune response in the subject is increased by at least about 10%, at least about 20%, at least about 30%, at least about 40%, at least about 50%, at least about 60%, at least about 70%, at least about 80%, at least about 90% or at least about 100%, as compared to a corresponding subject (i.e., that was treated with a steroid) that did not receive the anti-cancer treatment. In some aspects, the anti-tumor immune response in the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, as compared to a corresponding subject (i.e., that was treated with a steroid) that did not receive the anti-cancer treatment.

In some aspects, an anti-cancer treatment described herein (e.g., comprising an IL-7 protein alone or in combination with an additional therapeutic agent) can reduce a tumor volume and/or tumor growth in a subject that was treated with a steroid (such as that which causes lymphopenia). In some aspects, the tumor volume in the subject is reduced by at least about 5%, at least about 10%, at least about 20%, at least about 30%, at least about 40%, at least about 50%, at least about 60%, at least about 70%, at least about 80%, at least about 90%, or at least about 100%, as compared to a corresponding subject (i.e., that was treated with a steroid) who did not receive the anti-cancer treatment. In some aspects, the tumor growth in the subject is reduced by at least about 5%, at least about 10%, at least about 20%, at least about 30%, at least about 40%, at least about 50%, at least about 60%, at least about 70%, at least about 80%, at least about 90%, or at least about 100% as compared to a corresponding subject (i.e., that was treated with a steroid) who did not receive the anti-cancer treatment.

In some aspects, an anti-cancer treatment described herein (e.g., comprising an IL-7 protein alone or in combination with an additional therapeutic agent) can increase an effector activity of a tumor-specific T cell (e.g., CD8+ T cell and/or CD4+ T cell) in a subject that was treated with a steroid (such as that which causes lymphopenia). In some aspects, the effector activity in the subject is increased by at least about 10%, at least about 20%, at least about 30%, at least about 40%, at least about 50%, at least about 60%, at least about 70%, at least about 80%, at least about 90%, or at least about 100%, as compared to a corresponding subject (i.e., that was treated with a steroid) who did not receive the anti-cancer treatment. In some aspects, the effector activity in the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, as compared to a corresponding subject (i.e., that was treated with a steroid) who did not receive the anti-cancer treatment.

In some aspects, an anti-cancer treatment described herein (e.g., comprising an IL-7 protein alone or in combination with an additional therapeutic agent) can increase the number of tumor-specific effector T cells (e.g., CD8+ and/or CD4+) in a subject that was treated with a steroid (such as that which causes lymphopenia). In some aspects, the number of tumor-specific effector T cells in the subject is increased by at least about 10%, at least about 20%, at least about 30%, at least about 40%, at least about 50%, at least about 60%, at least about 70%, at least about 80%, at least about 90% or at least about 100%, as compared to a corresponding subject (i.e., that was treated with a steroid) who did not receive the anti-cancer treatment. In some aspects, the number of tumor-specific effector T cells in the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, as compared to a corresponding subject (i.e., that was treated with a steroid) who did not receive the anti-cancer treatment.

In some aspects, an anti-cancer treatment described herein (e.g., comprising an IL-7 protein alone or in combination with an additional therapeutic agent) can increase the proliferation of the tumor-specific effector T cells in a subject that was treated with a steroid (such as that which causes lymphopenia). In some aspects, the proliferation of the tumor-specific effector T cells is increased by at least about 10%, at least about 20%, at least about 30%, at least about 40%, at least about 50%, at least about 60%, at least about 70%, at least about 80%, at least about 90% or at least about 100%, as compared to that of a corresponding subject (i.e., that was treated with a steroid) who did not receive the anti-cancer treatment. In some aspects, the proliferation of the tumor-specific effector T cells is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, as compared to that of a corresponding subject (i.e., that was treated with a steroid) who did not receive the anti-cancer treatment.

In some aspects, an anti-cancer treatment described herein (e.g., comprising an IL-7 protein alone or in combination with an additional therapeutic agent) can increase the ability of the tumor-specific effector T cells to survive and/or persist in a subject that was treated with a steroid (such as that which causes lymphopenia). In some aspects, the ability of the tumor-specific effector T cells to survive and/or persist in the subject is increased by at least about 10%, at least about 20%, at least about 30%, at least about 40%, at least about 50%, at least about 60%, at least about 70%, at least about 80%, at least about 90% or at least about 100%, as compared to a corresponding subject (i.e., that was treated with a steroid) who did not receive the anti-cancer treatment. In some aspects, the ability of the tumor-specific effector T cells to survive and/or persist in the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, as compared to a corresponding subject (i.e., that was treated with a steroid) who did not receive the anti-cancer treatment. In some aspects, both the proliferation and survival/persistence of the tumor-specific effector T cells is increased in the subject, as compared to a corresponding subject (i.e., that was treated with a steroid) who did not receive the anti-cancer treatment.

In some aspects, an anti-cancer treatment described herein (e.g., comprising an IL-7 protein alone or in combination with an additional therapeutic agent) can increase the number of TILs in a subject that was treated with a steroid (such as that which causes lymphopenia). In some aspects, the number of TILs in a tumor of the subject is increased by at least about 10%, at least about 20%, at least about 30%, at least about 40%, at least about 50%, at least about 60%, at least about 70%, at least about 80%, at least about 90% or at least about 100%, as compared to a corresponding subject (i.e., that was treated with a steroid) who did not receive the combination treatment. In some aspects, the number of TILs in a tumor of the subject is increase by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, as compared to a corresponding subject (i.e., that was treated with a steroid) who did not receive the combination treatment.

In some aspects, an anti-cancer treatment described herein (e.g., comprising an IL-7 protein alone or in combination with an additional therapeutic agent) can increase the absolute lymphocyte count in a subject that was treated with a steroid (such as that which causes lymphopenia). In some aspects, the absolute lymphocyte count in the subject is increased by at least about 10%, at least about 20%, at least about 30%, at least about 40%, at least about 50%, at least about 60%, at least about 70%, at least about 80%, at least about 90% or at least about 100%, as compared to a corresponding subject (i.e., that was treated with a steroid) who did not receive the anti-cancer treatment. In some aspects, the absolute lymphocyte count in the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, as compared to a corresponding subject (i.e., that was treated with a steroid) who did not receive the anti-cancer treatment.

In some aspects, anti-cancer treatment described herein (e.g., comprising an IL-7 protein alone or in combination with an additional therapeutic agent) can increase the duration of overall survival of a subject that was treated with a steroid (such as that which causes lymphopenia). In some aspects, the duration of overall survival of the subject is increased by at least about 10%, at least about 20%, at least about 30%, at least about 40%, at least about 50%, at least about 60%, at least about 70%, at least about 80%, at least about 90% or at least about 100%, as compared to a corresponding subject (i.e., that was treated with a steroid) who did not receive the anti-cancer treatment. In some aspects, the duration of overall survival of the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, as compared to a corresponding subject (i.e., that was treated with a steroid) who did not receive the anti-cancer treatment.

In some aspects, anti-cancer treatment described herein (e.g., comprising an IL-7 protein alone or in combination with an additional therapeutic agent) can increase the progression-free survival of a subject that was treated with a steroid (such as that which causes lymphopenia). In some aspects, the progression-free survival of the subject is increased by at least about 10%, at least about 20%, at least about 30%, at least about 40%, at least about 50%, at least about 60%, at least about 70%, at least about 80%, at least about 90% or at least about 100%, as compared to a corresponding subject (i.e., that was treated with a steroid) who did not receive the anti-cancer treatment. In some aspects, the progression-free survival of the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, as compared to a corresponding subject (i.e., that was treated with a steroid) who did not receive the anti-cancer treatment.

In some aspects, an anti-cancer treatment described herein (e.g., comprising an IL-7 protein alone or in combination with an additional therapeutic agent) can be used with one or more additional cancer therapy. Non-limiting examples of such additional cancer therapy include: chemotherapy drugs, small molecule drugs, radiation, immune checkpoint inhibitor, immune checkpoint activator, or a combination thereof. Accordingly, in some aspects, provided herein is a method of treating a tumor (e.g., comprising an unmethylated MGMT tumor) in a subject in need thereof, comprising administering to the subject (i) an anti-cancer treatment, which comprises an IL-7 protein alone or in combination with an additional therapeutic agent (e.g., chemotherapy, e.g., TMZ) and (ii) an additional cancer therapy. Non-limiting examples of such combinations can include: a therapy that enhances tumor antigen presentation (e.g., dendritic cell vaccine, GM-CSF secreting cellular vaccines, CpG oligonucleotides, imiquimod); a therapy that inhibits negative immune regulation e.g., by inhibiting CTLA-4 and/or PD1/PD-L1/PD-L2 pathway and/or depleting or blocking Tregs or other immune suppressing cells (e.g., myeloid-derived suppressor cells); a therapy that stimulates positive immune regulation, e.g., with agonists that stimulate the CD-137, OX-40, and/or CD40 or GITR pathway and/or stimulate T cell effector function; a therapy that increases systemically the frequency of anti-tumor T cells; a therapy that depletes or inhibits Tregs, such as Tregs in the tumor, e.g., using an antagonist of CD25 (e.g., daclizumab) or by ex vivo anti-CD25 bead depletion; a therapy that impacts the function of suppressor myeloid cells in the tumor; a therapy that enhances immunogenicity of tumor cells (e.g., anthracyclines); adoptive T cell or NK cell transfer including genetically modified cells, e.g., cells modified by chimeric antigen receptors (CAR-T therapy); a therapy that inhibits a metabolic enzyme such as indoleamine dioxigenase (IDO), dioxigenase, arginase, or nitric oxide synthetase; a therapy that reverses/prevents T cell anergy or exhaustion; a therapy that triggers an innate immune activation and/or inflammation at a tumor site; administration of immune stimulatory cytokines; or blocking of immuno repressive cytokines.

### 2. Tumors

As will be apparent from the present disclosure, the anti-cancer treatments provided herein (e.g., comprising an IL-7 protein alone or in combination with an additional therapeutic agent) can be useful in treating a wide range of cancers. As demonstrated and described herein, the anti-cancer treatments provided herein are particularly effective in treating a tumor having a MGMT promoter that is unmethylated. It has been shown that methylation of the O⁶-methylguanine DNA methyltransferase (MGMT) promoter has emerged as strong prognostic factor in the therapy of GBM. Feldheim et al., Cancers (Basel) 11 (12): 1837 (December 2019). For example, individuals with GBM having methylated MGMT promoter are associated with improved response to treatment (e.g., chemotherapy with temoozolomide) and exhibit longer overall survival. Alnahhas et al., Neurooncol Adv 2 (1): vdaa082 (January-December 2020). Unfortunately, where the MGMT promoter is unmethylated, individuals with such GBM are less responsive to treatment and have much poorer prognosis.

Accordingly, a target tumor that can be treated with the present disclosure comprises any tumor with an unmethylated MGMT promoter. In some aspects, a tumor with an unmethylated MGMT promoter comprises a glioma. As used herein, the term “glioma” refers to a type of tumor that occurs within the brain and spinal cord (i.e., arises from glial cells or their precursors of the brain or spinal cord). There are four main types of glioma: (1) astrocytoma (which is the most common type of glioma in both adults and children), (2) ependymoma, (3) oligodendroglioma, and (4) mixed glioma. Gliomas can be classified according to their location: infratentorial (i.e., located in the lower part of the brain, found mostly children patients) or supratentorial (i.e., located in the upper part of the brain, found mostly adults patients).

Gliomas can be further categorized according to their grade, which is determined by pathologic evaluation of the tumor. The World Health Organization (WHO) has developed a grading system, from Grade I gliomas, which tend to be the least aggressive to Grade IV gliomas, which tend to be the most aggressive and malignant. Non-limiting examples of low grade gliomas include: pilocytic astrocytoma (also called juvenile pilocytic astrocytoma), fibrillary astrocytoma, pleomorphic xantroastrocytomoa, and desembryoplastic neuroepithelial tumor. Non-limiting examples of high-grade gliomas include: anaplastic astrocytoma (Grade III) and glioblastoma multiforme (Grade IV). Of the different gliomas, Grade IV gliomas have the worst prognosis, with an average survival time of about 12 months or less.

In some aspects, a glioma that can be treated with the present disclosure comprises a high-grade glioma. In some aspects, the glioma comprises a Grade III glioma. In some aspects, the glioma comprises a Grade IV glioma. Accordingly, in some aspects, provided herein is a method of treating a tumor with an unmethylated MGMT promoter in a subject in need thereof, comprising administering to the subject an anti-cancer treatment, wherein the anti-cancer treatment comprises an interleukin-7 (IL-7) protein, and wherein the tumor comprises a high-grade glioma. Non-limiting examples of such high-grade glioma includes: a glioblastoma multiforme (GBM), anaplastic astrocytoma, or both. In some aspects, provided herein is a method of treating a GBM in a subject in need thereof, comprising administering to the subject an anti-cancer treatment which comprises an IL-7 protein, wherein the GBM has an unmethylated MGMT promoter. In some aspects, provided herein is a method of treating a GBM in a subject in need thereof, comprising administering to the subject an anti-cancer treatment which comprises an IL-7 protein and an additional therapeutic agent (e.g., those described herein), wherein the GBM has an unmethylated MGMT promoter. While the present disclosure is generally directed to treating tumors with an unmethylated MGMT promoter, it will be apparent to those skilled in the art that the disclosures provided herein could also be useful in treating other types of tumors. For example, in some aspects, the present disclosure can be useful in treating a high risk Grade II glioma.

In some aspects, a tumor that can be treated with the present disclosure comprises an unmethylated MGMT promoter and is metastatic, unresectable, refractory (e.g., to previous cancer therapy, e.g., immunotherapy, e.g., with an immune checkpoint inhibitor), and/or recurrent. In some aspects, the tumor comprises an unmethylated MGMT promoter and is metastatic. In some aspects, the tumor comprises an unmethylated MGMT promoter and is unresectable. In some aspects, the tumor comprises an unmethylated MGMT promoter and is refractory. In some aspects, the tumor comprise an unmethylated MGMT promoter and is recurrent. In some aspects, the tumor is newly diagnosed (i.e., has not previously been treated with an anti-cancer treatment).

### 3. Target Subjects

Unless indicated otherwise, any subject suffering from a tumor with an unmethylated MGMT promoter can be treated with the present disclosure. In some aspects, such a subject comprises a nonhuman animal, e.g., such as a rat or a mouse. In some aspects, the subject that can be treated with the present disclosure comprises a human.

In some aspects, a subject that can be treated with the present disclosure has been newly diagnosed with a tumor described herein (e.g., comprising an unmethylated MGMT promoter). Accordingly, in some aspects, the subject is treatment-naïve (i.e., has never received a prior cancer treatment). In some aspects, the subject has progressed on other cancer treatments. Accordingly, in some aspects, a subject that can be treated with the present disclosure has previously received a cancer treatment. In some aspects, the prior cancer treatment comprised an immunotherapy (e.g., with an anti-PD-1 antibody). In some aspects, the prior cancer treatment comprised a chemotherapy. In some aspects, the chemotherapy comprises a platinum-based therapy. In some aspects, the platinum-based therapy comprises a platinum-based antineoplastic selected from the group consisting of cisplatin, carboplatin, oxaliplatin, nedaplatin, triplatin tetranitrate, phenanthriplatin, picoplatin, satraplatin, and any combination thereof.

In some aspects, a subject that can be treated with the present disclosure suffers from a Grade III glioma, as determined under the WHO classification system. In some aspects, the subject suffers from a Grade IV glioma, as determined under the WHO classification system. In some aspects, the subject suffers from a high risk Grade II glioma, as determined under the WHO classification system. As is apparent from the present disclosure, the subject that can be treated with the present disclosure suffers from a glioma with an unmethylated MGMT promoter. In some aspects, the subject has received one or more post-operative treatments. Non-limiting examples of such post-operative treatments include: radiation, chemotherapy (e.g., TMZ or carmustine (also known as GLIADEL Wafer)), glucocorticoid therapy, tumor treating fields (TTF) (OPTUNE®), or combinations thereof. In some aspects, the post-operative treatment comprised both radiation and TMZ. In some aspects, a subject that can be treated with the present disclosure has adequate organ and marrow function defined as follows: (1) absolute neutrophil count≥1,000/mcL; (2) platelets≥75,000/mcL; (3) hemoglobin≥8 g/dL; (4) total bilirubin≤3.0 × institutional upper limit of normal; (5) AST (SGOT)/ALT (SGPT)≤3.0 × institutional upper limit of normal. In some aspects, the subject that can be treated with the present disclosure has an absolute lymphocyte count (ALC) of 600 mcL. In some aspects, a subject that can be treated with the present disclosure does not have an active viral infection. In some aspects, a subject that can be treated with the present disclosure does not have an active autoimmune disease or syndrome. In some aspects, a subject that can be treated with the present disclosure has not received a live attenuated vaccine with 30 days before the administration of the first dose of the anti-cancer treatment described herein (i.e., comprising an IL-7 protein alone or in combination with an additional therapeutic agent).

### 4. Dosing Regimen

As is apparent from the present disclosure, methods provided herein (e.g., treating a tumor with an unmethylated MGMT promoter) comprises administering to a subject one or more doses of an IL-7 protein. In some aspects, the unit dose (e.g., for human use) of an IL-7 protein disclosed herein can be in the range of about 0.001 mg/kg to about 10 mg/kg. In some aspects, the unit dose of an IL-7 protein is in the range of about 0.01 mg/kg to about 2 mg/kg. In some aspects, the unit dose is in the range of about 0.02 mg/kg to about 1 mg/kg.

In some aspects, an IL-7 protein disclosed herein can be administered to a subject at a weight-based dose. In some aspects, an IL-7 protein can be administered at a weight-based dose between about 20 μg/kg and about 600 μg/kg. In some aspects, an IL-7 protein of the present disclosure can be administered at a weight-based dose of about 20 μg/kg, about 60 μg/kg, about 120 μg/kg, about 240 μg/kg, about 360 μg/kg, about 480 μg/kg, or about 600 μg/kg. In some aspects, the IL-7 protein is administered to the subject at a dose of 60 μg/kg. In some aspects, the IL-7 protein is administered to the subject at a dose of 120 μg/kg. In some aspects, the IL-7 protein is administered to the subject at a dose of 240 μg/kg.

In some aspects, an IL-7 protein disclosed herein can be administered to a subject at a dose greater than about 30 μg/kg, greater than about 60 μg/kg, greater than about 90 μg/kg, greater than about 120 μg/kg, greater than about 150 μg/kg, greater than about 180 μg/kg, greater than about 210 μg/kg, greater than about 240 μg/kg, greater than about 270 μg/kg, greater than about 300 μg/kg, greater than about 400 μg/kg, greater than about 500 μg/kg, greater than about 600 μg/kg. In some aspects, an IL-7 protein is administered to a subject at a dose greater than about 600 μg/kg, greater than about 700 μg/kg, greater than about 800 μg/kg, greater than about 900 μg/kg, greater than about 1,000 μg/kg, greater than about 1,100 μg/kg, greater than about 1,200 μg/kg, greater than about 1,300 μg/kg, greater than about 1,400 μg/kg, greater than about 1,500 μg/kg, greater than about 1,600 μg/kg, greater than about 1,700 μg/kg, greater than about 1,800 μg/kg, greater than about 1,900 μg/kg, or greater than about 2,000 μg/kg.

In some aspects, an IL-7 protein of the present disclosure is administered at a dose of between about 60 μg/kg and about 1,200 μg/kg, between about 120 μg/kg and about 1,200 μg/kg, between about 240 μg/kg and about 1,200 μg/kg, between about 540 μg/kg and about 1,200 μg/kg, between 610 μg/kg and about 1,200 μg/kg, between 650 μg/kg and about 1,200 μg/kg, between about 700 μg/kg and about 1,200 μg/kg, between about 750 μg/kg and about 1,200 μg/kg, between about 800 μg/kg and about 1,200 μg/kg, between about 850 μg/kg and about 1,200 μg/kg, between about 900 μg/kg and about 1,200 μg/kg, between about 950 μg/kg and about 1,200 μg/kg, between about 1,000 μg/kg and about 1,200 μg/kg, between about 1,050 μg/kg and about 1,200 μg/kg, between about 1,100 μg/kg and about 1,200 μg/kg, between about 1,200 μg/kg and about 2,000 μg/kg, between about 1,300 μg/kg and about 2,000 μg/kg, between about 1,500 μg/kg and about 2,000 μg/kg, between about 1,700 μg/kg and about 2,000 μg/kg, between about 610 μg/kg and about 1,000 μg/kg, between about 650 μg/kg and about 1,000 μg/kg, between about 700 μg/kg and about 1,000 μg/kg, between about 750 μg/kg and about 1,000 μg/kg, between about 800 μg/kg and about 1,000 μg/kg, between about 850 μg/kg and about 1,000 μg/kg, between about 900 μg/kg and about 1,000 μg/kg, or between about 950 μg/kg and about 1,000 μg/kg.

In some aspects, an IL-7 protein is administered at a dose of between about 60 μg/kg and about 1,200 μg/kg. In some aspects, an IL-7 protein is administered at a dose of between about 120 μg/kg and about 1,200 μg/kg. In some aspects, an IL-7 protein is administered at a dose of between about 240 μg/kg and about 1,200 μg/kg. In some aspects, an IL-7 protein is administered at a dose of between about 540 μg/kg and about 1,200 μg/kg. In some aspects, an IL-7 protein of the present disclosure is administered at a dose of between 610 μg/kg and about 1,200 μg/kg. In some aspects, an IL-7 protein is administered at a dose of between 650 μg/kg and about 1,200 μg/kg. In some aspects, an IL-7 protein is administered at a dose of between about 700 μg/kg and about 1,200 μg/kg. In some aspects, an IL-7 protein is administered at a dose of between about 750 μg/kg and about 1,200 μg/kg. In some aspects, an IL-7 protein is administered at a dose of between about 800 μg/kg and about 1,200 μg/kg. In some aspects, an IL-7 protein is administered at a dose of between about 850 μg/kg and about 1,200 μg/kg. In some aspects, an IL-7 protein is administered at a dose of between about 900 μg/kg and about 1,200 μg/kg. In some aspects, an IL-7 protein is administered at a dose of between about 950 μg/kg and about 1,200 μg/kg. In some aspects, an IL-7 protein disclosed herein is administered at a dose of between about 1,000 μg/kg and about 1,200 μg/kg. In some aspects, an IL-7 protein is administered at a dose of between about 1,050 μg/kg and about 1,200 μg/kg. In some aspects, an IL-7 protein is administered at a dose of between about 1,100 μg/kg and about 1,200 μg/kg. In some aspects, an IL-7 protein is administered at a dose of between about 1,200 μg/kg and about 2,000 μg/kg. In some aspects, an IL-7 protein is administered at a dose of between about 1,300 μg/kg and about 2,000 μg/kg. In some aspects, an IL-7 protein is administered at a dose of between about 1,500 μg/kg and about 2,000 μg/kg. In some aspects, an IL-7 protein is administered at a dose of between about 1,700 μg/kg and about 2,000 μg/kg. In some aspects, an IL-7 protein is administered at a dose of between about 610 μg/kg and about 1,000 μg/kg. In some aspects, an IL-7 protein is administered at a dose of between about 650 μg/kg and about 1,000 μg/kg. In some aspects, an IL-7 protein is administered at a dose of between about 700 μg/kg and about 1,000 μg/kg. In some aspects, an IL-7 protein is administered at a dose of between about 750 μg/kg and about 1,000 μg/kg. In some aspects, an IL-7 protein is administered at a dose of between about 800 μg/kg and about 1,000 μg/kg. In some aspects, an IL-7 protein is administered at a dose of between about 850 μg/kg and about 1,000 μg/kg. In some aspects, an IL-7 protein of the present disclosure is administered at a dose of between about 900 μg/kg and about 1,000 μg/kg. In some aspects, an IL-7 protein is administered at a dose of between about 950 μg/kg and about 1,000 μg/kg.

In some aspects, an IL-7 protein is administered at a dose of between about 700 μg/kg and about 900 μg/kg, between about 750 μg/kg and about 950 μg/kg, between about 700 μg/kg and about 850 μg/kg, between about 750 μg/kg and about 850 μg/kg, between about 700 μg/kg and about 800 μg/kg, between about 800 μg/kg and about 900 μg/kg, between about 750 μg/kg and about 850 μg/kg, or between about 850 μg/kg and about 950 μg/kg. In some aspects, an IL-7 protein is administered at a dose of between about 700 μg/kg and about 900 μg/kg. In some aspects, an IL-7 protein is administered at a dose of between about 750 μg/kg and about 950 μg/kg. In some aspects, an IL-7 protein is administered at a dose of between about 700 μg/kg and about 850 μg/kg. In some aspects, an IL-7 protein is administered at a dose of between about 750 μg/kg and about 850 μg/kg. In some aspects, an IL-7 protein is administered at a dose of between about 700 μg/kg and about 800 μg/kg. In some aspects, an IL-7 protein is administered at a dose of between about 800 μg/kg and about 900 μg/kg. In some aspects, an IL-7 protein is administered at a dose of between about 750 μg/kg and about 850 μg/kg. In some aspects, an IL-7 protein is administered at a dose of between about 850 μg/kg and about 950 μg/kg.

In some aspects, an IL-7 protein is administered at a dose of about 30 μg/kg, about 60 μg/kg, about 90 μg/kg, about 120 μg/kg, about 150 μg/kg, about 180 μg/kg, about 210 μg/kg, about 240 μg/kg, about 270 μg/kg, about 300 μg/kg, about 330 μg/kg, about 360 μg/kg, about 390 μg/kg, about 420 μg/kg, about 450 μg/kg, about 480 μg/kg, about 510 μg/kg, about 540 μg/kg, about 570 μg/kg, about 600 μg/kg, about 630 μg/kg, about 650 μg/kg, about 680 μg/kg, about 700 μg/kg, about 720 μg/kg, about 740 μg/kg, about 750 μg/kg, about 760 μg/kg, about 780 μg/kg, about 800 μg/kg, about 820 μg/kg, about 840 μg/kg, about 850 μg/kg, about 860 μg/kg, about 880 μg/kg, about 900 μg/kg, about 920 μg/kg, about 940 μg/kg, about 950 μg/kg, about 960 μg/kg, about 980 μg/kg, about 1,000 μg/kg, about 1,020 μg/kg, about 1,020 μg/kg, about 1,040 μg/kg, about 1,060 μg/kg, about 1,080 μg/kg, about 1,100 μg/kg, about 1,120 μg/kg, about 1,140 μg/kg, about 1,160 μg/kg, about 1,180 μg/kg, about 1200 μg/kg, about 1,220 μg/kg, about 1,240 μg/kg, about 1,260 μg/kg, about 1,280 μg/kg, about 1,300 μg/kg, about 1,320 μg/kg, about 1,340 μg/kg, about 1,360 μg/kg, about 1,380 μg/kg, about 1,400 μg/kg, about 1,420 μg/kg, about 1,440 μg/kg, about 1,460 μg/kg, about 1,480 μg/kg, about 1,500 μg/kg, about 1,520 μg/kg, about 1,540 μg/kg, about 1,560 μg/kg, about 1,580 μg/kg, about 1,600 μg/kg, about 1,620 μg/kg, about 1,640 μg/kg, about 1,660 μg/kg, about 1,680 μg/kg, about 1,700 μg/kg, about 1,720 μg/kg, about 1,740 μg/kg, about 1,760 μg/kg, about 1,780 μg/kg, about 1,800 μg/kg, about 1,820 μg/kg, about 1,840 μg/kg, about 1,860 μg/kg, about 1,880 μg/kg, about 1,900 μg/kg, about 1,920 μg/kg, about 1,940 μg/kg, about 1,960 μg/kg, about 1,980 μg/kg, or about 2,000 μg/kg.

In some aspects, an IL-7 protein is administered at a dose of about 30 μg/kg. In some aspects, an IL-7 protein is administered at a dose of about 60 μg/kg. In some aspects, an IL-7 protein is administered at a dose of about 90 μg/kg. In some aspects, an IL-7 protein is administered at a dose of about 120 μg/kg. In some aspects, an IL-7 protein is administered at a dose of about 150 μg/kg. In some aspects, an IL-7 protein is administered at a dose of about 180 μg/kg. In some aspects, an IL-7 protein is administered at a dose of about 210 μg/kg. In some aspects, an IL-7 protein is administered at a dose of about 240 μg/kg. In some aspects, an IL-7 protein is administered at a dose of about 270 μg/kg. In some aspects, an IL-7 protein is administered at a dose of about 300 μg/kg. In some aspects, an IL-7 protein is administered at a dose of about 330 μg/kg. In some aspects, an IL-7 protein is administered at a dose of about 360 μg/kg. In some aspects, an IL-7 protein is administered at a dose of about 390 μg/kg. In some aspects, an IL-7 protein is administered at a dose of about 420 μg/kg. In some aspects, an IL-7 protein is administered at a dose of about 450 μg/kg. In some aspects, an IL-7 protein is administered at a dose of about 480 μg/kg. In some aspects, an IL-7 protein is administered at a dose of about 510 μg/kg. In some aspects, an IL-7 protein is administered at a dose of about 540 μg/kg. In some aspects, an IL-7 protein is administered at a dose of about 570 μg/kg. In some aspects, an IL-7 protein is administered at a dose of about 600 μg/kg. In some aspects, an IL-7 protein is administered at a dose of about 630 μg/kg. In some aspects, an IL-7 protein is administered at a dose of about 650 μg/kg. In some aspects, an IL-7 protein disclosed herein is administered at a dose of about 680 μg/kg. In some aspects, an IL-7 protein is administered at a dose of about 700 μg/kg. In some aspects, an IL-7 protein is administered at a dose of about 720 μg/kg. In some aspects, an IL-7 protein is administered at a dose of about 740 μg/kg. In some aspects, an IL-7 protein is administered at a dose of about 750 μg/kg. In some aspects, an IL-7 protein is administered at a dose of about 760 μg/kg. In some aspects, an IL-7 protein is administered at a dose of about 780 μg/kg. In some aspects, an IL-7 protein is administered at a dose of about 800 μg/kg. In some aspects, an IL-7 protein is administered at a dose of about 820 μg/kg. In some aspects, an IL-7 protein is administered at a dose of about 840 μg/kg. In some aspects, an IL-7 protein is administered at a dose of about 850 μg/kg. In some aspects, an IL-7 protein is administered at a dose of about 860 μg/kg. In some aspects, an IL-7 protein is administered at a dose of about 880 μg/kg. In some aspects, an IL-7 protein is administered at a dose of about 900 μg/kg. In some aspects, an IL-7 protein is administered at a dose of about 920 μg/kg. In some aspects, an IL-7 protein is administered at a dose of about 940 μg/kg. In some aspects, an IL-7 protein is administered at a dose of about 950 μg/kg. In some aspects, an IL-7 protein is administered at a dose of about 960 μg/kg. In some aspects, an IL-7 protein is administered at a dose of about 980 μg/kg. In some aspects, an IL-7 protein is administered at a dose of about 1,000 μg/kg. In some aspects, an IL-7 protein is administered at a dose of about 1,020 μg/kg. In some aspects, an IL-7 protein is administered at a dose of about 1,040 μg/kg. In some aspects, an IL-7 protein is administered at a dose of about 1,060 μg/kg. In some aspects, an IL-7 protein is administered at a dose of about 1,080 μg/kg. In some aspects, an IL-7 protein is administered at a dose of about 1,100 μg/kg. In some aspects, an IL-7 protein is administered at a dose of about 1,120 μg/kg. In some aspects, an IL-7 protein is administered at a dose of about 1,140 μg/kg. In some aspects, an IL-7 protein is administered at a dose of about 1,160 μg/kg. In some aspects, an IL-7 protein is administered at a dose of about 1,180 μg/kg. In some aspects, an IL-7 protein is administered at a dose of about 1,200 μg/kg. In some aspects, an IL-7 protein is administered at a dose of about 1,220 μg/kg. In some aspects, an IL-7 protein is administered at a dose of about 1,240 μg/kg. In some aspects, an IL-7 protein is administered at a dose of about 1,260 μg/kg. In some aspects, an IL-7 protein is administered at a dose of about 1,280 μg/kg. In some aspects, an IL-7 protein is administered at a dose of about 1,300 μg/kg. In some aspects, an IL-7 protein is administered at a dose of about 1,320 μg/kg. In some aspects, an IL-7 protein is administered at a dose of about 1,340 μg/kg. In some aspects, an IL-7 protein is administered at a dose of about 1,360 μg/kg. In some aspects, an IL-7 protein is administered at a dose of about 1,380 μg/kg. In some aspects, an IL-7 protein is administered at a dose of about 1,400 μg/kg. In some aspects, an IL-7 protein is administered at a dose of about 1,420 μg/kg. In some aspects, an IL-7 protein is administered at a dose of about 1,440 μg/kg. In some aspects, an IL-7 protein is administered at a dose of about 1,460 μg/kg. In some aspects, an IL-7 protein is administered at a dose of about 1,480 μg/kg. In some aspects, an IL-7 protein is administered at a dose of about 1,500 μg/kg. In some aspects, an IL-7 protein is administered at a dose of about 1,520 μg/kg. In some aspects, an IL-7 protein is administered at a dose of about 1,540 μg/kg. In some aspects, an IL-7 protein is administered at a dose of about 1,560 μg/kg. In some aspects, an IL-7 protein is administered at a dose of about 1,580 μg/kg. In some aspects, an IL-7 protein is administered at a dose of about 1,600 μg/kg. In some aspects, an IL-7 protein is administered at a dose of about 1,620 μg/kg. In some aspects, an IL-7 protein is administered at a dose of about 1,640 μg/kg. In some aspects, an IL-7 protein is administered at a dose of about 1,660 μg/kg. In some aspects, an IL-7 protein is administered at a dose of about 1,680 μg/kg. In some aspects, an IL-7 protein is administered at a dose of about 1,700 μg/kg. In some aspects, an IL-7 protein is administered at a dose of about 1,720 μg/kg. In some aspects, an IL-7 protein is administered at a dose of about 1,740 μg/kg. In some aspects, an IL-7 protein is administered at a dose of about 1,760 μg/kg. In some aspects, an IL-7 protein is administered at a dose of about 1,780 μg/kg. In some aspects, an IL-7 protein is administered at a dose of about 1,800 μg/kg. In some aspects, an IL-7 protein is administered at a dose of about 1,820 μg/kg. In some aspects, an IL-7 protein is administered at a dose of about 1,840 μg/kg. In some aspects, an IL-7 protein is administered at a dose of about 1,860 μg/kg. In some aspects, an IL-7 protein is administered at a dose of about 1,880 μg/kg. In some aspects, an IL-7 protein is administered at a dose of about 1,900 μg/kg. In some aspects, an IL-7 protein is administered at a dose of about 1,920 μg/kg. In some aspects, an IL-7 protein is administered at a dose of about 1,940 μg/kg. In some aspects, an IL-7 protein is administered at a dose of about 1,960 μg/kg. In some aspects, an IL-7 protein is administered at a dose of about 1,980 μg/kg. In some aspects, an IL-7 protein is administered at a dose of about 2,000 μg/kg.

In some aspects, an IL-7 protein described herein is administered to the subject at a dose of about 0.01 nmol/kg, about 0.02 nmol/kg, about 0.03 nmol/kg, about 0.04 nmol/kg, about 0.05 nmol/kg, about 0.1 nmol/kg, about 0.2 nmol/kg, about 0.4 nmol/kg, about 0.6 nmol/kg, about 0.8 nmol/kg, about 1 nmol/kg, about 1.2 nmol/kg, about 1.4 nmol/kg, about 1.6 nmol/kg, about 1.8 nmol/kg, about 2 nmol/kg, about 2.2 nmol/kg, about 2.4 nmol/kg, about 2.6 nmol/kg, about 2.8 nmol/kg, about 3 nmol/kg, about 3.5 nmol/kg, about 4 nmol/kg, about 4.5 nmol/kg, about 5 nmol/kg, about 6 nmol/kg, about 7 nmol/kg, about 8 nmol/kg, about 9 nmol/kg, about 10 nmol/kg, about 11 nmol/kg, about 12 nmol/kg, about 13 nmol/kg, about 14 nmol/kg, about 15 nmol/kg, about 16 nmol/kg, about 17 nmol/kg, about 18 nmol/kg, about 19 nmol/kg, about 20 nmol/kg, about 22 nmol/kg, about 24 nmol/kg, about 26 nmol/kg, about 28 nmol/kg, about 30 nmol/kg, about 32 nmol/kg, about 34 nmol/kg, about 36 nmol/kg, about 38 nmol/kg, or about 40 nmol/kg.

In some aspects, an IL-7 protein can be administered at a flat dose. In some aspects, an IL-7 protein can be administered at a flat dose of about 0.25 mg to about 9 mg. In some aspects, an IL-7 protein can be administered at a flat dose of about 0.25 mg, about 1 mg, about 3 mg, about 6 mg, or about 9 mg.

In some aspects, a subject disclosed herein receives a single administration of an IL-7 protein at any of the doses described above. In some aspects, an IL-7 protein disclosed herein is administered to a subject at multiple doses (i.e., repeated administrations). In some aspects, an IL-7 protein is administered to the subject at least about two times, at least about three times, at least about four times, at least about five times, at least about six times, at least about seven times, at least about eight times, at least about nine times, or at least about ten times or more. In some aspects, an IL-7 protein is administered to the subject about two times. In some aspects, an IL-7 protein is administered to the subject about three times. In some aspects, an IL-7 protein is administered to the subject about four times. In some aspects, an IL-7 protein is administered to the subject about five times. In some aspects, an IL-7 protein is administered to the subject about six times. In some aspects, an IL-7 protein is administered to the subject about seven times. In some aspects, an IL-7 protein is administered to the subject about eight times. In some aspects, an IL-7 protein is administered to the subject about nine times. In some aspects, an IL-7 protein is administered to the subject about ten times or more.

Where a subject (e.g., afflicted with a tumor comprising an unmethylated MGMT promoter) receives multiple administration of an IL-7 protein, in some aspects, the IL-7 protein is administered at a dosing frequency of about once a week, about once in two weeks, about once in three weeks, about once in four weeks, about once in five weeks, about once in six weeks, about once in seven weeks, about once in eight weeks, about once in nine weeks, about once in 10 weeks, about once in 11 weeks, or about once in 12 weeks, e.g., at one of the doses described above. In some aspects, an IL-7 protein is administered at a dosing frequency of about once every 10 days, about once every 20 days, about once every 30 days, about once every 40 days, about once every 50 days, about once every 60 days, about once every 70 days, about once every 80 days, about once every 90 days, or about once every 100 days. In some aspects, the IL-7 protein is administered once in three weeks. In some aspects, the IL-7 protein is administered once a week. In some aspects, the IL-7 protein is administered once in two weeks. In some aspects, the IL-7 protein is administered once in four weeks. In some aspects, the IL-7 protein is administered once in six weeks. In some aspects, the IL-7 protein is administered once in eight weeks. In some aspects, the IL-7 protein is administered once in nine weeks. In some aspects, the IL-7 protein is administered once in 12 weeks. In some aspects, the IL-7 protein is administered once every 10 days. In some aspects, the IL-7 protein is administered once every 20 days. In some aspects, the IL-7 protein is administered once every 30 days. In some aspects, the IL-7 protein is administered once every 40 days. In some aspects, the IL-7 protein is administered once every 50 days. In some aspects, the IL-7 protein is administered once every 60 days. In some aspects, the IL-7 protein is administered once every 70 days. In some aspects, the IL-7 protein is administered once every 80 days. In some aspects, the IL-7 protein is administered once every 90 days. In some aspects, the IL-7 protein is administered once every 100 days.

As described herein, some aspects of the present disclosure comprises administering to a subject (e.g., suffering from a tumor with an unmethylated MGMT promoter) an IL-7 protein in combination with an additional therapeutic agent. In some aspects, the additional therapeutic agent comprises a chemotherapy. In some aspects, the chemotherapy comprises TMZ. Non-limiting examples of other additional therapeutic agents that can be used are provided elsewhere in the present disclosure.

In some aspects, a subject that can be treated with the present disclosure receives a single administration of the additional therapeutic agent. In some aspects, an additional therapeutic agent is administered to a subject at multiple doses (i.e., repeated administration). In some aspects, an additional therapeutic agent (e.g., chemotherapy, e.g., TMZ) is administered to the subject at least about two times, at least about three times, at least about four times, at least about five times, at least about six times, at least about seven times, at least about eight times, at least about nine times, or at least about ten times or more. In some aspects, an additional therapeutic agent (e.g., chemotherapy, e.g., TMZ) is administered to the subject about two times. In some aspects, an additional therapeutic agent (e.g., chemotherapy, e.g., TMZ) is administered to the subject about three times. In some aspects, an additional therapeutic agent (e.g., chemotherapy, e.g., TMZ) is administered to the subject about four times. In some aspects, an additional therapeutic agent (e.g., chemotherapy, e.g., TMZ) is administered to the subject about five times. In some aspects, an additional therapeutic agent (e.g., chemotherapy, e.g., TMZ) is administered to the subject about six times. In some aspects, an additional therapeutic agent (e.g., chemotherapy, e.g., TMZ) is administered to the subject about seven times. In some aspects, an additional therapeutic agent (e.g., chemotherapy, e.g., TMZ) is administered to the subject about eight times. In some aspects, an additional therapeutic agent (e.g., chemotherapy, e.g., TMZ) is administered to the subject about nine times. In some aspects, an additional therapeutic agent (e.g., chemotherapy, e.g., TMZ) is administered to the subject about ten times or more.

Where a subject (e.g., afflicted with a tumor comprising an unmethylated MGMT promoter) receives multiple administration of an additional therapeutic agent, in some aspects, the additional therapeutic agent is administered at a dosing frequency of about once a week, about once in two weeks, about once in three weeks, about once in four weeks, about once in five weeks, about once in six weeks, about once in seven weeks, about once in eight weeks, about once in nine weeks, about once in 10 weeks, about once in 11 weeks, or about once in 12 weeks, e.g., at one of the doses described above. In some aspects, the additional therapeutic agent is administered once in three weeks. In some aspects, the additional therapeutic agent is administered once a week. In some aspects, the additional therapeutic agent is administered once in two weeks. In some aspects, the additional therapeutic agent is administered once in four weeks. In some aspects, the additional therapeutic agent is administered once in six weeks. In some aspects, the additional therapeutic agent is administered once in eight weeks. In some aspects, the additional therapeutic agent is administered once in nine weeks. In some aspects, the additional therapeutic agent is administered once in 12 weeks.

In some aspects, a subject (e.g., afflicted with a tumor comprising an unmethylated MGMT promoter) receives multiple administration of an IL-7 protein and multiple administration of an additional therapeutic agent (e.g., chemotherapy, e.g., TMZ). In some aspects, the subject receives: (i) at least about two doses, at least about three doses, at least about four doses, or at least about five doses of the IL-7 protein, and (ii) at least about two doses, at least about three doses, at least about four doses, at least about five doses, at least about six doses, or at least about seven doses of the additional agent. In some aspects, the subject receives at least about four doses of the IL-7 protein and at least about six doses of the additional agent. In some aspects, the IL-7 protein is administered to the subject once about every 12 weeks and the additional agent is administered to the subject once about every four weeks. In some aspects, the subject receives about four doses of the IL-7 protein and about six does of the additional agent, wherein the IL-7 protein is administered to the subject once about every 12 weeks and the additional agent is administered to the subject once about every four weeks.

In some aspects, one or more doses of the additional agent (e.g., chemotherapy, e.g., TMZ) is administered to the subject over a course of about a single day, over a course of about two consecutive days, over a course of about three consecutive days, over a course of about four consecutive days, over a course of about five consecutive days, over a course of about six consecutive days, or over a course of about seven consecutive days. In some aspects, one or more doses of the additional agent is administered to a subject over a course of about a single day. In some aspects, one or more does of the additional agent is administered to the subject over a course of about two consecutive days. In some aspects, one or more does of the additional agent is administered to the subject over a course of about three consecutive days. In some aspects, one or more does of the additional agent is administered to the subject over a course of about four consecutive days. In some aspects, one or more does of the additional agent is administered to the subject over a course of about five consecutive days. In some aspects, one or more does of the additional agent is administered to the subject over a course of about six consecutive days. In some aspects, one or more does of the additional agent is administered to the subject over a course of about seven consecutive days. In some aspects, the additional therapeutic agent is administered to the subject on days 1-5 of a 28-day cycle.

### 5. IL-7 Proteins Useful for the Disclosure

Disclosed herein are IL-7 proteins that can be used to treat a tumor described herein (e.g., comprising an unmethylated MGMT promoter). In some aspects, IL-7 protein useful for the present uses can be wild-type IL-7 or modified IL-7 (i.e., not wild-type IL-7 protein) (e.g., IL-7 variant, IL-7 functional fragment, IL-7 derivative, or any combination thereof, e.g., fusion protein, chimeric protein, etc.) as long as the IL-7 protein contains one or more biological activities of IL-7, e.g., capable of binding to IL-7R, e.g., inducing early T-cell development, promoting T-cell homeostasis. See ElKassar and Gress. J Immunotoxicol. 2010 March; 7 (1): 1-7. In some aspects, an IL-7 protein of the present disclosure is not a wild-type IL-7 protein (i.e., comprises one or more modifications). Non-limiting examples of such modifications can include an oligopeptide and/or a half-life extending moiety. See WO 2016/200219, which is herein incorporated by reference in its entirety.

IL-7 binds to its receptor which is composed of the two chains IL-7Ra (CD127), shared with the thymic stromal lymphopoietin (TSLP) (Ziegler and Liu, 2006), and the common γ chain (CD132) for IL-2, IL-15, IL-9 and IL-21. Whereas γc is expressed by most hematopoietic cells, IL-7Rα is nearly exclusively expressed on lymphoid cells. After binding to its receptor, IL-7 signals through two different pathways: Jak-Stat (Janus kinase-Signal transducer and activator of transcription) and PI3K/Akt responsible for differentiation and survival, respectively. The absence of IL-7 signaling is responsible for a reduced thymic cellularity as observed in mice that have received an anti-IL-7 neutralizing monoclonal antibody (MAb); Grabstein et al., 1993), in IL-7−/−(von Freeden-Jeffry et al., 1995), IL-7Ra−/−(Peschon et al., 1994; Maki et al., 1996), γc−/−(Malissen et al., 1997), and Jak3−/− mice (Park et al., 1995). In the absence of IL-7 signaling, mice lack T-, B-, and NK-T cells. IL-7a−/−mice (Peschon et al., 1994) have a similar but more severe phenotype than IL-7−/−mice (von Freeden-Jeffry et al., 1995), possibly because TSLP signaling is also abrogated in IL-7a−/−mice. IL-7 is required for the development of γδ cells (Maki et al., 1996) and NK-T cells (Boesteanu et al., 1997).

In some aspects, an IL-7 protein useful for the present disclosure comprises an amino acid sequence as set forth in any one of SEQ ID NOs: 1 to 6 and 80 to 85. In some aspects, the IL-7 protein comprises an amino acid sequence having a sequence identity of about 70%, about 75%, about 80%, about 85%, about 90%, about 91%, about 92%, about 93%, about 94%, about 95%, about 96%, about 97%, about 98%, or about 99% or higher, to a sequence of SEQ ID NOS: 1 to 6. In some aspects, an IL-7 protein that can be used with the present disclosure comprises the amino acid sequence set forth in SEQ ID NO: 80 (i.e., the amino acid sequence set forth in SEQ ID NO: 1 but without the signal peptide). In some aspects, an IL-7 protein that can be used with the present disclosure comprises the amino acid sequence set forth in SEQ ID NO: 81 (i.e., the amino acid sequence set forth in SEQ ID NO: 2 but without the signal peptide). In some aspects, an IL-7 protein that can be used with the present disclosure comprises the amino acid sequence set forth in SEQ ID NO: 82 (i.e., the amino acid sequence set forth in SEQ ID NO: 3 but without the signal peptide). In some aspects, an IL-7 protein that can be used with the present disclosure comprises the amino acid sequence set forth in SEQ ID NO: 83 (i.e., the amino acid sequence set forth in SEQ ID NO: 4 but without the signal peptide). In some aspects, an IL-7 protein that can be used with the present disclosure comprises the amino acid sequence set forth in SEQ ID NO: 84 (i.e., the amino acid sequence set forth in SEQ ID NO: 5 but without the signal peptide). In some aspects, an IL-7 protein that can be used with the present disclosure comprises the amino acid sequence set forth in SEQ ID NO: 85 (i.e., the amino acid sequence set forth in SEQ ID NO: 6 but without the signal peptide).

In some aspects, the IL-7 protein includes a modified IL-7 or a fragment thereof, wherein the modified IL-7 or the fragment retains one or more biological activities of wild-type IL-7. In some aspects, the IL-7 protein can be derived from humans, rats, mice, monkeys, cows, or sheep.

In some aspects, the human IL-7 can have an amino acid sequence represented by SEQ ID NO: 1 (Genbank Accession No. P13232); the rat IL-7 can have an amino acid sequence represented by SEQ ID NO: 2 (Genbank Accession No. P56478); the mouse IL-7 can have an amino acid sequence represented by SEQ ID NO: 3 (Genbank Accession No. P10168); the monkey IL-7 can have an amino acid sequence represented by SEQ ID NO: 4 (Genbank Accession No. NP 001279008); the cow IL-7 can have an amino acid sequence represented by SEQ ID NO: 5 (Genbank Accession No. P26895), and the sheep IL-7 can have an amino acid sequence represented by SEQ ID NO: 6 (Genbank Accession No. Q28540).

In some aspects, an IL-7 protein useful for the present disclosure comprises an IL-7 fusion protein. In some aspects, an IL-7 fusion protein comprises (i) an oligopeptide and (i) an IL-7 or a variant thereof. In some aspects, the oligopeptide is linked to the N-terminal region of the IL-7 or a variant thereof.

In some aspects, an oligopeptide disclosed herein consists of 1 to 10 amino acids. In some aspects, an oligopeptide consists of at least 1, at least 2, at least 3, at least 4, at least 5, at least 6, at least 7, at least 8, at least 9, or 10 amino acids. In some aspects, one or more amino acids of an oligopeptide are selected from the group consisting of methionine, glycine, and combinations thereof. In some aspects, an oligopeptide is selected from the group consisting of methionine, glycine, methionine-methionine, glycine-glycine, methionine-glycine, glycine-methionine, methionine-methionine-methionine, methionine-methionine-glycine, methionine-glycine-methionine, glycine-methionine-methionine, methionine-glycine-glycine, glycine-methionine-glycine, glycine-glycine-methionine, and glycine-glycine-glycine. In some aspects, an oligopeptide is methionine-glycine-methionine.

In some aspects, an IL-7 fusion protein comprises (i) an IL-7 or a variant thereof, and (ii) a half-life extending moiety. In some aspects, a half-life extending moiety extends the half-life of the IL-7 or variant thereof. In some aspects, a half-life extending moiety is linked to the C-terminal region of an IL-7 or a variant thereof.

In some aspects, an IL-7 fusion protein comprises (i) IL-7 (a first domain), (ii) a second domain that includes an amino acid sequence having 1 to 10 amino acid residues consisting of methionine, glycine, or a combination thereof, e.g., MGM, and (iii) a third domain comprising a half-life extending moiety. In some aspects, the half-life extending moiety can be linked to the N-terminal or the C-terminal of the first domain or the second domain. Additionally, the IL-7 including the first domain and the second domain can be linked to both terminals of the third domain.

Non-limiting examples of half-life extending moieties include: Fc, albumin, an albumin-binding polypeptide, Pro/Ala/Ser (PAS), a C-terminal peptide (CTP) of the β subunit of human chorionic gonadotropin, polyethylene glycol (PEG), long unstructured hydrophilic sequences of amino acids (XTEN), hydroxyethyl starch (HES), an albumin-binding small molecule, and combinations thereof.

In some aspects, a half-life extending moiety is Fc. In some aspects, Fc is from a modified immunoglobulin in which the antibody-dependent cellular cytotoxicity (ADCC) or complement-dependent cytotoxicity (CDC) weakened due to the modification in the binding affinity with the Fc receptor and/or a complement. In some aspects, the modified immunoglobulin can be selected from the group consisting of IgG1, IgG2, IgG3, IgG4, IgA1, IgA2, IgD, IgE, and a combination thereof. In some aspects, an Fc is a hybrid Fc (“hFc” or “hyFc”), comprising a hinge region, a CH2 domain, and a CH3 domain. In some aspects, a hinge region of a hybrid Fc disclosed herein comprises a human IgD hinge region. In some aspects, a CH2 domain of a hybrid Fc comprises a part of human IgD CH2 domain and a part of human IgG4 CH2 domain. In some aspects, a CH3 domain of a hybrid Fc comprises a part of human IgG4 CH3 domain. Accordingly, in some aspects, a hybrid Fc disclosed herein comprises a hinge region, a CH2 domain, and a CH3 domain, wherein the hinge region comprises a human IgD hinge region, wherein the CH2 domain comprises a part of human IgD CH2 domain and a part of human IgG4 CH2 domain, and wherein the CH3 domain comprises a part of human IgG4 CH3 domain.

In some aspects, an Fc disclosed herein can be an Fc variant. As used herein, the term “Fc variant” refers to an Fc which was prepared by substituting a part of the amino acids among the Fc region or by combining the Fc regions of different kinds. The Fc region variant can prevent from being cut off at the hinge region. Specifically, in some aspects, a Fc variant comprises modifications at the 144th amino acid and/or 145th amino acid of SEQ ID NO: 9. In some aspects, the 144th amino acid (K) and/or the 145th amino acid (K) is substituted with G or S.

In some aspects, an Fc or an Fc variant disclosed herein can be represented by the following formula: N′-(Z1) p-Y-Z2-Z3-Z4-C, wherein:
N′ comprises the N-terminus;
Z1 comprises an amino acid sequence having 5 to 9 consecutive amino acid residues from the amino acid residue at position 98 toward the N-terminal, among the amino acid residues at positions from 90 to 98 of SEQ ID NO: 7;
Y comprises an amino acid sequence having 5 to 64 consecutive amino acid residues from the amino acid residue at position 162 toward the N-terminal, among the amino acid residues at positions from 99 to 162 of SEQ ID NO: 7;
Z2 comprises an amino acid sequence having 4 to 37 consecutive amino acid residues from the amino acid residue at position 163 toward the C-terminal, among the amino acid residues at positions from 163 to 199 of SEQ ID NO: 7;
Z3 comprises an amino acid sequence having 71 to 106 consecutive amino acid residues from the amino acid residue at position 220 toward the N-terminal, among the amino acid residues at positions from 115 to 220 of SEQ ID NO: 8; and
Z4 comprises an amino acid sequence having 80 to 107 consecutive amino acid residues from the amino acid residue at position 221 toward the C-terminal, among the amino acid residues at positions from 221 to 327 of SEQ ID NO: 8.

In some aspects, a Fc region disclosed herein can include the amino acid sequence of SEQ ID NO: 9 (hyFc), SEQ ID NO: 10 (hyFcM1), SEQ ID NO: 11 (hyFcM2), SEQ ID NO: 12 (hyFcM3), or SEQ ID NO: 13 (hyFcM4). In some aspects, the Fc region can include the amino acid sequence of SEQ ID NO: 14 (a non-lytic mouse Fc). Non-limiting examples of Fc sequences are provided in Table 2 (below).

Other non-limiting examples of Fc regions that can be used with the present disclosure are described in U.S. Pat. No. 7,867,491, which is herein incorporated by reference in its entirety.

In some aspects, an IL-7 fusion protein disclosed herein comprises both an oligopeptide and a half-life extending moiety.

In some aspects, an IL-7 protein can be fused to albumin, a variant, or a fragment thereof. Examples of the IL-7-albumin fusion protein can be found at International Application Publication No. WO 2011/124718 A1, which is incorporated herein by reference in its entirety. In some aspects, an IL-7 protein is fused to a pre-pro-B cell Growth Stimulating Factor (PPBSF), optionally by a flexible linker. See US 2002/0058791A1, which is incorporated herein by reference in its entirety. In some aspects, an IL-7 protein useful for the disclosure is an IL-7 conformer that has a particular three dimensional structure. See US 2005/0249701 A1, which is incorporated herein by reference in its entirety. In some aspects, an IL-7 protein can be fused to an Ig chain, wherein amino acid residues 70 and 91 in the IL-7 protein are glycosylated the amino acid residue 116 in the IL-7 protein is non-glycosylated. See U.S. Pat. No. 7,323,549 B2, which is incorporated herein by reference in its entirety. In some aspects, an IL-7 protein that does not contain potential T-cell epitopes (thereby to reduce anti-IL-7 T-cell responses) can also be used for the present disclosure. See US 2006/0141581 A1, which is incorporated herein by reference in its entirety. In some aspects, an IL-7 protein that has one or more amino acid residue mutations in carboxy-terminal helix D region can be used for the present disclosure. The IL-7 mutant can act as IL-7R partial agonist despite lower binding affinity for the receptor. See US 2005/0054054A1, which is incorporated herein by reference in its entirety. Any IL-7 proteins described in the above listed patents or publications are incorporated herein by reference in their entireties.

In addition, non-limiting examples of additional IL-7 proteins useful for the present disclosure are described in U.S. Pat. Nos. 7,708,985, 8,034,327, 8,153,114, 7,589,179, 7,323,549, 7,960,514, 8,338,575, 7,118,754, 7,488,482, 7,670,607, 6,730,512, WO0017362, GB2434578A, WO 2010/020766 A2, WO91/01143, Beq et al., Blood, vol. 114 (4), 816, 23 Jul. 2009, Kang et al., J. Virol. Doi: 10.1128/JVI.02768-15, Martin et al., Blood, vol. 121 (22), 4484 May 30, 2013, McBride et al., Acta Oncologica, 34:3, 447-451, Jul. 8, 2009, and Xu et al., Cancer Science, 109:279-288, 2018, which are incorporated herein by reference in their entireties.

In some aspects, an oligopeptide disclosed herein is directly linked to the N-terminal region of IL-7 or a variant thereof. In some aspects, an oligopeptide is linked to the N-terminal region via a linker. In some aspects, a half-life extending moiety disclosed herein is directly linked to the C-terminal region of IL-7 or a variant thereof. In some aspects, a half-life extending moiety is linked to the C-terminal region via a linker. In some aspects, a linker is a peptide linker. In some aspects, a peptide linker comprises a peptide of 10 to 20 amino acid residues consisting of Gly and Ser residues. In some aspects, a linker is an albumin linker. In some aspects, a linker is a chemical bond. In some aspects, a chemical bond comprises a disulfide bond, a diamine bond, a sulfide-amine bond, a carboxy-amine bond, an ester bond, a covalent bond, or combinations thereof. When the linker is a peptide linker, in some aspects, the connection can occur in any linking region. They can be coupled using a crosslinking agent known in the art. In some aspects, examples of the crosslinking agent can include N-hydroxy succinimide esters such as 1,1-bis(diazoacetyl)-2-phenylethane, glutaraldehyde, and 4-azidosalicylic acid; imido esters including disuccinimidyl esters such as 3,3′-dithiobis (succinimidyl propionate), and bifunctional maleimides such as bis-Nmaleimido-1,8-octane, but is not limited thereto.

In some aspects, an IL-7 (or variant thereof) portion of IL-7 fusion protein disclosed herein comprises an amino sequence that is at least 70%, at least 75%, at least 80%, at least 85%, at least 90%, at least 91%, at least 92%, at least 93%, at least 94%, at least 95%, at least 96%, at least 97%, at least 98%, at least 98%, or at least 99% identical to an amino acid sequence set forth in SEQ ID NOs: 15-20. In some aspects, an IL-7 (or variant thereof) portion of IL-7 fusion protein disclosed herein comprises the amino acid sequence set forth in SEQ ID NOs: 15-20.

In some aspects, an IL-7 fusion protein comprises: a first domain including a polypeptide having the activity of IL-7 or a similar activity thereof; a second domain comprising an amino acid sequence having 1 to 10 amino acid residues consisting of methionine, glycine, or a combination thereof; and a third domain, which is an Fc region of modified immunoglobulin, coupled to the C-terminal of the first domain.

In some aspects, an IL-7 fusion protein that can be used with the present methods comprises an amino sequence that is at least 70%, at least 75%, at least 80%, at least 85%, at least 90%, at least 91%, at least 92%, at least 93%, at least 94%, at least 95%, at least 96%, at least 97%, at least 98%, at least 98%, or at least 99% identical to an amino acid sequence set forth in SEQ ID NOs: 21-25. In some aspects, an IL-7 fusion protein of the present disclosure comprises the amino acid sequence set forth in SEQ ID NOs: 21-25. In some aspects, an IL-7 fusion protein disclosed herein comprises the amino acid sequence set forth in SEQ ID NOs: 26 and 27. Exemplary IL-7 protein/fusion protein sequences are provided in Table 2 (below).

Unless indicated otherwise, an IL-7 protein described (including fusion proteins) herein can be administered to a subject (e.g., in combination with an additional therapeutic agent, e.g., chemotherapy, e.g., TMZ) using any suitable routes of administration. In some aspects, the IL-7 protein is administered to the subject intravenously, parenthetically, intramuscularly, subcutaneously, ophthalmic, intraperitoneally, intradermally, intraorbitally, intracerebrally, intracranially, intraspinally, intraventricular, intrathecally, intracistemally, intracapsularly, or intratumorally.

### III. Nucleic Acids, Vectors, Host Cells

Some aspects described herein pertain to one or more nucleic acid molecules that encode a therapeutic agent described herein (e.g., an IL-7 protein and/or an additional therapeutic agent). The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. See, F. Ausubel, et al., ed. (1987) Current Protocols in Molecular Biology, Greene Publishing and Wiley Interscience, New York. A nucleic acid described herein can be, for example, DNA or RNA and can or cannot contain intronic sequences. In some aspects, the nucleic acid is a cDNA molecule. Nucleic acids described herein can be obtained using standard molecular biology techniques known in the art.

Some nucleic acid molecules disclosed herein are those encoding an IL-7 protein (e.g., disclosed herein) that can be used alone or in combination with an additional therapeutic agent, e.g., to treat a tumor with an unmethylated MGMT tumor. Exemplary nucleic acid sequences encoding an IL-7 protein disclosed herein are set forth in SEQ ID NOs: 29-39 (see Table 2, below).

In some aspects, the present disclosure provides a vector comprising an isolated nucleic acid molecule encoding a therapeutic agent disclosed herein (e.g., an IL-7 protein and/or an additional therapeutic agent). In some aspects, a vector can be used for gene therapy.

When used as a gene therapy (e.g., in humans), a nucleic acid encoding a therapeutic agent disclosed herein (e.g., an IL-7 protein and/or an additional therapeutic agent) can be administered at a dosage in the range of 0.1 mg to 200 mg. In some aspects, the dosage is in the range of 0.6 mg to 100 mg. In some aspects, the dosage is in the range of 1.2 mg to 50 mg.

Suitable vectors for the disclosure include expression vectors, viral vectors, and plasmid vectors. In some aspects, the vector is a viral vector.

As used herein, an expression vector refers to any nucleic acid construct which contains the necessary elements for the transcription and translation of an inserted coding sequence, or in the case of an RNA viral vector, the necessary elements for replication and translation, when introduced into an appropriate host cell. Expression vectors can include plasmids, phagemids, viruses, and derivatives thereof.

As used herein, viral vectors include, but are not limited to, nucleic acid sequences from the following viruses: retrovirus, such as Moloney murine leukemia virus, Harvey murine sarcoma virus, murine mammary tumor virus, and Rous sarcoma virus; lentivirus; adenovirus; adeno-associated virus; SV40-type viruses; polyomaviruses; Epstein-Barr viruses; papilloma viruses; herpes virus; vaccinia virus; polio virus; and RNA virus such as a retrovirus. One can readily employ other vectors well-known in the art. Some viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA.

In some aspects, a vector is derived from an adeno-associated virus. In some aspects, a vector is derived from a lentivirus. Examples of the lentiviral vectors are disclosed in WO9931251, WO9712622, WO9817815, WO9817816, and WO9818934, each which is incorporated herein by reference in its entirety.

Other vectors include plasmid vectors. Plasmid vectors have been extensively described in the art and are well-known to those of skill in the art. See, e.g., Sambrook et al., Molecular Cloning: A Laboratory Manual, Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operably encoded within the plasmid. Some commonly used plasmids available from commercial suppliers include pBR322, pUC18, pUC19, various pcDNA plasmids, pRC/CMV, various pCMV plasmids, pSV40, and pBlueScript. Additional examples of specific plasmids include pcDNA3.1, catalog number V79020; pcDNA3.1/hygro, catalog number V87020; pcDNA4/myc-His, catalog number V86320; and pBudCE4.1, catalog number V53220, all from Invitrogen (Carlsbad, CA.). Other plasmids are well-known to those of ordinary skill in the art. Additionally, plasmids can be custom designed using standard molecular biology techniques to remove and/or add specific fragments of DNA.

Also encompassed by the present disclosure is a method for making a therapeutic agent disclosed herein (e.g., an IL-7 protein). In some aspects, such a method can comprise expressing the therapeutic agent (e.g., an IL-7 protein) in a cell comprising a nucleic acid molecule encoding the therapeutic agent, e.g., SEQ ID NOs: 29-39. Additional details regarding the method for making an IL-7 protein disclosed herein are provided, e.g., in U.S. Pat. No. 11,041,007, which is herein incorporated by reference in its entirety. Host cells comprising these nucleotide sequences are encompassed herein. Non-limiting examples of host cell that can be used include immortal hybridoma cell, NS/0 myeloma cell, 293 cell, Chinese hamster ovary (CHO) cell, HeLa cell, human amniotic fluid-derived cell (CapT cell), COS cell, or combinations thereof.

### IV. Pharmaceutical Compositions

Further provided herein are compositions comprising one or more therapeutic agents (e.g., an IL-7 protein and/or an additional therapeutic agent) having the desired degree of purity in a physiologically acceptable carrier, excipient or stabilizer (Remington's Pharmaceutical Sciences (1990) Mack Publishing Co., Easton, PA).

Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

In some aspects, a composition disclosed herein comprises one or more additional components selected from: a bulking agent, stabilizing agent, surfactant, buffering agent, or combinations thereof.

Buffering agents useful for the current disclosure can be a weak acid or base used to maintain the acidity (pH) of a solution near a chosen value after the addition of another acid or base. Suitable buffering agents can maximize the stability of the pharmaceutical compositions by maintaining pH control of the composition. Suitable buffering agents can also ensure physiological compatibility or optimize solubility. Rheology, viscosity and other properties can also dependent on the pH of the composition. Common buffering agents include, but are not limited to, a Tris buffer, a Tris-Cl buffer, a histidine buffer, a TAE buffer, a HEPES buffer, a TBE buffer, a sodium phosphate buffer, a MES buffer, an ammonium sulfate buffer, a potassium phosphate buffer, a potassium thiocyanate buffer, a succinate buffer, a tartrate buffer, a DIPSO buffer, a HEPPSO buffer, a POPSO buffer, a PIPES buffer, a PBS buffer, a MOPS buffer, an acetate buffer, a phosphate buffer, a cacodylate buffer, a glycine buffer, a sulfate buffer, an imidazole buffer, a guanidine hydrochloride buffer, a phosphate-citrate buffer, a borate buffer, a malonate buffer, a 3-picoline buffer, a 2-picoline buffer, a 4-picoline buffer, a 3,5-lutidine buffer, a 3,4-lutidine buffer, a 2,4-lutidine buffer, a Aces, a diethylmalonate buffer, a N-methylimidazole buffer, a 1,2-dimethylimidazole buffer, a TAPS buffer, a bis-Tris buffer, a L-arginine buffer, a lactate buffer, a glycolate buffer, or combinations thereof.

In some aspects, a composition disclosed herein further comprises a bulking agent. Bulking agents can be added to a pharmaceutical product in order to add volume and mass to the product, thereby facilitating precise metering and handling thereof. Bulking agents that can be used with the present disclosure include, but are not limited to, sodium chloride (NaCl), mannitol, glycine, alanine, or combinations thereof.

In some aspects, a composition disclosed herein can also comprise a stabilizing agent. Non-limiting examples of stabilizing agents that can be used with the present disclosure include: sucrose, trehalose, raffinose, arginine, or combinations thereof.

In some aspects, a composition disclosed herein comprises a surfactant. In some aspects, the surfactant can be selected from the following: alkyl ethoxylate, nonylphenol ethoxylate, amine ethoxylate, polyethylene oxide, polypropylene oxide, fatty alcohols such as cetyl alcohol or oleyl alcohol, cocamide MEA, cocamide DEA, polysorbates, dodecyl dimethylamine oxide, or combinations thereof. In some aspects, the surfactant is polysorbate 20 or polysorbate 80.

In some aspects, a composition comprising an IL-7 protein can be formulated using the same formulation of an additional therapeutic agent (e.g., which is to be used in combination with the IL-7 protein). In some aspects, an IL-7 protein and an additional therapeutic agent are formulated using different formulations.

A pharmaceutical composition can be formulated for any route of administration to a subject. Specific examples of routes of administration include intramuscularly, subcutaneously, ophthalmic, intravenously, intraperitoneally, intradermally, intraorbitally, intracerebrally, intracranially, intraspinally, intraventricular, intrathecally, intracistemally, intracapsularly, or intratumorally. Parenteral administration, characterized by either subcutaneous, intramuscular or intravenous injection, is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Topical mixtures comprising an antibody are prepared as described for the local and systemic administration. The resulting mixture can be a solution, suspension, emulsions or the like and can be formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

An antibody or antigen-binding portion thereof described herein can be formulated as an aerosol for topical application, such as by inhalation (see, e.g., U.S. Pat. Nos. 4,044,126, 4,414,209 and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflations, alone or in combination with an inert carrier such as lactose. In such a case, the particles of the formulation will, in one aspect, have diameters of less than 50 microns, in one aspect less than 10 microns.

A therapeutic agent disclosed herein can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the antibody alone or in combination with other pharmaceutically acceptable excipients can also be administered.

Transdermal patches, including iontophoretic and electrophoretic devices, are well known to those of skill in the art, and can be used to administer an antibody. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957, each of which is herein incorporated by reference in its entirety.

In some aspects, a pharmaceutical composition comprising a therapeutic agent described herein is a lyophilized powder, which can be reconstituted for administration as solutions, emulsions and other mixtures. It can also be reconstituted and formulated as solids or gels. The lyophilized powder is prepared by dissolving an antibody or antigen-binding portion thereof described herein, or a pharmaceutically acceptable derivative thereof, in a suitable solvent. In some aspects, the lyophilized powder is sterile. The solvent can contain an excipient which improves the stability or other pharmacological component of the powder or reconstituted solution, prepared from the powder. Excipients that can be used include, but are not limited to, dextrose, sorbitol, fructose, corn syrup, xylitol, glycerin, glucose, sucrose or other suitable agent. The solvent can also contain a buffer, such as citrate, sodium or potassium phosphate or other such buffer known to those of skill in the art at, in one aspect, about neutral pH. Subsequent sterile filtration of the solution followed by lyophilization under standard conditions known to those of skill in the art provides the desired formulation. In some aspects, the resulting solution can be apportioned into vials for lyophilization. Each vial can contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

Compositions provided herein can also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

The compositions to be used for in vivo administration can be sterile. This is readily accomplished by filtration through, e.g., sterile filtration membranes.

The following examples are merely illustrative and should not be construed as limiting the scope of this disclosure in any way as many variations and equivalents will become apparent to those skilled in the art upon reading the present disclosure.

### Examples

### Example 1: Effect of IL-7 Protein Administration on Treating Tumors with Unmethylated MGMT Promoter

To demonstrate the effect anti-cancer treatments provided herein in treating tumors with unmethylated MGMT promoter, a Phase I clinical study was performed in patients with high-grade glioma, including GBM and anaplastic astrocytoma. A key inclusion criteria was an absolute lymphocyte count≥600 cells/mm³. Additional inclusion and exclusion criteria are described elsewhere in the present disclosure. In total, 19 patients were enrolled: median age 58.0 years [25-78], median baseline ALC 1000 cells/mm³ [400-2,000], median baseline dexamethasone use 0 mg/day [0-12]. 63% of the enrolled patients were MGMT promoter unmethylated (“unmethylated GBM”).

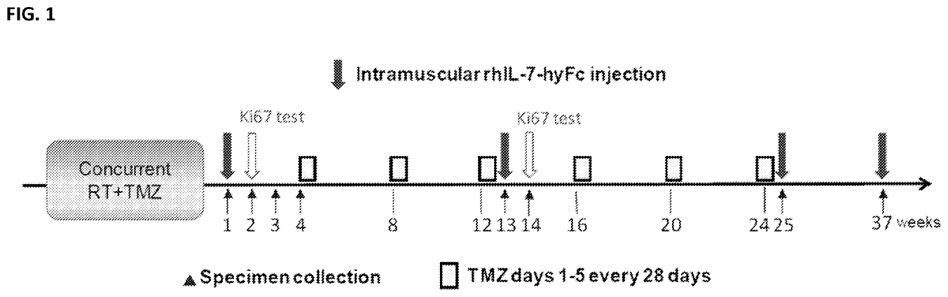

FIG. 1 provides the overall experimental design. As shown, an initial dose of an IL-7 protein described herein (long-acting) was administered to the patients within 2 weeks after completion of the concurrent radiotherapy (RT) and TMZ treatment (i.e., week 1). Additional doses of the IL-7 protein were administered every 12 weeks (i.e., at weeks 13, 25, and 37) for up to 4 total doses. The IL-7 protein was administered to the patients in escalating doses (60 μg/kg, 120 μg/kg, 240 μg/kg, 360 μg/kg, 540 μg/kg, 720 μg/kg, and 960 μg/kg) to determine the maximum tolerated dose (MTD). TMZ adjuvant was administered to the patients every 28 days over a course of five consecutive days for a total of six doses (i.e., at weeks 4, 8, 12, 16, 20, and 24).

Generally, the administered IL-7 protein was well-tolerated with grade 1/2 injection site reactions (42%) the most common treatment-related adverse event (TRAE). The MTD was 720 μg/kg due to two dose-limiting toxicities at 960 mcg/kg (a grade 3 elevated alanine aminotransferase and a grade 3 back pain). Dose-dependent increases in ALC were observed at 4 weeks (1.3-4.1× from baseline), persisting up to 12 weeks. As shown in Table 1, the median progression-free survival (mPFS) and median overall survival (mOS) for patients with methylated GBM were 19.1 months (95% CI: 15.1-NA) and 22 months (95% CI: 20.7-NA), respectively. For patients with unmethylated GBM, mPFS and mOS were 11.2 months (95% CI: 5.4-22.4) and 16 months (95% CI: 12.3-24.3), respectively. It has previously been shown that in patients with unmethylated GBM treated with radiotherapy with concurrent and adjuvant TMZ had mPFS and mOS of 4.99 months (95% CI: 4.25-5.72) and 14.11 months (95% CI: 13.18-15.04), respectively. Alnahhas et al., Neurooncol Adv 2 (1): vdaa082 (January-December 2020).

TABLE 1
Unmethylated

| | MGMT | MGMT | MGMT Unmethylated |
|---|---|---|---|
| HGG | Methylated | Unmethylated | (Alnahhas et al.) |
| mPFS | 19.1 months | 11.2 months | 4.99 months |
| mOS | 22 months | 16 months | 14.11 months |

The above results demonstrate the therapeutic potential of the IL-7 proteins described herein. In particular, the above results show that combining SOC with an IL-7 protein (such as that described herein) can greatly improve anti-tumor responses, resulting in improved survival.

### Example 2: Effect of IL-7 Protein Administration on Absolute Lymphocyte Count

To further demonstrate the therapeutic effects of anti-cancer treatments provided herein (e.g., IL-7 protein described herein alone or in combination with a TMZ adjuvant; see, e.g., Example 1), a randomized Phase II clinical study was performed in patients with high-grade glioma, including GBM and anaplastic astrocytoma. The primary objective of the study was to assess whether the administration of a IL-7 protein described herein (long-acting) results in increased absolute lymphocyte count (ALC) as compared to the placebo control. The effect on ALC was assessed at four weeks post IL-7 protein administration. The general study design was similar to that shown in FIG. 1. Some of the patients received a steroid treatment (e.g., to treat brain edema) prior to an anti-cancer treatment described herein (long-acting IL-7+TMZ). Accordingly, the different treatment groups were as follows: (a) placebo+TMZ adjuvant alone (i.e., no steroid), (b) steroid administration followed by placebo in combination with TMZ adjuvant, (c) long-acting IL-7 protein+TMZ alone (i.e., no steroid), or (d) steroid administration followed by long-acting IL-7 protein+TMZ.

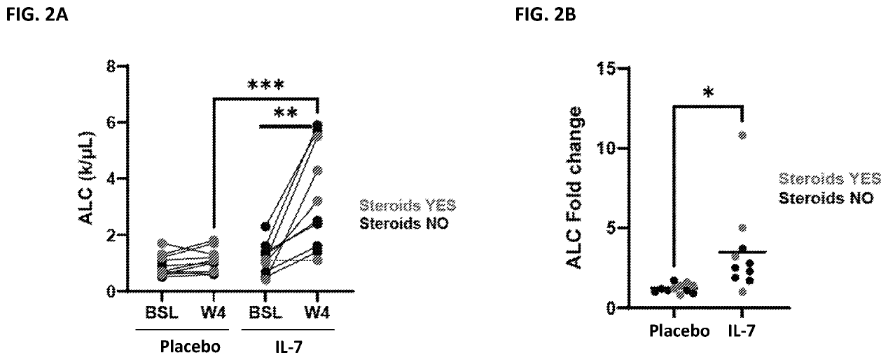

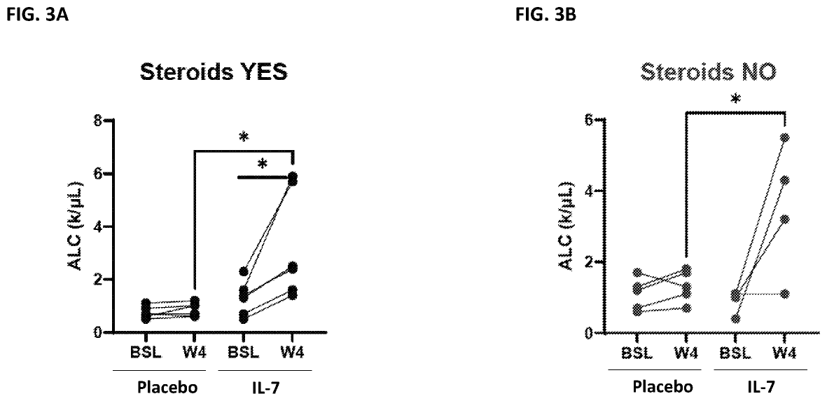

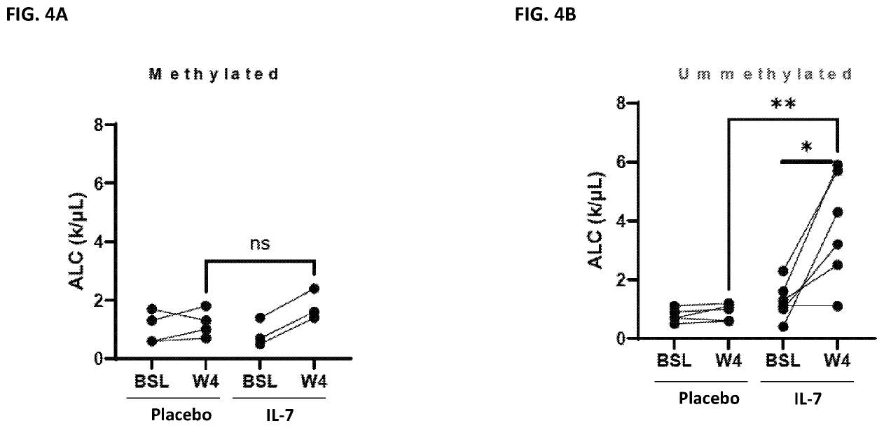

As shown in FIGS. 2A and 2B, in the placebo control group, there were no noticeable differences in ALC between baseline and at four weeks. In contrast, significant increase in ALC was observed in patients after IL-7 protein administration as compared to baseline. And, as shown in FIGS. 3A and 3B, the positive effect on ALC was observed regardless of whether the IL-7 protein was administered after or without steroid. The steroid is known to cause lymphopenia. Such data show that the therapeutic effects of the IL-7 protein administration is observed even under lymphopenic conditions (e.g., such as that induced by the steroid administration). Additionally, the greatest effect on ALC was observed in patients that were MGMT promoter unmethylated (see FIGS. 4A and 4B).

The above results further confirm the therapeutic efficacy of IL-7 proteins described herein and suggest that, such proteins (e.g., in combination with SOC) can be useful in treating tumors with an unmethylated O⁶-methylguanine-DNA methyltransferase (MGMT) promoter.

Table 2 (below) provides exemplary sequences relevant for the present application.

TABLE 2
Exemplary Sequences

| | |
|---|---|
| Human IL-7 Protein | MFHVSFRYIFGLPPLILVLLPVASSDCDIEGKDGKQYESVLMVSIDQLLDSMK |
| (SEQ ID NO: 1) | EIGSNCLNNEFNFFKRHICDANKEGMFLFRAARKLRQFLKMNSTGDFDLHLLK |
| (signal peptide | VSEGTTILLNCTGQVKGRKPAALGEAQPTKSLEENKSLKEQKKLNDLCFLKRL |
| bolded) | LQEIKTCWNKILMGTKEH |
| Rat IL-7 Protein | MFHVSFRYIFGIPPLILVLLPVTSSDCHIKDKDGKAFGSVLMISINQLDKMTG |
| (SEQ ID NO: 2) | TDSDCPNNEPNFFKKHLCDDTKEAAFLNRAARKLRQFLKMNISEEFNDHLLRV |
| (signal peptide | SDGTQTLVNCTSKEEKTIKEQKKNDPCFLKRLLREIKTCWNKILKGSI |
| bolded) | |
| Mouse IL-7 Protein | MFHVSFRYIFGIPPLILVLLPVTSSECHIKDKEGKAYESVLMISIDELDKMTG |
| (SEQ ID NO: 3) | TDSNCPNNEPNFFRKHVCDDTKEAAFLNRAARKLKQFLKMNISEEFNVHLLTV |
| (signal peptide | SQGTQTLVNCTSKEEKNVKEQKKNDACFLKRLLREIKTCWNKILKGSI |
| bolded) | |
| Monkey IL-7 Protein | MFHVSFRYIFGLPPLILVLLPVASSDCDIEGKDGKQYESVLMVSIDQLLDSMK |
| (SEQ ID NO: 4) | EIGSNCLNNEFNFFKRHLCDDNKEGMFLFRAARKLKQFLKMNSTGDFDLHLLK |
| (signal peptide | VSEGTTILLNCTGKVKGRKPAALGEPQPTKSLEENKSLKEQKKLNDSCFLKRL |
| bolded) | LQKIKTCWNKILMGTKEH |
| Cow IL-7 Protein | MFHVSFRYIFGIPPLILVLLPVASSDCDISGKDGGAYQNVLMVNIDDLDNMIN |
| (SEQ ID NO: 5) | FDSNCLNNEPNFFKKHSCDDNKEASFLNRASRKLRQFLKMNISDDFKLHLSTV |
| (signal peptide | SQGTLTLLNCTSKGKGRKPPSLSEAQPTKNLEENKSSKEQKKQNDLCFLKILL |
| bolded) | QKIKTCWNKILRGIKEH |
| Sheep IL-7 Protein | MFHVSFRYIFGIPPLILVLLPVASSDCDFSGKDGGAYQNVLMVSIDDLDNMIN |
| (SEQ ID NO: 6) | FDSNCLNNEPNFFKKHSCDDNKEASFLNRAARKLKQFLKMNISDDFKLHLSTV |
| (signal peptide | SQGTLTLLNCTSKGKGRKPPSLGEAQPTKNLEENKSLKEQRKQNDLCFLKILL |
| bolded) | QKIKTCWNKILRGITEH |
| Human IL-7 Protein | DCDIEGKDGKQYESVLMVSIDQLLDSMKEIGSNCLNNEFNFFKRHICDANKEG |
| (SEQ ID NO: 80) | MFLFRAARKLRQFLKMNSTGDFDLHLLKVSEGTTILLNCTGQVKGRKPAALGE |
| (without signal | AQPTKSLEENKSLKEQKKLNDLCFLKRLLQEIKTCWNKILMGTKEH |
| peptide) | |
| Rat IL-7 Protein | DCHIKDKDGKAFGSVLMISINQLDKMTGTDSDCPNNEPNFFKKHLCDDTKEAA |
| (SEQ ID NO: 81) | FLNRAARKLRQFLKMNISEEFNDHLLRVSDGTQTLVNCTSKEEKTIKEQKKND |
| (without signal | PCFLKRLLREIKTCWNKILKGSI |
| peptide) | |
| Mouse IL-7 Protein | ECHIKDKEGKAYESVLMISIDELDKMTGTDSNCPNNEPNFFRKHVCDDTKEAA |
| (SEQ ID NO: 82) | FLNRAARKLKQFLKMNISEEFNVHLLTVSQGTQTLVNCTSKEEKNVKEQKKND |
| (without signal | ACFLKRLLREIKTCWNKILKGSI |
| peptide) | |
| Monkey IL-7 Protein | DCDIEGKDGKQYESVLMVSIDQLLDSMKEIGSNCLNNEFNFFKRHLCDDNKEG |
| (SEQ ID NO: 83) | MFLFRAARKLKQFLKMNSTGDFDLHLLKVSEGTTILLNCTGKVKGRKPAALGE |
| (without signal | PQPTKSLEENKSLKEQKKLNDSCFLKRLLQKIKTCWNKILMGTKEH |
| peptide) | |
| Cow IL-7 Protein | DCDISGKDGGAYQNVLMVNIDDLDNMINFDSNCLNNEPNFFKKHSCDDNKEAS |
| (SEQ ID NO: 84) | FLNRASRKLRQFLKMNISDDFKLHLSTVSQGTLTLLNCTSKGKGRKPPSLSEA |
| (without signal | QPTKNLEENKSSKEQKKQNDLCFLKILLQKIKTCWNKILRGIKEH |
| peptide) | |
| Sheep IL-7 Protein | DCDFSGKDGGAYQNVLMVSIDDLDNMINFDSNCLNNEPNFFKKHSCDDNKEAS |
| (SEQ ID NO: 85) | FLNRAARKLKQFLKMNISDDFKLHLSTVSQGTLTLLNCTSKGKGRKPPSLGEA |
| (without signal | QPTKNLEENKSLKEQRKQNDLCFLKILLQKIKTCWNKILRGITEH |
| peptide) | |
| Fc #1 (SEQ ID NO: | APTKAPDVFPIISGCRHPKDNSPVVLACLITGYHPTSVTVTWYMGTQSQPQRT |
| 7) | FPEIQRRDSYYMTSSQLSTPLQQWRQGEYKCVVQHTASKSKKEIFRWPESPKA |
| | QASSVPTAQPQAEGSLAKATTAPATTRNTGRGGEEKKKEKEKEEQEERETKTP |
| | ECPSHTQPLGVYLLTPAVQDLWLRDKATFTCFVVGSDLKDAHLTWEVAGKVPT |
| | GGVEEGLLERHSNGSQSQHSRLTLPRSLWNAGTSVTCTLNHPSLPPQRLMALR |
| | EPAAQAPVKLSLNLLASSDPPEAASWLLCEVSGFSPPNILLMWLEDQREVNTS |
| | GFAPARPPPQPGSTTFWAWSVLRVPAPPSPQPATYTCVVSHEDSRTLLNASRS |
| | LEVSYVTDHGPMK |
| Fc #2 (SEQ ID NO: | ASTKGPSVFPLAPCSRSTSESTAALGCLVKDYFPEPVTVSWNSGALTSGVHTF |
| 8) | PAVLQSSGLYSLSSVVTVPSSSLGTKTYTCNVDHKPSNTKVDKRVESKYGPPC |
| | PSCPAPEFLGGPSVFLFPPKPKDTLMISRTPEVTCVVVDVSQEDPEVQFNWYV |
| | DGVEVHNAKTKPREEQFNSTYRVVSVLTVLHQDWLNGKEYKCKVSNKGLPSSI |
| | EKTISKAKGQPREPQVYTLPPSQEEMTKNQVSLTCLVKGFYPSDIAVEWESNG |
| | QPENNYKTTPPVLDSDGSFFLYSRLTVDKSRWQEGNVFSCSVMHEALHNHYTQ |
| | KSLSLSLGK |
| Fc #3 (hyFc) | RNTGRGGEEKKKEKEKEEQEERETKTPECPSHTQPLGVFLFPPKPKDTLMISR |
| (SEQ ID NO: 9) | TPEVTCVVVDVSQEDPEVQFNWYVDGVEVHNAKTKPREEQFNSTYRVVSVLTV |
| | LHQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVMHEALHNHYTQKSLSLSLGK |
| HyFcM1 | RNTGRGGEEKKGGKEKEEQEERETKTPECPSHTQPLGVFLFPPKPKDTLMISR |
| (SEQ ID NO: 10) | TPEVTCVVVDVSQEDPEVQFNWYVDGVEVHNAKTKPREEQFNSTYRVVSVLTV |
| | LHQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVMHEALHNHYTQKSLSLSLGK |
| HyFcM2 | RNTGRGGEEKKGSKEKEEQEERETKTPECPSHTQPLGVFLFPPKPKDTLMISR |
| (SEQ ID NO: 11) | TPEVTCVVVDVSQEDPEVQFNWYVDGVEVHNAKTKPREEQFNSTYRVVSVLTV |
| | LHQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVMHEALHNHYTQKSLSLSLGK |
| HyFcM3 | RNTGRGGEEKKSGKEKEEQEERETKTPECPSHTQPLGVFLFPPKPKDTLMISR |
| (SEQ ID NO: 12) | TPEVTCVVVDVSQEDPEVQFNWYVDGVEVHNAKTKPREEQFNSTYRVVSVLTV |
| | LHQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVMHEALHNHYTQKSLSLSLGK |
| HyFcM4 | RNTGRGGEEKKSSKEKEEQEERETKTPECPSHTQPLGVFLFPPKPKDTLMISR |
| (SEQ ID NO: 13) | TPEVTCVVVDVSQEDPEVQFNWYVDGVEVHNAKTKPREEQFNSTYRVVSVLTV |
| | LHQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSRLTVDK |
| | SRWQEGNVFSCSVMHEALHNHYTQKSLSLSLGK |
| Non-lytic mouse Fc | ASAEPRGPTIKPCPPCKCPAPNLEGGPSVFIFPPKIKDVLMISLSPIVTCVVV |
| (SEQ ID NO: 14) | DVSEDDPDVQISWFVNNVEVHTAQTQTHREDYNSTLRVVSALPIQHQDWMSGK |
| | AFACAVNNKDLPAPIERTISKPKGSVRAPQVYVLPPPEEEMTKKQVTLTCMVT |
| | DFMPEDIYVEWTNNGKTELNYKNTEPVLDSDGSYFMYSKLRVEKKNWVERNSY |
| | SCSVVHEGLHNHHTTKSFSRTPGKGGGNSGS |
| IL-7 Fusion #1 | MDCDIEGKDGKQYESVLMVSIDQLLDSMKEIGSNCLNNEFNFFKRHICDANKE |
| (SEQ ID NO: 15) | GMFLFRAARKLRQFLKMNSTGDFDLHLLKVSEGTTILLNCTGQVKGRKPAALG |
| | EAQPTKSLEENKSLKEQKKLNDLCFLKRLLQEIKTCWNKILMGTKEH |
| IL-7 Fusion #2 | MMDCDIEGKDGKQYESVLMVSIDQLLDSMKEIGSNCLNNEFNFFKRHICDANK |
| (SEQ ID NO: 16) | EGMFLFRAARKLRQFLKMNSTGDFDLHLLKVSEGTTILLNCTGQVKGRKPAAL |
| | GEAQPTKSLEENKSLKEQKKLNDLCFLKRLLQEIKTCWNKILMGTKEH |
| IL-7 Fusion #3 | MMMDCDIEGKDGKQYESVLMVSIDQLLDSMKEIGSNCLNNEFNFFKRHICDAN |
| (SEQ ID NO: 17) | KEGMFLFRAARKLRQFLKMNSTGDFDLHLLKVSEGTTILLNCTGQVKGRKPAA |
| | LGEAQPTKSLEENKSLKEQKKLNDLCFLKRLLQEIKTCWNKILMGTKEH |
| IL-7 Fusion #4 | MGMDCDIEGKDGKQYESVLMVSIDQLLDSMKEIGSNCLNNEFNFFKRHICDAN |
| (SEQ ID NO: 18) | KEGMFLFRAARKLRQFLKMNSTGDFDLHLLKVSEGTTILLNCTGQVKGRKPAA |
| | LGEAQPTKSLEENKSLKEQKKLNDLCFLKRLLQEIKTCWNKILMGTKEH |
| IL-7 Fusion #5 | DDDDCDIEGKDGKQYESVLMVSIDQLLDSMKEIGSNCLNNEFNFFKRHICDAN |
| (SEQ ID NO: 19) | KEGMFLFRAARKLRQFLKMNSTGDFDLHLLKVSEGTTILLNCTGQVKGRKPAA |
| | LGEAQPTKSLEENKSLKEQKKLNDLCFLKRLLQEIKTCWNKILMGTKEH |
| IL-7 Fusion #6 | MMMMDCDIEGKDGKQYESVLMVSIDQLLDSMKEIGSNCLNNEFNFFKRHICDA |
| (SEQ ID NO: 20) | NKEGMFLFRAARKLRQFLKMNSTGDFDLHLLKVSEGTTILLNCTGQVKGRKPA |
| | ALGEAQPTKSLEENKSLKEQKKLNDLCFLKRLLQEIKTCWNKILMGTKEH |
| IL-7 Fusion #7 | MDCDIEGKDGKQYESVLMVSIDQLLDSMKEIGSNCLNNEFNFFKRHICDANKE |
| (SEQ ID NO: 21) | GMFLFRAARKLRQFLKMNSTGDFDLHLLKVSEGTTILLNCTGQVKGRKPAALG |
| | EAQPTKSLEENKSLKEQKKLNDLCFLKRLLQEIKTCWNKILMGTKEHRNTGRG |
| | GEEKKKEKEKEEQEERETKTPECPSHTQPLGVFLFPPKPKDTLMISRTPEVTC |
| | VVVDVSQEDPEVQFNWYVDGVEVHNAKTKPREEQFNSTYRVVSVLTVLHQDWL |
| | NGKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQEG |
| | NVFSCSVMHEALHNHYTQKSLSLSLGK |
| IL-7 Fusion #8 | MMDCDIEGKDGKQYESVLMVSIDQLLDSMKEIGSNCLNNEFNFFKRHICDANK |
| (SEQ ID NO: 22) | EGMFLFRAARKLRQFLKMNSTGDFDLHLLKVSEGTTILLNCTGQVKGRKPAAL |
| | GEAQPTKSLEENKSLKEQKKLNDLCFLKRLLQEIKTCWNKILMGTKEHRNTGR |
| | GGEEKKKEKEKEEQEERETKTPECPSHTQPLGVFLFPPKPKDTLMISRTPEVT |
| | CVVVDVSQEDPEVQFNWYVDGVEVHNAKTKPREEQFNSTYRVVSVLTVLHQDW |
| | LNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQE |
| | GNVFSCSVMHEALHNHYTQKSLSLSLGK |
| IL-7 Fusion #9 | MMMDCDIEGKDGKQYESVLMVSIDQLLDSMKEIGSNCLNNEFNFFKRHICDAN |
| (SEQ ID NO: 23) | KEGMFLFRAARKLRQFLKMNSTGDFDLHLLKVSEGTTILLNCTGQVKGRKPAA |
| | LGEAQPTKSLEENKSLKEQKKLNDLCFLKRLLQEIKTCWNKILMGTKEHRNTG |
| | RGGEEKKKEKEKEEQEERETKTPECPSHTQPLGVFLFPPKPKDTLMISRTPEV |
| | TCVVVDVSQEDPEVQFNWYVDGVEVHNAKTKPREEQFNSTYRVVSVLTVLHQD |
| | WLNGKEYKCKVSNKGLPSSIEKTISKAKGQPREPQVYTLPPSQEEMTKNQVSL |
| | TCLVKGFYPSDIAVEWESNGQPENNYKTTPPVLDSDGSFFLYSRLTVDKSRWQ |
| | EGNVFSCSVMHEALHNHYTQKSLSLSLGK |
| IL-7 Fusion #10 | MGMDCDIEGKDGKQYESVLMVSIDQLLDSMKEIGSNCLNNEFNFFKRHICDAN |
| (SEQ ID NO: 24) | KEGMFLFRAARKLRQFLKMNSTGDFDLHLLKVSEGTTILLNCTGQVKGRKPAA |
| | LGEAQPTKSLEENKSLKEQKKLNDLCFLKRLLQEIKTCWNKILMGTKEHRNTG |
| | RGGEEKKKEKEKEEQEERETKTPECPSHTQPLGVFLFPPKPKDTLMISRTPEV |
| | TCVVVDVSQEDPEVQFNWYVDGVEVHNAKTKPREEQFNSTYRVVSVLTVLHQD |
| | WLNGKEYKCKVSNKGLPSSIEKTISKAKGQPREPQVYTLPPSQEEMTKNQVSL |
| | TCLVKGFYPSDIAVEWESNGQPENNYKTTPPVLDSDGSFFLYSRLTVDKSRWQ |
| | EGNVFSCSVMHEALHNHYTQKSLSLSLGK |
| IL-7 Fusion #11 | MMMMDCDIEGKDGKQYESVLMVSIDQLLDSMKEIGSNCLNNEFNFFKRHICDA |
| (SEQ ID NO: 25) | NKEGMFLFRAARKLRQFLKMNSTGDFDLHLLKVSEGTTILLNCTGQVKGRKPA |
| | ALGEAQPTKSLEENKSLKEQKKLNDLCFLKRLLQEIKTCWNKILMGTKEHRNT |
| | GRGGEEKKKEKEKEEQEERETKTPECPSHTQPLGVFLFPPKPKDTLMISRTPE |
| | VTCVVVDVSQEDPEVQFNWYVDGVEVHNAKTKPREEQFNSTYRVVSVLTVLHQ |
| | DWLNGKEYKCKVSNKGLPSSIEKTISKAKGQPREPQVYTLPPSQEEMTKNQVS |
| | LTCLVKGFYPSDIAVEWESNGQPENNYKTTPPVLDSDGSFFLYSRLTVDKSRW |
| | QEGNVFSCSVMHEALHNHYTQKSLSLSLGK |
| IL-7 Fusion #12 | DCDIEGKDGKQYESVLMVSIDQLLDSMKEIGSNCLNNEFNFFKRHICDANKEG |
| (SEQ ID NO: 26) | MFLFRAARKLRQFLKMNSTGDFDLHLLKVSEGTTILLNCTGQVKGRKPAALGE |
| | AQPTKSLEENKSLKEQKKLNDLCFLKRLLQEIKTCWNKILMGTKEHRNTGRGG |
| | EEKKKEKEKEEQEERETKTPECPSHTQPLGVFLFPPKPKDTLMISRTPEVTCV |
| | VVDVSQEDPEVQFNWYVDGVEVHNAKTKPREEQFNSTYRVVSVLTVLHQDWLN |
| | GKEYKCKVSNKGLPSSIEKTISKAKGQPREPQVYTLPPSQEEMTKNQVSLTCL |
| | VKGFYPSDIAVEWESNGQPENNYKTTPPVLDSDGSFFLYSRLTVDKSRWQEGN |
| | VFSCSVMHEALHNHYTQKSLSLSLGK |
| IL-7 Fusion #13 | DCDIEGKDGKQYESVLMVSIDQLLDSMKEIGSNCLNNEFNFFKRHICDANKEG |
| (SEQ ID NO: 27) | MFLFRAARKLRQFLKMNSTGDFDLHLLKVSEGTTILLNCTGQVKGRKPAALGE |
| | AQPTKSLEENKSLKEQKKLNDLCFLKRLLQEIKTCWNKILMGTKEHASAEPRG |
| | PTIKPCPPCKCPAPNLEGGPSVFIFPPKIKDVLMISLSPIVTCVVVDVSEDDP |
| | DVQISWFVNNVEVHTAQTQTHREDYNSTLRVVSALPIQHQDWMSGKAFACAVN |
| | NKDLPAPIERTISKPKGSVRAPQVYVLPPPEEEMTKKQVTLTCMVTDFMPEDI |
| | YVEWTNNGKTELNYKNTEPVLDSDGSYFMYSKLRVEKKNWVERNSYSCSVVHE |
| | GLHNHHTTKSFSRTPGKGGGNSGS |
| IL-7 Sequence #1 | ATGTTCCACGTGAGCTTCAGGTACATCTTCGGCCTGCCACCCCTGATCCTGGT |
| (SEQ ID NO: 28) | GCTGCTGCCTGTGGCCAGCTCCGACTGCGACATCGAGGGAAAAGACGGCAAGC |
| | AGTACGAAAGCGTGCTGATGGTGTCCATCGACCAGCTGCTGGATTCTATGAAG |
| | GAGATTGGGAGTAACTGCCTGAACAATGAGTTCAACTTCTTCAAACGGCACAT |
| | TTGTGATGCCAACAAGGAGGGAATGTTCCTGTTTCGGGCCGCTAGAAAACTGA |
| | GGCAGTTCCTGAAGATGAACAGCACCGGAGACTTTGATCTGCATCTGCTGAAA |
| | GTGTCTGAGGGCACCACAATCCTGCTGAACTGCACTGGGCAGGTGAAAGGAAG |
| | GAAGCCTGCCGCTCTGGGAGAGGCTCAGCCAACCAAGTCACTGGAGGAAAACA |
| | AAAGCCTGAAGGAACAGAAGAAACTGAATGACCTGTGCTTTCTGAAACGGCTG |
| | CTGCAGGAGATCAAAACATGTTGGAACAAGATTCTGATGGGCACAAAGGAACA |
| | C |
| IL-7 Sequence #2 | ATGTTCCACGTGAGCTTCAGATACATCTTCGGCCTGCCCCCCCTGATCCTGGT |
| (SEQ ID NO: 29) | GCTGCTGCCCGTGGCCAGCAGCATGGACTGCGACATCGAGGGCAAGGACGGCA |
| | AGCAGTACGAGAGCGTGCTGATGGTGAGCATCGACCAGCTGCTGGACAGCATG |
| | AAGGAGATCGGCAGCAACTGCCTGAACAACGAGTTCAACTTCTTCAAGAGACA |
| | CATCTGCGACGCCAACAAGGAGGGCATGTTCCTGTTCAGAGCCGCCAGAAAGC |
| | TGAGACAGTTCCTGAAGATGAACAGCACCGGCGACTTCGACCTGCACCTGCTG |
| | AAGGTGAGCGAGGGCACAACCATCCTGCTGAACTGCACCGGCCAGGTGAAGGG |
| | CAGAAAGCCCGCCGCCCTGGGCGAGGCCCAGCCCACCAAGAGCCTGGAGGAGA |
| | ACAAGAGCCTGAAGGAGCAGAAGAAGCTGAACGACCTGTGCTTCCTGAAGAGA |
| | CTGCTGCAGGAGATCAAGACCTGCTGGAACAAGATCCTGATGGGCACCAAGGA |
| | GCAC |
| IL-7 Sequence #3 | ATGTTCCACGTGAGCTTCAGATACATCTTCGGCCTGCCCCCCCTGATCCTGGT |
| (SEQ ID NO: 30) | GCTGCTGCCCGTGGCCAGCAGCATGATGGACTGCGACATCGAGGGCAAGGACG |
| | GCAAGCAGTACGAGAGCGTGCTGATGGTGAGCATCGACCAGCTGCTGGACAGC |
| | ATGAAGGAGATCGGCAGCAACTGCCTGAACAACGAGTTCAACTTCTTCAAGAG |
| | ACACATCTGCGACGCCAACAAGGAGGGCATGTTCCTGTTCAGAGCCGCCAGAA |
| | AGCTGAGACAGTTCCTGAAGATGAACAGCACCGGCGACTTCGACCTGCACCTG |
| | CTGAAGGTGAGCGAGGGCACAACCATCCTGCTGAACTGCACCGGCCAGGTGAA |
| | GGGCAGAAAGCCCGCCGCCCTGGGCGAGGCCCAGCCCACCAAGAGCCTGGAGG |
| | AGAACAAGAGCCTGAAGGAGCAGAAGAAGCTGAACGACCTGTGCTTCCTGAAG |
| | AGACTGCTGCAGGAGATCAAGACCTGCTGGAACAAGATCCTGATGGGCACCAA |
| | GGAGCAC |
| IL-7 Sequence #4 | ATGTTCCACGTGAGCTTCAGATACATCTTCGGCCTGCCCCCCCTGATCCTGGT |
| (SEQ ID NO: 31) | GCTGCTGCCCGTGGCCAGCAGCATGATGATGGACTGCGACATCGAGGGCAAGG |
| | ACGGCAAGCAGTACGAGAGCGTGCTGATGGTGAGCATCGACCAGCTGCTGGAC |
| | AGCATGAAGGAGATCGGCAGCAACTGCCTGAACAACGAGTTCAACTTCTTCAA |
| | GAGACACATCTGCGACGCCAACAAGGAGGGCATGTTCCTGTTCAGAGCCGCCA |
| | GAAAGCTGAGACAGTTCCTGAAGATGAACAGCACCGGCGACTTCGACCTGCAC |
| | CTGCTGAAGGTGAGCGAGGGCACAACCATCCTGCTGAACTGCACCGGCCAGGT |
| | GAAGGGCAGAAAGCCCGCCGCCCTGGGCGAGGCCCAGCCCACCAAGAGCCTGG |
| | AGGAGAACAAGAGCCTGAAGGAGCAGAAGAAGCTGAACGACCTGTGCTTCCTG |
| | AAGAGACTGCTGCAGGAGATCAAGACCTGCTGGAACAAGATCCTGATGGGCAC |
| | CAAGGAGCAC |
| IL-7 Sequence #5 | ATGTTCCACGTGAGCTTCAGGTACATCTTCGGCCTGCCACCCCTGATCCTGGT |
| (SEQ ID NO: 32) | GCTGCTGCCTGTGGCCAGCTCCATGGGGATGGACTGCGACATCGAGGGAAAAG |
| | ACGGCAAGCAGTACGAAAGCGTGCTGATGGTGTCCATCGACCAGCTGCTGGAT |
| | TCTATGAAGGAGATTGGGAGTAACTGCCTGAACAATGAGTTCAACTTCTTCAA |
| | ACGGCACATTTGTGATGCCAACAAGGAGGGAATGTTCCTGTTTCGGGCCGCTA |
| | GAAAACTGAGGCAGTTCCTGAAGATGAACAGCACCGGAGACTTTGATCTGCAT |
| | CTGCTGAAAGTGTCTGAGGGCACCACAATCCTGCTGAACTGCACTGGGCAGGT |
| | GAAAGGAAGGAAGCCTGCCGCTCTGGGAGAGGCTCAGCCAACCAAGTCACTGG |
| | AGGAAAACAAAAGCCTGAAGGAACAGAAGAAACTGAATGACCTGTGCTTTCTG |
| | AAACGGCTGCTGCAGGAGATCAAAACATGTTGGAACAAGATTCTGATGGGCAC |
| | CAAGGAGCAC |
| IL-7 Sequence #6 | ATGTTCCACGTGAGCTTCAGATACATCTTCGGCCTGCCCCCCCTGATCCTGGT |
| (SEQ ID NO: 33) | GCTGCTGCCCGTGGCCAGCAGCGACGATGACGACTGCGACATCGAGGGCAAGG |
| | ACGGCAAGCAGTACGAGAGCGTGCTGATGGTGAGCATCGACCAGCTGCTGGAC |
| | AGCATGAAGGAGATCGGCAGCAACTGCCTGAACAACGAGTTCAACTTCTTCAA |
| | GAGACACATCTGCGACGCCAACAAGGAGGGCATGTTCCTGTTCAGAGCCGCCA |
| | GAAAGCTGAGACAGTTCCTGAAGATGAACAGCACCGGCGACTTCGACCTGCAC |
| | CTGCTGAAGGTGAGCGAGGGCACAACCATCCTGCTGAACTGCACCGGCCAGGT |
| | GAAGGGCAGAAAGCCCGCCGCCCTGGGCGAGGCCCAGCCCACCAAGAGCCTGG |
| | AGGAGAACAAGAGCCTGAAGGAGCAGAAGAAGCTGAACGACCTGTGCTTCCTG |
| | AAGAGACTGCTGCAGGAGATCAAGACCTGCTGGAACAAGATCCTGATGGGCAC |
| | CAAGGAGCAC |
| IL-7 Sequence #7 | ATGTTCCACGTGAGCTTCAGATACATCTTCGGCCTGCCCCCCCTGATCCTGGT |
| (SEQ ID NO: 34) | GCTGCTGCCCGTGGCCAGCAGCATGATGATGATGGACTGCGACATCGAGGGCA |
| | AGGACGGCAAGCAGTACGAGAGCGTGCTGATGGTGAGCATCGACCAGCTGCTG |
| | GACAGCATGAAGGAGATCGGCAGCAACTGCCTGAACAACGAGTTCAACTTCTT |
| | CAAGAGACACATCTGCGACGCCAACAAGGAGGGCATGTTCCTGTTCAGAGCCG |
| | CCAGAAAGCTGAGACAGTTCCTGAAGATGAACAGCACCGGCGACTTCGACCTG |
| | CACCTGCTGAAGGTGAGCGAGGGCACAACCATCCTGCTGAACTGCACCGGCCA |
| | GGTGAAGGGCAGAAAGCCCGCCGCCCTGGGCGAGGCCCAGCCCACCAAGAGCC |
| | TGGAGGAGAACAAGAGCCTGAAGGAGCAGAAGAAGCTGAACGACCTGTGCTTC |
| | CTGAAGAGACTGCTGCAGGAGATCAAGACCTGCTGGAACAAGATCCTGATGGG |
| | CACCAAGGAGCAC |
| IL-7 Sequence #8 | ATGTTCCACGTGAGCTTCAGATACATCTTCGGCCTGCCCCCCCTGATCCTGGT |
| (SEQ ID NO: 35) | GCTGCTGCCCGTGGCCAGCAGCATGGACTGCGACATCGAGGGCAAGGACGGCA |
| | AGCAGTACGAGAGCGTGCTGATGGTGAGCATCGACCAGCTGCTGGACAGCATG |
| | AAGGAGATCGGCAGCAACTGCCTGAACAACGAGTTCAACTTCTTCAAGAGACA |
| | CATCTGCGACGCCAACAAGGAGGGCATGTTCCTGTTCAGAGCCGCCAGAAAGC |
| | TGAGACAGTTCCTGAAGATGAACAGCACCGGCGACTTCGACCTGCACCTGCTG |
| | AAGGTGAGCGAGGGCACAACCATCCTGCTGAACTGCACCGGCCAGGTGAAGGG |
| | CAGAAAGCCCGCCGCCCTGGGCGAGGCCCAGCCCACCAAGAGCCTGGAGGAGA |
| | ACAAGAGCCTGAAGGAGCAGAAGAAGCTGAACGACCTGTGCTTCCTGAAGAGA |
| | CTGCTGCAGGAGATCAAGACCTGCTGGAACAAGATCCTGATGGGCACCAAGGA |
| | GCACAGGAACACAGGCAGAGGCGGCGAGGAGAAGAAGAAGGAGAAGGAGAAGG |
| | AGGAGCAGGAGGAAAGAGAGACCAAGACCCCCGAGTGCCCCAGCCACACCCAG |
| | CCCCTGGGCGTGTTCCTGTTCCCTCCCAAGCCCAAGGACACCCTGATGATCAG |
| | CAGAACCCCCGAGGTGACCTGCGTGGTCGTGGATGTGAGCCAGGAAGATCCCG |
| | AAGTGCAGTTCAACTGGTACGTGGATGGCGTGGAAGTGCACAACGCCAAGACC |
| | AAGCCCAGAGAAGAGCAGTTCAACTCCACCTACAGAGTGGTGAGCGTGCTGAC |
| | CGTGCTGCACCAGGACTGGCTGAACGGCAAGGAGTACAAGTGCAAGGTGTCCA |
| | ACAAAGGCCTGCCCAGCTCCATCGAGAAGACCATCAGCAAAGCCAAAGGCCAG |
| | CCCAGAGAACCCCAGGTGTACACCCTGCCTCCCAGCCAGGAAGAGATGACCAA |
| | GAACCAGGTGTCCCTGACCTGCCTGGTGAAAGGCTTCTACCCCAGCGACATCG |
| | CCGTGGAGTGGGAAAGCAACGGCCAGCCCGAGAACAATTACAAGACAACCCCT |
| | CCCGTGCTGGATAGCGATGGCAGCTTCTTTCTGTACAGCAGACTGACCGTGGA |
| | CAAGAGCAGATGGCAGGAAGGCAACGTGTTCAGCTGCAGCGTGATGCACGAAG |
| | CCCTGCACAACCACTACACCCAGAAGAGCCTGTCCCTGAGCCTGGGCAAGTGA |
| | CTCGAGTCTAGA |
| IL-7 Sequence #9 | ATGTTCCACGTGAGCTTCAGATACATCTTCGGCCTGCCCCCCCTGATCCTGGT |
| (SEQ ID NO: 36) | GCTGCTGCCCGTGGCCAGCAGCATGATGGACTGCGACATCGAGGGCAAGGACG |
| | GCAAGCAGTACGAGAGCGTGCTGATGGTGAGCATCGACCAGCTGCTGGACAGC |
| | ATGAAGGAGATCGGCAGCAACTGCCTGAACAACGAGTTCAACTTCTTCAAGAG |
| | ACACATCTGCGACGCCAACAAGGAGGGCATGTTCCTGTTCAGAGCCGCCAGAA |
| | AGCTGAGACAGTTCCTGAAGATGAACAGCACCGGCGACTTCGACCTGCACCTG |
| | CTGAAGGTGAGCGAGGGCACAACCATCCTGCTGAACTGCACCGGCCAGGTGAA |
| | GGGCAGAAAGCCCGCCGCCCTGGGCGAGGCCCAGCCCACCAAGAGCCTGGAGG |
| | AGAACAAGAGCCTGAAGGAGCAGAAGAAGCTGAACGACCTGTGCTTCCTGAAG |
| | AGACTGCTGCAGGAGATCAAGACCTGCTGGAACAAGATCCTGATGGGCACCAA |
| | GGAGCACAGGAACACAGGCAGAGGCGGCGAGGAGAAGAAGAAGGAGAAGGAGA |
| | AGGAGGAGCAGGAGGAAAGAGAGACCAAGACCCCCGAGTGCCCCAGCCACACC |
| | CAGCCCCTGGGCGTGTTCCTGTTCCCTCCCAAGCCCAAGGACACCCTGATGAT |
| | CAGCAGAACCCCCGAGGTGACCTGCGTGGTCGTGGATGTGAGCCAGGAAGATC |
| | CCGAAGTGCAGTTCAACTGGTACGTGGATGGCGTGGAAGTGCACAACGCCAAG |
| | ACCAAGCCCAGAGAAGAGCAGTTCAACTCCACCTACAGAGTGGTGAGCGTGCT |
| | GACCGTGCTGCACCAGGACTGGCTGAACGGCAAGGAGTACAAGTGCAAGGTGT |
| | CCAACAAAGGCCTGCCCAGCTCCATCGAGAAGACCATCAGCAAAGCCAAAGGC |
| | CAGCCCAGAGAACCCCAGGTGTACACCCTGCCTCCCAGCCAGGAAGAGATGAC |
| | CAAGAACCAGGTGTCCCTGACCTGCCTGGTGAAAGGCTTCTACCCCAGCGACA |
| | TCGCCGTGGAGTGGGAAAGCAACGGCCAGCCCGAGAACAATTACAAGACAACC |
| | CCTCCCGTGCTGGATAGCGATGGCAGCTTCTTTCTGTACAGCAGACTGACCGT |
| | GGACAAGAGCAGATGGCAGGAAGGCAACGTGTTCAGCTGCAGCGTGATGCACG |
| | AAGCCCTGCACAACCACTACACCCAGAAGAGCCTGTCCCTGAGCCTGGGCAAG |
| IL-7 Sequence #10 | ATGTTCCACGTGAGCTTCAGATACATCTTCGGCCTGCCCCCCCTGATCCTGGT |
| (SEQ ID NO: 37) | GCTGCTGCCCGTGGCCAGCAGCATGATGATGGACTGCGACATCGAGGGCAAGG |
| | ACGGCAAGCAGTACGAGAGCGTGCTGATGGTGAGCATCGACCAGCTGCTGGAC |
| | AGCATGAAGGAGATCGGCAGCAACTGCCTGAACAACGAGTTCAACTTCTTCAA |
| | GAGACACATCTGCGACGCCAACAAGGAGGGCATGTTCCTGTTCAGAGCCGCCA |
| | GAAAGCTGAGACAGTTCCTGAAGATGAACAGCACCGGCGACTTCGACCTGCAC |
| | CTGCTGAAGGTGAGCGAGGGCACAACCATCCTGCTGAACTGCACCGGCCAGGT |
| | GAAGGGCAGAAAGCCCGCCGCCCTGGGCGAGGCCCAGCCCACCAAGAGCCTGG |
| | AGGAGAACAAGAGCCTGAAGGAGCAGAAGAAGCTGAACGACCTGTGCTTCCTG |
| | AAGAGACTGCTGCAGGAGATCAAGACCTGCTGGAACAAGATCCTGATGGGCAC |
| | CAAGGAGCACAGGAACACAGGCAGAGGCGGCGAGGAGAAGAAGAAGGAGAAGG |
| | AGAAGGAGGAGCAGGAGGAAAGAGAGACCAAGACCCCCGAGTGCCCCAGCCAC |
| | ACCCAGCCCCTGGGCGTGTTCCTGTTCCCTCCCAAGCCCAAGGACACCCTGAT |
| | GATCAGCAGAACCCCCGAGGTGACCTGCGTGGTCGTGGATGTGAGCCAGGAAG |
| | ATCCCGAAGTGCAGTTCAACTGGTACGTGGATGGCGTGGAAGTGCACAACGCC |
| | AAGACCAAGCCCAGAGAAGAGCAGTTCAACTCCACCTACAGAGTGGTGAGCGT |
| | GCTGACCGTGCTGCACCAGGACTGGCTGAACGGCAAGGAGTACAAGTGCAAGG |
| | TGTCCAACAAAGGCCTGCCCAGCTCCATCGAGAAGACCATCAGCAAAGCCAAA |
| | GGCCAGCCCAGAGAACCCCAGGTGTACACCCTGCCTCCCAGCCAGGAAGAGAT |
| | GACCAAGAACCAGGTGTCCCTGACCTGCCTGGTGAAAGGCTTCTACCCCAGCG |
| | ACATCGCCGTGGAGTGGGAAAGCAACGGCCAGCCCGAGAACAATTACAAGACA |
| | ACCCCTCCCGTGCTGGATAGCGATGGCAGCTTCTTTCTGTACAGCAGACTGAC |
| | CGTGGACAAGAGCAGATGGCAGGAAGGCAACGTGTTCAGCTGCAGCGTGATGC |
| | ACGAAGCCCTGCACAACCACTACACCCAGAAGAGCCTGTCCCTGAGCCTGGGC |
| | AAG |
| IL-7 Sequence #11 | ATGTTCCACGTGAGCTTCAGGTACATCTTCGGCCTGCCACCCCTGATCCTGGT |
| (SEQ ID NO: 38) | GCTGCTGCCTGTGGCCAGCTCCATGGGGATGGACTGCGACATCGAGGGAAAAG |
| | ACGGCAAGCAGTACGAAAGCGTGCTGATGGTGTCCATCGACCAGCTGCTGGAT |
| | TCTATGAAGGAGATTGGGAGTAACTGCCTGAACAATGAGTTCAACTTCTTCAA |
| | ACGGCACATTTGTGATGCCAACAAGGAGGGAATGTTCCTGTTTCGGGCCGCTA |
| | GAAAACTGAGGCAGTTCCTGAAGATGAACAGCACCGGAGACTTTGATCTGCAT |
| | CTGCTGAAAGTGTCTGAGGGCACCACAATCCTGCTGAACTGCACTGGGCAGGT |
| | GAAAGGAAGGAAGCCTGCCGCTCTGGGAGAGGCTCAGCCAACCAAGTCACTGG |
| | AGGAAAACAAAAGCCTGAAGGAACAGAAGAAACTGAATGACCTGTGCTTTCTG |
| | AAACGGCTGCTGCAGGAGATCAAAACATGTTGGAACAAGATTCTGATGGGCAC |
| | AAAGGAACACCGCAATACTGGGCGGGGCGGGGAGGAAAAGAAAAAGGAGAAGG |
| | AAAAGGAGGAACAGGAGGAAAGAGAGACTAAGACCCCAGAATGTCCCAGCCAT |
| | ACTCAGCCCCTGGGGGTGTTCCTGTTTCCCCCTAAACCTAAGGATACCCTGAT |
| | GATCAGCAGGACACCCGAGGTGACCTGCGTGGTCGTGGATGTGAGCCAGGAAG |
| | ATCCCGAAGTGCAGTTCAACTGGTACGTGGATGGCGTGGAAGTGCACAACGCC |
| | AAGACCAAGCCCAGAGAAGAGCAGTTCAACTCCACCTACAGAGTGGTGAGCGT |
| | GCTGACCGTGCTGCACCAGGACTGGCTGAACGGCAAGGAGTACAAGTGCAAGG |
| | TGTCCAACAAAGGCCTGCCCAGCTCCATCGAGAAGACCATCAGCAAAGCCAAA |
| | GGCCAGCCCAGAGAACCCCAGGTGTACACCCTGCCTCCCAGCCAGGAAGAGAT |
| | GACCAAGAACCAGGTGTCCCTGACCTGCCTGGTGAAAGGCTTCTACCCCAGCG |
| | ACATCGCCGTGGAGTGGGAAAGCAACGGCCAGCCCGAGAACAATTACAAGACA |
| | ACCCCTCCCGTGCTGGATAGCGATGGCAGCTTCTTTCTGTACAGCAGACTGAC |
| | CGTGGACAAGAGCAGATGGCAGGAAGGCAACGTGTTCAGCTGCAGCGTGATGC |
| | ACGAAGCCCTGCACAACCACTACACCCAGAAGAGCCTGTCCCTGAGCCTGGGC |
| | AAG |
| IL-7 Sequence #12 | ATGTTCCACGTGAGCTTCAGATACATCTTCGGCCTGCCCCCCCTGATCCTGGT |
| (SEQ ID NO: 39) | GCTGCTGCCCGTGGCCAGCAGCATGATGATGATGGACTGCGACATCGAGGGCA |
| | AGGACGGCAAGCAGTACGAGAGCGTGCTGATGGTGAGCATCGACCAGCTGCTG |
| | GACAGCATGAAGGAGATCGGCAGCAACTGCCTGAACAACGAGTTCAACTTCTT |
| | CAAGAGACACATCTGCGACGCCAACAAGGAGGGCATGTTCCTGTTCAGAGCCG |
| | CCAGAAAGCTGAGACAGTTCCTGAAGATGAACAGCACCGGCGACTTCGACCTG |
| | CACCTGCTGAAGGTGAGCGAGGGCACAACCATCCTGCTGAACTGCACCGGCCA |
| | GGTGAAGGGCAGAAAGCCCGCCGCCCTGGGCGAGGCCCAGCCCACCAAGAGCC |
| | TGGAGGAGAACAAGAGCCTGAAGGAGCAGAAGAAGCTGAACGACCTGTGCTTC |
| | CTGAAGAGACTGCTGCAGGAGATCAAGACCTGCTGGAACAAGATCCTGATGGG |
| | CACCAAGGAGCACAGGAACACAGGCAGAGGCGGCGAGGAGAAGAAGAAGGAGA |
| | AGGAGAAGGAGGAGCAGGAGGAAAGAGAGACCAAGACCCCCGAGTGCCCCAGC |
| | CACACCCAGCCCCTGGGCGTGTTCCTGTTCCCTCCCAAGCCCAAGGACACCCT |
| | GATGATCAGCAGAACCCCCGAGGTGACCTGCGTGGTCGTGGATGTGAGCCAGG |
| | AAGATCCCGAAGTGCAGTTCAACTGGTACGTGGATGGCGTGGAAGTGCACAAC |
| | GCCAAGACCAAGCCCAGAGAAGAGCAGTTCAACTCCACCTACAGAGTGGTGAG |
| | CGTGCTGACCGTGCTGCACCAGGACTGGCTGAACGGCAAGGAGTACAAGTGCA |
| | AGGTGTCCAACAAAGGCCTGCCCAGCTCCATCGAGAAGACCATCAGCAAAGCC |
| | AAAGGCCAGCCCAGAGAACCCCAGGTGTACACCCTGCCTCCCAGCCAGGAAGA |
| | GATGACCAAGAACCAGGTGTCCCTGACCTGCCTGGTGAAAGGCTTCTACCCCA |
| | GCGACATCGCCGTGGAGTGGGAAAGCAACGGCCAGCCCGAGAACAATTACAAG |
| | ACAACCCCTCCCGTGCTGGATAGCGATGGCAGCTTCTTTCTGTACAGCAGACT |
| | GACCGTGGACAAGAGCAGATGGCAGGAAGGCAACGTGTTCAGCTGCAGCGTGA |
| | TGCACGAAGCCCTGCACAACCACTACACCCAGAAGAGCCTGTCCCTGAGCCTG |
| | GGCAAG |
| Oligopeptide- | MMMM |
| MMMM (SEQ ID | |
| NO: 40) | |

What is claimed is:

1. A method of treating a tumor in a subject in need thereof, comprising administering to the subject an anti-cancer treatment, wherein a tumor sample obtained from the subject comprises an O⁶-methylguanine-DNA methyltransferase (MGMT) promoter which is unmethylated, and wherein the anti-cancer treatment comprises an interleukin-7 (IL-7) protein.

2. The method of claim 1, comprising, prior to the administering, determining a methylation status of the MGMT promoter in the tumor sample obtained from the subject. 3 A method of identifying a subject suitable for an anti-cancer treatment, comprising determining a methylation status of an unmethylated O⁶-methylguanine-DNA methyltransferase (MGMT) promoter in a tumor sample obtained from the subject, wherein the subject is suitable for the anti-cancer treatment if the MGMT promoter is unmethylated; and wherein the anti-cancer treatment comprises an interleukin-7 (IL-7) protein.

4. The method of claim 3, further comprising administering the anti-cancer treatment to the subject who has been identified as being suitable for the anti-cancer treatment.

5. A method of increasing an anti-tumor immune response in a subject in need thereof, comprising administering to the subject an anti-cancer treatment, wherein the subject has a tumor which comprises an O⁶-methylguanine-DNA methyltransferase (MGMT) promoter that is unmethylated, and wherein the anti-cancer treatment comprises an interleukin-7 (IL-7) protein. 6 The method of claim 5, comprising, prior to the administering, determining a methylation status of the MGMT promoter in a tumor sample obtained from the subject.

7. The method of claim 6, wherein, after the administering, the anti-tumor immune response in the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold, as compared to a reference subject (e.g., the subject prior to the administering).

8. The method of any one of claims 1, 2, and 4 to 7, wherein, after the administering, a median progression-free survival (mPFS) of the subject is increased.

9. The method of claim 8, wherein the mPFS of the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold, as compared to a reference subject (e.g., the subject prior to the administering).

10. The method of claim 8 or 9, wherein, after the administering, the mPFS of the subject is at least about 8 months, at least about 9 months, at least about 10 months, at least about 11 months, or at least about 12 months.

11. The method of any one of claims 1, 2, and 4 to 10, wherein, after the administering, a median overall survival (mOS) of the subject is increased.

12. The method of claim 11, wherein the mOS of the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold, as compared to a reference subject (e.g., the subject prior to the administering).

13. The method of claim 11 or 12, wherein, after the administering, the mOS of the subject is at least about 12 months, at least about 13 months, at least about 14 months, at least about 15 months, at least about 16 months, at least about 17 months, at least about 18 months, at least about 19 months, or at least about 20 months.

14. The method of any one of claims 1, 2, and 4 to 13, wherein, after the administering, an absolute lymphocyte count (ALC) of the subject is increased.

15. The method of claim 14, wherein the ALC of the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold, as compared to a reference subject (e.g., the subject prior to the administering).

16. The method of claim 14 or 15, wherein the increase in the ALC persists in the subject for at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, or at least about 15 weeks.

17. The method of any one of claims 1, 2, and 4 to 16, wherein, after the administering, the number of tumor-infiltrating lymphocytes (TILs) in the tumor of the subject is increased.

18. The method of claim 17, wherein the number of TILs in the tumor of the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold, as compared to a reference subject (e.g., the subject prior to the administering).

19. The method of any one of claims 1 to 18, wherein the anti-cancer treatment comprises an additional therapeutic agent.

20. The method of claim 19, wherein the additional therapeutic agent comprises a standard of care (SOC).

21. The method of claim 19 or 20, wherein the additional therapeutic agent comprises a radiation therapy (RT), chemotherapy, hormone therapy, immunotherapy, photodynamic therapy, stem cell transplant, or combinations thereof.

22. The method of claim 21, wherein the additional therapeutic agent comprises a chemotherapy.

23. The method of claim 21 or 22, wherein the chemotherapy comprises temozolomide (TMZ).

24. The method of any one of claims 21 to 23, wherein the immunotherapy comprises an immune checkpoint inhibitor, immune checkpoint activator, adoptive cell therapy, or combinations thereof.

25. The method of claim 24, wherein the immune checkpoint inhibitor comprises a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or a combination thereof.

26. The method of claim 24 or 25, wherein the immune checkpoint activator comprises an OX40 agonist (e.g., anti-OX40 antibody), LAG-3 agonist (e.g. anti-LAG-3 antibody), 4-1BB (CD137) agonist (e.g., anti-CD137 antibody), GITR agonist (e.g., anti-GITR antibody), or a combination thereof.

27. The method of any one of claims 1 to 26, wherein the tumor comprises a glioma. 28 The method of claim 27, wherein the glioma comprises a high-grade glioma (HGG). 29 The method of claim 28, wherein the HGG comprises a glioblastoma (GBM), anaplastic astrocytoma, or both.

30. The method of any one of claims 1 to 29, wherein the tumor is newly diagnosed.

31. The method of any one of claims 2 to 4 and 6 to 30, wherein the methylation status of the MGMT promoter is determined by a methylation specific PCR (MS-PCR), pyrosequencing, high resolution melting, microarray (e.g., Infinium MethylationEPIC), immunohistochemistry (IHC), multiplex ligation-dependent probe amplification (MPLA), or combinations thereof.

32. The method of any one of claims 1 to 31, wherein the IL-7 protein is not a wild-type IL-7 protein.

33. The method of any one of claims 1 to 32, wherein the IL-7 protein comprises an oligopeptide consisting of 1 to 10 amino acid residues.

34. The method of claim 33, wherein the oligopeptide comprises methionine (M), glycine (G), methionine-methionine (MM), glycine-glycine (GG), methionine-glycine (MG), glycine-methionine (GM), methionine-methionine-methionine (MMM), methionine-methionine-glycine (MMG), methionine-glycine-methionine (MGM), glycine-methionine-methionine (GMM), methionine-glycine-glycine (MGG), glycine-methionine-glycine (GMG), glycine-glycine-methionine (GGM), glycine-glycine-glycine (GGG), methionine-glycine-glycine-methionine (MGGM) (SEQ ID NO: 41), methionine-methionine-glycine-glycine (MMGG) (SEQ ID NO: 42), glycine-glycine-methionine-methionine (GGMM) (SEQ ID NO: 43), methionine-glycine-methionine-glycine (MGMG) (SEQ ID NO: 44), glycine-methionine-methionine-glycine (GMMG) (SEQ ID NO: 45), glycine-glycine-glycine-methionine (GGGM) (SEQ ID NO: 46), methionine-glycine-glycine-glycine (MGGG) (SEQ ID NO: 47), glycine-methionine-glycine-glycine (GMGG) (SEQ ID NO: 48), glycine-glycine-methionine-glycine (GGMG) (SEQ ID NO: 49), glycine-glycine-methionine-methionine-methionine (GGMMM) (SEQ ID NO: 50), glycine-glycine-glycine-methionine-methionine (GGGMM) (SEQ ID NO: 51), glycine-glycine-glycine-glycine-methionine (GGGGM) (SEQ ID NO: 52), methionine-glycine-methionine-methionine-methionine (MGMMM) (SEQ ID NO: 53), methionine-glycine-glycine-methionine-methionine (MGGMM) (SEQ ID NO: 54), methionine-glycine-glycine-glycine-methionine (MGGGM) (SEQ ID NO: 55), methionine-methionine-glycine-methionine-methionine (MMGMM) (SEQ ID NO: 56, methionine-methionine-glycine-glycine-methionine (MMGGM) (SEQ ID NO: 57), methionine-methionine-glycine-glycine-glycine (MMGGG) (SEQ ID NO: 58), methionine-methionine-methionine-glycine-methionine (MMMGM) (SEQ ID NO: 59), methionine-glycine-methionine-glycine-methionine (MGMGM) (SEQ ID NO: 60), glycine-methionine-glycine-methionine-glycine (GMGMG) (SEQ ID NO: 61), glycine-methionine-methionine-methionine-glycine (GMMMG) (SEQ ID NO: 62), glycine-glycine-methionine-glycine-methionine (GGMGM) (SEQ ID NO: 63), glycine-glycine-methionine-methionine-glycine (GGMMG) (SEQ ID NO: 64), glycine-methionine-methionine-glycine-methionine (GMMGM) (SEQ ID NO: 65), methionine-glycien-methionine-methionine-glycine (MGMMG) (SEQ ID NO: 66), glycine-methionine-glycine-glycine-methionine (GMGGM) (SEQ ID NO: 67), methionine-methionine-glycine-methionine-glycine (MMGMG) (SEQ ID NO: 68), glycine-methionine-methionine-glycine-glycine (GMMGG) (SEQ ID NO: 69), glycine-methionine-glycine-glycine-glycine (GMGGG) (SEQ ID NO: 70), glycine-glycine-methionine-glycine-glycine (GGMGG) (SEQ ID NO: 71), glycine-glycine-glycine-glycine-glycine (GGGGG) (SEQ ID NO: 72), or combinations thereof.

35. The method of claim 34, wherein the oligopeptide is methionine-glycine-methionine (MGM).

36. The method of any one of claims 1 to 35, wherein the IL-7 protein comprises a half-life extending moiety.

37. The method of claim 36, wherein the half-life extending moiety comprises an Fc, albumin, an albumin-binding polypeptide, Pro/Ala/Ser (PAS), a C-terminal peptide (CTP) of the β subunit of human chorionic gonadotropin, polyethylene glycol (PEG), long unstructured hydrophilic sequences of amino acids (XTEN), hydroxyethyl starch (HES), an albumin-binding small molecule, or a combination thereof.

38. The method of claim 37, wherein the half-life extending moiety is a Fc.

39. The method of claim 38, wherein the Fc is a hybrid Fc, comprising a hinge region, a CH2 domain, and a CH3 domain,
wherein the hinge region comprises a human IgD hinge region,
wherein the CH2 domain comprises a part of human IgD CH2 domain and a part of human IgG4 CH2 domain, and
wherein the CH3 domain comprises a part of human IgG4 CH3 domain.

40. The method of any one of claims 1 to 39, wherein the IL-7 protein comprises an amino acid sequence having a sequence identity of at least about 70%, at least about 75%, at least about 80%, at least about 85%, at least about 90%, at least about 95%, at least about 96%, at least about 97%, at least about 98%, at least about 99%, or about 100% to SEQ ID NOs: 1-6, 80-85, and 15-27.

41. The method of any one of claims 1, 2, and 4 to 40, wherein the IL-7 protein is administered at a dose of greater than about 30 μg/kg, greater than about 60 μg/kg, greater than about 90 μg/kg, greater than about 120 μg/kg, greater than about 150 μg/kg, greater than about 180 μg/kg, greater than about 210 μg/kg, greater than about 240 μg/kg, greater than about 270 μg/kg, greater than about 300 μg/kg, greater than about 400 μg/kg, greater than about 500 μg/kg, greater than about 600 μg/kg, greater than about 700 μg/kg, greater than about 800 μg/kg, greater than about 900 μg/kg, greater than about 1,000 μg/kg, greater than about 1,100 μg/kg, greater than about 1,200 μg/kg, greater than about 1,300 μg/kg, greater than about 1,400 μg/kg, greater than about 1,500 μg/kg, greater than about 1,600 μg/kg, greater than about 1,700 μg/kg, greater than about 1,800 μg/kg, greater than about 1,900 μg/kg, or greater than about 2,000 μg/kg.

42. The method of any one of claims 1, 2, and 4 to 41, wherein the IL-7 protein is administered at a dose of between about 60 μg/kg and about 1,200 μg/kg, between about 120 μg/kg and about 1,200 μg/kg, between about 240 μg/kg and about 1,200 μg/kg, between about 540 μg/kg and about 1,200 μg/kg, between about 610 μg/kg and about 1,200 μg/kg, between about 650 μg/kg and about 1,200 μg/kg, between about 700 μg/kg and about 1,200 μg/kg, between about 720 μg/kg and about 1,200 μg/kg, between about 750 μg/kg and about 1,200 μg/kg, between about 800 μg/kg and about 1,200 μg/kg, between about 850 μg/kg and about 1,200 μg/kg, between about 900 μg/kg and about 1,200 μg/kg, between about 950 μg/kg and about 1,200 μg/kg, between about 960 μg/kg and between about 1,200 μg/kg, between about 1,000 μg/kg and about 1,200 μg/kg, between about 1,050 μg/kg and about 1,200 μg/kg, between about 1,100 μg/kg and about 1,200 μg/kg, between about 1,200 μg/kg and about 2,000 μg/kg, between about 1,300 μg/kg and about 2,000 μg/kg, between about 1,500 μg/kg and about 2,000 μg/kg, between about 1,700 μg/kg and about 2,000 μg/kg, between about 610 μg/kg and about 1,000 μg/kg, between about 650 μg/kg and about 1,000 μg/kg, between about 700 μg/kg and about 1,000 μg/kg, between about 750 μg/kg and about 1,000 μg/kg, between about 800 μg/kg and about 1,000 μg/kg, between about 850 μg/kg and about 1,000 μg/kg, between about 900 μg/kg and about 1,000 μg/kg, or between about 950 μg/kg and about 1,000 μg/kg.

43. The method of any one of claims 1, 2, and 4 to 42, wherein the IL-7 protein is administered at a dose of between about 60 μg/kg and about 120 μg/kg, between about 120 μg/kg and about 240 μg/kg, between about 240 μg/kg and about 540 μg/kg, between about 540 μg/kg and about 720 μg/kg, between about 720 μg/kg and about 960 μg/kg, between about 700 μg/kg and about 900 μg/kg, between about 750 μg/kg and about 950 μg/kg, between about 700 μg/kg and about 850 μg/kg, between about 750 μg/kg and about 850 μg/kg, between about 700 μg/kg and about 800 μg/kg, between about 800 μg/kg and about 900 μg/kg, between about 750 μg/kg and about 850 μg/kg, or between about 850 μg/kg and about 950 μg/kg.

44. The method of any one of claims 1, 2, and 4 to 43, wherein the IL-7 protein is administered at a dose of about 30 μg/kg, about 60 μg/kg, about 90 μg/kg, about 120 μg/kg, about 150 μg/kg, about 180 μg/kg, about 210 μg/kg, about 240 μg/kg, about 270 μg/kg, about 300 μg/kg, about 330 μg/kg, about 360 μg/kg, about 390 μg/kg, about 420 μg/kg, about 450 μg/kg, about 480 μg/kg, about 510 μg/kg, about 540 μg/kg, about 570 μg/kg, about 600 μg/kg, about 630 μg/kg, about 650 μg/kg, about 680 μg/kg, about 700 μg/kg, about 720 μg/kg, about 740 μg/kg, about 750 μg/kg, about 760 μg/kg, about 780 μg/kg, about 800 μg/kg, about 820 μg/kg, about 840 μg/kg, about 850 μg/kg, about 860 μg/kg, about 880 μg/kg, about 900 μg/kg, about 920 μg/kg, about 940 μg/kg, about 950 μg/kg, about 960 μg/kg, about 980 μg/kg, about 1,000 μg/kg, about 1,020 μg/kg, about 1,040 μg/kg, about 1,060 μg/kg, about 1,080 μg/kg, about 1,100 μg/kg, about 1,120 μg/kg, about 1,140 μg/kg, about 1,160 μg/kg, about 1,180 μg/kg, about 1,200 μg/kg, about 1,220 μg/kg, about 1,240 μg/kg, about 1,260 μg/kg, about 1,280 μg/kg, about 1,300 μg/kg, about 1,320 μg/kg, about 1,340 μg/kg, about 1,360 μg/kg, about 1,380 μg/kg, about 1,400 μg/kg, about 1,420 μg/kg, about 1,440 μg/kg, about 1,460 μg/kg, about 1,480 μg/kg, about 1,500 μg/kg, about 1,520 μg/kg, about 1,540 μg/kg, about 1,560 μg/kg, about 1,580 μg/kg, about 1,600 μg/kg, about 1,620 μg/kg, about 1,640 μg/kg, about 1,660 μg/kg, about 1,680 μg/kg, about 1,700 μg/kg, about 1,720 μg/kg, about 1,740 μg/kg, about 1,760 μg/kg, about 1,780 μg/kg, about 1,800 μg/kg, about 1,820 μg/kg, about 1,840 μg/kg, about 1,860 μg/kg, about 1,880 μg/kg, about 1,900 μg/kg, about 1,920 μg/kg, about 1,940 μg/kg, about 1,960 μg/kg, about 1,980 μg/kg, or about 2,000 μg/kg.

45. The method of any one of claims 1, 2, and 4 to 44, wherein the IL-7 protein is administered at a dose of about 0.01 nmol/kg, about 0.02 nmol/kg, about 0.03 nmol/kg, about 0.04 nmol/kg, about 0.05 nmol/kg, about 0.1 nmol/kg, about 0.2 nmol/kg, about 0.4 nmol/kg, about 0.6 nmol/kg, about 0.8 nmol/kg, about 1 nmol/kg, about 1.2 nmol/kg, about 1.4 nmol/kg, about 1.6 nmol/kg, about 1.8 nmol/kg, about 2 nmol/kg, about 2.2 nmol/kg, about 2.4 nmol/kg, about 2.6 nmol/kg, about 2.8 nmol/kg, about 3 nmol/kg, about 3.5 nmol/kg, about 4 nmol/kg, about 4.5 nmol/kg, about 5 nmol/kg, about 6 nmol/kg, about 7 nmol/kg, about 8 nmol/kg, about 9 nmol/kg, about 10 nmol/kg, about 11 nmol/kg, about 12 nmol/kg, about 13 nmol/kg, about 14 nmol/kg, about 15 nmol/kg, about 16 nmol/kg, about 17 nmol/kg, about 18 nmol/kg, about 19 nmol/kg, about 20 nmol/kg, about 22 nmol/kg, about 24 nmol/kg, about 26 nmol/kg, about 28 nmol/kg, about 30 nmol/kg, about 32 nmol/kg, about 34 nmol/kg, about 36 nmol/kg, about 38 nmol/kg, or about 40 nmol/kg.

46. The method of any one of claims 1, 2, and 4 to 45, wherein the IL-7 protein is administered at a dosing frequency of once about a week, once in about two weeks, once in about three weeks, once in about four weeks, once in about five weeks, once in about six weeks, once in about seven weeks, once in about eight weeks, once in about nine weeks, once in about 10 weeks, once in about 11 weeks, or once in about 12 weeks.

47. The method of claim 46, wherein the IL-7 protein is administered to the subject at a dose of about 720 μg/kg and a dosing frequency of once in about 12 weeks.

48. The method of any one of claims 19 to 47, wherein the additional agent is administered to the subject at a dosing frequency of once about a week, once in about two weeks, once in about three weeks, once in about four weeks, or once in about five weeks.

49. The method of any one of claims 19 to 48, wherein the IL-7 protein and the additional agent are administered to the subject concurrently.

50. The method of any one of claims 19 to 48, wherein the IL-7 protein and the additional agent are administered to the subject sequentially.

51. The method of claim 50, wherein the additional agent is administered to the subject after the IL-7 protein.

52. The method of any one of claims 1, 2, and 4 to 51, wherein the subject receives at least about two doses, at least about three doses, at least about four doses, or at least about five doses of the IL-7 protein.

53. The method of any one of claims 1, 2, and 4 to 52, wherein the subject receives at least about two doses, at least about three doses, at least about four doses, at least about five doses, at least about six doses, or at least about seven doses of the additional agent.

54. The method of claim 53, wherein the subject receives at least about four doses of the IL-7 protein and at least about six doses of the additional agent.

55. The method of any one of claims 52 to 54, wherein the IL-7 protein is administered to the subject once about every 12 weeks and the additional agent is administered to the subject once about every four weeks.

56. The method of any one of claims 53 to 55, wherein one or more doses of the additional agent is administered to the subject over a course of about a single day, over a course of about two consecutive days, over a course of about three consecutive days, over a course of about four consecutive days, over a course of about five consecutive days, over a course of about six consecutive days, or over a course of about seven consecutive days.

57. The method of claim 56, wherein the additional agent is administered to the subject on days 1-5 of a 28-day cycle.

58. The method of any one of claims 1 to 57, wherein the subject has not previously received an anti-tumor therapy.

59. The method of any one of claims 1 to 56, wherein the subject has previously received an anti-tumor therapy.

60. The method of claim 58 or 59, wherein the anti-tumor therapy comprises a standard of care (SOC).

61. The method of claim 59 or 60, wherein the anti-tumor therapy comprises a radiation therapy (RT), chemotherapy, hormone therapy, immunotherapy, photodynamic therapy, stem cell transplant, or combinations thereof.

62. The method of claim 61, wherein the anti-tumor therapy comprises both RT and chemotherapy.

63. The method of claim 62, wherein the chemotherapy comprises temozolomide (TMZ). 64 The method of any one of claims 1, 2, and 4 to 63, wherein the IL-7 protein is administered to the subject intramuscularly, parenthetically, subcutaneously, ophthalmic, intravenously, intraperitoneally, intradermally, intraorbitally, intracerebrally, intracranially, intraspinally, intraventricular, intrathecally, intracistemally, intracapsularly, or intratumorally.

65. The method of any one of claims 19 to 64, wherein the additional agent is administered to the subject intramuscularly, parenthetically, subcutaneously, ophthalmic, intravenously, intraperitoneally, intradermally, intraorbitally, intracerebrally, intracranially, intraspinally, intraventricular, intrathecally, intracistemally, intracapsularly, or intratumorally.
